SEQUENCE LISTING (50 sequences)

SEQ ID NO: 1 - protein, 10 aa, Human papilloma virus
TIHDIILECV

SEQ ID NO: 2 - protein, 10 aa, Human papilloma virus
EVYDFAFRDL

SEQ ID NO: 3 - protein, 10 aa, Human papilloma virus
YMLDLQPETT

SEQ ID NO: 4 - protein, 9 aa, Human papilloma virus
RAHYNIVTF

SEQ ID NO: 5 - protein, 10 aa, Human papilloma virus
LPQLSTELQT

SEQ ID NO: 6 - protein, 8 aa, Human papilloma virus
QLCTELQT

SEQ ID NO: 7 - protein, 7 aa, Human papilloma virus
KQQLLRR

SEQ ID NO: 8 - protein, 10 aa, Human papilloma virus
VYSKQQLLRR

SEQ ID NO: 9 - protein, 10 aa, Human papilloma virus
MHGDTPTLHE

SEQ ID NO: 10 - protein, 6 aa, Human papilloma virus
GQAEPD

SEQ ID NO: 11 - protein, 16 aa, Human papilloma virus
YSKQQLLRREVYDFAF

SEQ ID NO: 12 - protein, 7 aa, Human papilloma virus
YCKQQLL

SEQ ID NO: 13 - protein, 8 aa, Human papilloma virus
CIVYRDGN

SEQ ID NO: 14 - protein, 15 aa, Human papilloma virus
SIVYRDGNPYAVSDK

SEQ ID NO: 15 - protein, 15 aa, Human papilloma virus
DLYCYEQLNDSSEEE

SEQ ID NO: 16 - protein, 20 aa, Human papilloma virus
CCKCDSTLRLCVQSTHVDIR

SEQ ID NO: 17 - protein, 20 aa, Human papilloma virus
SSKSDSTLRLSVQSTHVDIR

SEQ ID NO: 18 - protein, 36 aa, Artificial Sequence
  Synthetic Construct
LPQLSTELQTTIHDIILECVYSKQQLLRREVYDFAF

SEQ ID NO: 19 - protein, 25 aa, Artificial Sequence
  Synthetic Construct
QLCTELQTTIHDIILECVYCKQQLL

SEQ ID NO: 20 - protein, 25 aa, Artificial Sequence
  Synthetic Construct
KQQLLRREVYDFAFRDLCIVYRDGN

SEQ ID NO: 21 - protein, 35 aa, Artificial Sequence
  Synthetic Construct
VYSKQQLLRREVYDFAFRDLSIVYRDGNPYAVSDK

SEQ ID NO: 22 - protein, 35 aa, Artificial Sequence
  Synthetic Construct
MHGDTPTLHEYMLDLQPETTDLYCYEQLNDSSEEE

SEQ ID NO: 23 - protein, 25 aa, Artificial Sequence
  Synthetic Construct
QLCTELQTYMLDLQPETTYCKQQLL

SEQ ID NO: 24 - protein, 35 aa, Artificial Sequence
  Synthetic Construct
GQAEPDRAHYNIVTFCCKCDSTLRLCVQSTHVDIR

SEQ ID NO: 25 - protein, 35 aa, Artificial Sequence
  Synthetic Construct
GQAEPDRAHYNIVTFSSKSDSTLRLSVQSTHVDIR

SEQ ID NO: 26 - DNA, 20 bp, Artificial Sequence
  Synthetic Construct
ggggtcaacgttgagggggg

SEQ ID NO: 27 - DNA, 20 bp, Artificial Sequence
  Synthetic Construct
gggggacgatcgtcgggggg

SEQ ID NO: 28 - DNA, 21 bp, Artificial Sequence
  Synthetic Construct
ggggacgacgtcgtggggggg

SEQ ID NO: 29 - DNA, 20 bp, Artificial Sequence
  Synthetic Construct
tccatgacgttcctgatgct

SEQ ID NO: 30 - DNA, 20 bp, Artificial Sequence
  Synthetic Construct
tccatgacgttcctgacgtt

SEQ ID NO: 31 - DNA, 24 bp, Artificial Sequence
  Synthetic Construct
tcgtcgttttgtcgttttgtcgtt

SEQ ID NO: 32 - DNA, 22 bp, Artificial Sequence
  Synthetic Construct
tcgtcgttgtcgttttgtcgtt

SEQ ID NO: 33 - DNA, 23 bp, Artificial Sequence
  Synthetic Construct
tcgacgttcgtcgttcgtcgttc

SEQ ID NO: 34 - DNA, 26 bp, Artificial Sequence
  Synthetic Construct
tcgcgacgttcgcccgacgttcggta

SEQ ID NO: 35 - DNA, 22 bp, Artificial Sequence
  Synthetic Construct
tcgtcgttttcggcgcgcgccg

SEQ ID NO: 36 - DNA, 25 bp, Artificial Sequence
  Synthetic Construct
tcgtcgtcgttcgaacgacgttgat

SEQ ID NO: 37 - DNA, 29 bp, Artificial Sequence
  Synthetic Construct
tcgcgaacgttcgccgcgttcgaacgcgg

SEQ ID NO: 38 - protein, 35 aa, Artificial Sequence; identical to SEQ ID NO: 22
  Synthetic Construct

SEQ ID NO: 39 - protein, 35 aa, Artificial Sequence
  Synthetic Construct
LYCYEQLNDSSEEEDEIDGPAGQAEPDRAHYNIVT

SEQ ID NO: 40 - protein, 35 aa, Artificial Sequence; identical to SEQ ID NO: 24
  Synthetic Construct

SEQ ID NO: 41 - protein, 35 aa, Artificial Sequence
  Synthetic Construct
TLRLCVQSTHVDIRTLEDLLMGTLGIVCPICSQKP

SEQ ID NO: 42 - protein, 32 aa, Artificial Sequence
  Synthetic Construct
MHQKRTAMFQDPQERPRKLPQLCTELQTTIHD

SEQ ID NO: 43 - protein, 32 aa, Artificial Sequence
  Synthetic Construct
LPQLCTELQTTIHDIILECVYCKQQLLRREVY

SEQ ID NO: 44 - protein, 25 aa, Artificial Sequence
  Synthetic Construct
KQQLLRREVYDFAFRDLCIVYRDGN

SEQ ID NO: 45 - protein, 26 aa, Artificial Sequence
  Synthetic Construct
RDLCIVYRDGNPYAVCDKCLKFYSKI

SEQ ID NO: 46 - protein, 25 aa, Artificial Sequence
  Synthetic Construct
DKCLKFYSKISEYRHYCYSLYGTTL

SEQ ID NO: 47 - protein, 25 aa, Artificial Sequence
  Synthetic Construct
HYCYSLYGTTLEQQYNKPLCDLLIR

SEQ ID NO: 48 - protein, 32 aa, Artificial Sequence
  Synthetic Construct
YGTTLEQQYNKPLCDLLIRCINCQKPLCPEEK

SEQ ID NO: 49 - protein, 32 aa, Artificial Sequence
  Synthetic Construct
RCINCQKPLCPEEKQRHLDKKQRFHNIRGRWT

SEQ ID NO: 50 - protein, 32 aa, Artificial Sequence
  Synthetic Construct
DKKQRFHNIRGRWTGRCMSCCRSSRTRRETQL

(19) United States
(12) Patent Application Publication
(10) Pub. No.: US 2022/0296691 A1
(43) Pub. Date: Sep. 22, 2022

(54) METHODS FOR TREATING CANCER WITH ACTIVATING ANTIGEN CARRIERS

(71) Applicant: SQZ Biotechnologies Company, Watertown, MA (US)

(72) Inventor: Oliver ROSEN, Watertown, MA (US)

(21) Appl. No.: 17/563,787

(22) Filed: Dec. 28, 2021

Related U.S. Application Data
(60) Provisional application No. 63/131,506, filed on Dec. 29, 2020.

(51) Int. Cl. A61K 39/00; A61P 35/00; C07K 14/025

(52) U.S. Cl. CPC A61K 39/001129; A61P 35/00; C07K 14/025; A61K 2039/6087; A61K 2039/6056

(57) ABSTRACT

The present application provides activating antigen carriers (AACs) for treating HPV-associated cancers. AACs are derived from anucleate cells in which at least one antigen and an adjuvant have been delivered intracellularly. In some embodiments, the AAC is administered in combination with a checkpoint inhibitor such as a CTLA4 antagonist and/or a PD-1/PD-L1 agonist.

### CROSS REFERENCE TO RELATED APPLICATIONS

This application claims the benefit of U.S. Provisional Application No. 63/131,506, filed on Dec. 29, 2020, the entire contents of which are incorporated herein by reference.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 750322003500SEQLIST.TXT, date recorded: Dec. 23, 2021, size: 13,033 bytes).

### FIELD OF THE INVENTION

The present disclosure relates generally to methods of using activating antigen carriers (AACs) comprising a HPV antigen and an adjuvant for treating an individual with HPV-associated cancers, doses and regimens thereof. Also disclosed do methods of manufacturing such AACs comprising the at least one HPV antigen and adjuvant, and compositions thereof.

### BACKGROUND OF THE INVENTION

Papillomaviruses are small nonenveloped DNA viruses with a virion size of ˜55 nm in diameter. More than 100 HPV genotypes are completely characterized, and a higher number is presumed to exist. HPV is a known cause of cervical cancers, as well as some vulvar, vaginal, penile, oropharyngeal, anal, and rectal cancers. Although most HPV infections are asymptomatic and clear spontaneously, persistent infections with one of the oncogenic HPV types can progress to precancer or cancer. Other HPV-associated diseases can include common warts, plantar warts, flat warts, anogenital warts, anal lesions, epidermodysplasia, focal epithelial hyperplasia, mouth papillomas, verrucous cysts, laryngeal papillomatosis, squamous intraepithelial lesions (SILs), cervical intraepithelial neoplasia (CIN), vulvar intraepithelial neoplasia (VIN) and vaginal intraepithelial neoplasia (VAIN).

Many of the known human papillomavirus (HPV) types cause benign lesions with a subset being oncogenic. Based on epidemiologic and phylogenetic relationships, HPV types are classified into fifteen “high-risk types” (HPV 16, 18, 31, 33, 35, 39, 45, 51, 52, 56, 58, 59, 68, 73, and 82) and three “probable high-risk types” (HPV 26, 53, and 66), which together are known to manifest as low and high grade cervical changes and cancers, as well as other anogenital cancers such as vulval, vaginal, penile, anal, and perianal cancer, as well as head and neck cancers. Recently, the association of high-risk types HPV 16 and 18 with breast cancer was also described. Eleven HPV types classified as “low risk types” (HPV 6, 11, 40, 42, 43, 44, 54, 61, 70, 72, and 81) are known to manifest as benign low-grade cervical changes, genital warts and recurrent respiratory papillomatosis. Cutaneous HPV types 5, 8, and 92 are associated with skin cancer. In some HPV-associated cancers, the immune system is depressed and correspondingly, the antitumor response is significantly impaired. See Suresh and Burtness Am J Hematol Oncol 13(6):20-27 (2017).

Immunotherapy can be divided generally into two main types of interventions, either passive or active. Passive protocols include administration of pre-activated and/or engineered cells (e.g., CAR T cells), disease-specific therapeutic antibodies, and/or cytokines. Active immunotherapy strategies are directed at stimulating immune system effector functions in vivo. Several current active protocols include vaccination strategies with disease-associated peptides, lysates, or allogeneic whole cells, infusion of autologous dendritic cell (DCs) as vehicles for tumor antigen delivery, and infusion of immune checkpoint modulators. See Papaioannou, Nikos E., et al. Annals of translational medicine 4.14 (2016). Adoptive immunotherapy can be employed to modulate the immune response, enhance antitumor activity, and achieve the goal of treating or preventing HPV-associated cancers.

CD8⁺ cytotoxic T lymphocytes (CTL) and CD4⁺ helper T (Th) cells stimulated by disease-associated antigens have the potential to target and destroy diseased cells; however, current methods for inducing endogenous T cell responses have faced challenges. The methods described herein are used to efficiently generate AACs, which may be anucleate cells or anucleate cell-derived entities comprising HPV antigens and/or adjuvants in a high throughput manner, which can be utilized in inducing a robust T cell response to HPV antigens. The methods described herein also describe methods, treatments, doses and regimens for treating individuals with HPV-associated cancers using AACs comprising HPV antigens and adjuvants.

All references cited herein, including patent applications and publications, are incorporated by reference in their entirety. The patent publications WO 2013/059343, WO 2015/023982, WO 2016/070136, WO2017041050, WO2017008063, WO 2017/192785, WO 2017/192786, WO 2019/178005, WO 2019/178006, WO 2020/072833, WO 2020/154696, and WO 2020/176789, US 20180142198, and US 20180201889 are hereby expressly incorporated by reference in their entirety.

### BRIEF SUMMARY OF THE INVENTION

In some aspects, the invention provides methods for treating a human papilloma virus (HPV)-associated cancer in an individual, the method comprising administering an effective amount of a composition comprising activating antigen carriers (AACs) to the individual wherein the effective amount is about 0.5×10⁸ AACs/kg to about 1×10⁹ AACs/kg, and wherein the AACs comprise at least one HPV antigen and an adjuvant delivered intracellularly. In some aspects, the invention provides methods for treating a human papilloma virus (HPV)-associated cancer in an individual, the method comprising: administering an effective amount of a composition comprising activating antigen carriers (AACs) to the individual, wherein the AACs comprise at least one HPV antigen and an adjuvant delivered intracellularly, and administering an effective amount of an antagonist of CTLA-4 and/or an antagonist of PD-1/PD-L1 to the individual. In some embodiments, the antagonist of CTLA4 is an antibody that binds CTLA4. In some embodiments, the antagonist of PD-1/PD-L1 is an antibody that binds PD-1 or an antibody that binds PD-L1. In some embodiments, an antibody that binds CTLA-4 and an antibody that binds PD-1 are administered to the individual. In some embodiments, the antibody that binds CTLA-4 is ipilimumab. In some embodiments, the antibody that binds PD-1 is nivolumab. In some embodiments, the antibody that binds PD-1 is pembrolizumab. In some embodiments, an antibody that binds CTLA-4 is administered to the individual and an antibody that binds PD-L1 is administered to the individual. In some embodiments, the antibody that binds PD-L1 is atezolizumab.

In some embodiments of the invention, the at least one HPV antigen is a HPV-16 antigen or a HPV-18 antigen. In some embodiments, the at least one HPV antigen comprises a peptide derived from HPV E6 and/or E7. In some embodiments, the at least one HPV antigen comprises an HLA-A2-restricted peptide derived from HPV E6 and/or E7. In some embodiments, the HLA-A2-restricted peptide comprises the amino acid sequence of any one of SEQ ID NOs:1-4. In some embodiments, the at least one HPV antigen comprises the amino acid sequence of any one of SEQ ID NOs:18-25. In some embodiments, the AACs comprise an antigen comprising the amino acid sequence of SEQ ID NO:19 and an antigen comprising the amino acid sequence of SEQ ID NO:23.

In some embodiments, the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist. In some embodiments, the adjuvant is a CpG 7909 oligodeoxynucleotide (ODN).

In some embodiments, the individual is human. In some embodiments, the individual is positive for HLA-A*02. In some embodiments, the AACs are autologous or allogeneic to the individual. In some embodiments, the HPV-associated cancer is a current, locally advanced or metastatic cancer. In some embodiments, the HPV-associated cancer is head and neck cancer, cervical cancer, anal cancer or esophageal cancer. In some embodiments, the composition comprising AACs are administered intravenously. In some embodiments, the antagonist of CTLA-4 and/or antagonist of PD-1/PD-L1 is administered intravenously, orally, or subcutaneously. In some embodiments, the antibody that binds CTLA-4 and/or the antibody that binds PD-1 and/or the antibody that binds PD-L1 is administered intravenously. In some embodiments, the effective amount of AACs comprising the at least one HPV antigen and the adjuvant is about 0.5×10⁸ AACs/kg to about 1×10⁹ AACs/kg. In some embodiments, the effective amount of AACs comprising the at least one HPV antigen and the adjuvant is about 0.5×10⁸ AACs/kg to about 1×10⁹ AACs/kg. In some embodiments, the effective amount of AACs comprising the at least one HPV antigen and the adjuvant is about 0.5×10⁸ AACs/kg, about 2.5×10⁸ AACs/kg, about 5×10⁸ AACs/kg, or about 7.5×10⁸ AACs/kg.

In some embodiments, the effective amount of ipilimumab is about 1 mg/kg to about 3 mg/kg. In some embodiments, the effective amount of nivolumab is about 360 mg. In some embodiments, the effective amount of atezolizumab is about 1200 mg.

In some embodiments, the composition comprising the AACs is delivered on day 1 of a three-week cycle. In some embodiments, the composition comprising the AACs is further administered on day 2 of a first three-week cycle. In some embodiments, about 0.5×10⁸ cells/kg to about 1×10⁹ cells/kg are administered on day 1 of each three-week cycle. In some embodiments, about 0.5×10⁸ cells/kg, about 2.5×10⁸ cells/kg, about 5.0×10⁸ cells/kg, or about 7.5×10⁸ cells/kg are administered on day 1 of each three-week cycle. In some embodiments, about 0.5×10⁸ cells/kg to about 1×10⁹ cells/kg are administered on day 2 of each three-week cycle. In some embodiments, about 0.5×10⁸ cells/kg, about 2.5×10⁸ cells/kg, about 5.0×10⁸ cells/kg, or about 7.5×10⁸ cells/kg are administered on day 2 of the first three-week cycle.

In some embodiments, the antibody that binds CTLA-4 and/or the antibody that binds PD-1 and/or the antibody that binds PD-L1 is administered once per three-week cycle. In some embodiments, the antibody that binds CTLA-4 is administered once per two three-week cycles. In some embodiments, the antibody that binds CTLA-4 is administered on day 1 of each three-week cycle. In some embodiments, the antibody that binds CTLA-4 is ipilimumab, wherein the ipilimumab is administered at a dose of about 3 mg/kg. In some embodiments, the antibody that binds PD-1 is administered on day 8 of the first three-week cycle and day 1 of each subsequent cycle. In some embodiments, the antibody that binds PD-1 is nivolumab, wherein the nivolumab is administered at a dose of about 360 mg. In some embodiments, the antibody that binds CTLA-4 is ipilimumab, wherein the ipilimumab is administered on day 1 of the first three-week cycle of two three-week cycles at a dose of about 1 mg/kg and the antibody that binds PD-1 is administered on day 8 of the first three-week cycle and day 1 of each subsequent cycle at a dose of about 360 mg. In some embodiments, the antibody that binds PD-L1 is administered on day 8 of the first three-week cycle and day 1 of each subsequent cycle. In some embodiments, the antibody that binds PD-L1 is administered at a dose of about 1200 mg. In some embodiments, the composition comprising PBMCs is administered to the individual for at least about three months, six months, nine months or one year.

In some embodiments, the composition comprising AACs comprises about 1×10⁹ AACs to about 1×10¹⁰ AACs in a cryopreservation medium. In some embodiments, the composition comprising AACs comprises about 7×10⁹ PBMCs in about 10 mL of a cryopreservation medium. In some embodiments, the cryopreservation medium is Cryostor® CS2. In some embodiments, the AACs comprising the at least one HPV antigen and an adjuvant are prepared by a process comprising: a) passing a cell suspension comprising a population of input anucleate through a cell-deforming constriction, wherein a diameter of the constriction is a function of a diameter of the input anucleate cells in the suspension, thereby causing perturbations of the input anucleate cells large enough for the at least one HPV antigen and the adjuvant to pass through to form perturbed input anucleate cells; and b) incubating the population of perturbed input anucleate cells with the at least one HPV antigen and the adjuvant for a sufficient time to allow the antigen to enter the perturbed input anucleate cells, thereby generating the AACs comprising the at least one HPV antigen and the adjuvant. In some embodiments, the diameter of the constriction is about 1.6 μm to about 2.4 μm or about 1.8 μm to about 2.2 μm. In some embodiments, the input anucleate cell is a red blood cell. In some embodiments, the at least one HPV antigen comprises a peptide derived from HPV E6 and a peptide derived from HPV E7.

### BRIEF DESCRIPTION OF THE DRAWINGS

FIG. 1 shows the treatment regime for cohort 1.

FIG. 2 shows the treatment regime for cohort 2a.

FIG. 3 shows the treatment regime for cohort 2b.

FIG. 4 shows the treatment regime for cohort 2c.

FIGS. 5A and 5B shows the surface phosphatidylserine levels of AACs comprising an HPV antigen vs. unprocessed red blood cells and as measured by the number of annexin V+ events using FACS. Cells were processed in PBS (FIG. 5A) or RPMI (FIG. 5B).

FIG. 6 shows the percentage of FAM+ events (gated on singlets). AAC-HPV and unprocessed RBCs are used as negative controls. Each point shown per group is derived from data for an individual donor analyzed in a separate experiment denoted by circles. Values shown for the third experiment (right-hand circle for each condition) are averages of data from replicate samples.

FIG. 7 shows the percentage of annexin V+ events (gated on singlets). Unprocessed RBCs are used as negative controls. Each point shown per group is derived from data for an individual donor analyzed in a separate experiment denoted by circles. Values shown for the third experiment (right-hand circle for each condition) are averages of data from replicate samples.

FIG. 8 shows representative images of AAC-HPV (F-E6, E7) from three separate donors are shown (FAM shown in green and PB shown in blue). Graphs displaying normalized (to minimum and maximum signal) FAM (green) and PB (blue) fluorescence intensity along a line drawn across length of AAC-HPV (F-E6, E7) are shown below each image.

FIG. 9 shows representative images of AAC-HPV (F-E6, E7) from three separate donors are shown (FAM shown in green and PB shown in blue). Graphs displaying normalized (to minimum and maximum signal) FAM (green) and PB (blue) fluorescence intensity along a line drawn across length of AAC-HPV (F-E6, E7) are shown below each image.

FIG. 10 shows representative images of AAC-HPV, used as a negative control for FAM fluorescence, from three separate RBC donors are shown (FAM shown in green and PB shown in blue). Graphs displaying normalized FAM (green) and PB (blue) fluorescence intensity along a line drawn across length of AAC-HPV are shown below each image. Display settings in A correspond to display settings used for AAC-HPV (F-E6, E7) in FIG. 8. Display settings in B correspond to display settings used for AAC-HPV (E6, F-E7) in FIG. 9.

FIG. 11 shows PKH26 fluorescence (average±standard deviation of the mean of biological replicates) measured in CD11c+ MODC/PKH26-labeled AAC-HPV co-cultures incubated at 37° C. (red) and 4° C. (blue) and MODC/AAC-HPV co-cultures incubated at 37° C. (green). For display purposes, conditions with no (PKH26-labeled) AAC-HPV were plotted on the x-axis at 0.2. Each graph represents an independent experiment. Each experiment was performed with a distinct human blood and MODC donor.

FIG. 12 presents summary data showing fold change in geometric mean fluorescence intensity (MFI) of CD80, CD83, CD86, and MHC-II surface levels on MODCs 46 hours after co-culture at 37° C. with C-media or AAC-HPV in comparison to control media only. Data from individual MODC donors are shown as different circles. *=P<0.05; **=P<0.01.

FIG. 13 shows graphs depicting IFNγ values secreted from E7-specific CD8⁺ T cells after ˜24 hours of the co-culture with MODCs and media control, SQZ-AAC-HPV, or free E7 SLP. Each graph corresponds to the co-culture with a distinct batch of SQZ-AAC-HPV. MODCs and CD8⁺ T cell co-cultures were incubated with media control, SQZ-AAC-HPV, or free E7 peptide. Each individual data point (full circle) corresponds to an individual well of the co-culture sample. The FP number represents the batch number.

FIG. 14 shows summary data of CD86 geometric MFI of CD11c^{hi}MHC-II^{hi}CD8⁺ cells (CD8⁺ DC), CD11c^{hi}MHC-II^{hi}CD11b⁺ cells (CD11b⁺ DC) and F4/80⁺ CD11b^{lo}/cells (RPM) 14-16 hours after administration of M-AAC-HPV or M-C-media from 2 independent experiments. Each graph represents a separate experiment. Mice that received M-AAC-HPV had higher CD86 geometric MFI in comparison to mice that received M-C-media. *=P<0.05.

FIG. 15 shows summary data of CD83 geometric MFI of CD11c^{hi}MHC-II^{hi}CD8⁺ cells (CD8⁺ DC), CD11c^{hi}MHC-II^{hi}CD11b⁺ cells (CD11b⁺ DC) and F4/80⁺ CD11b^{lo/-} (RPM) 14-16 hours after administration of M-AAC-HPV or M-C-media from 2 independent experiments. CD11c^{hi}MHC-II^{hi}CD8⁺ cells (CD8⁺ DC) and CD11c^{hi}MHC-II^{hi}CD11b⁺ cells (CD11b⁺ DC) had higher CD83 geometric MFI in mice that received M-AAC-HPV compared to mice that received M-C-media. Of note, the CD83 geometric MFI of CD11c^{hi}MHC-II^{hi}CD11b⁺ cells (CD11b⁺ DC) in one experiment had a negative data value and is therefore not displayed on the graph. As noted by Bagwell B S and Park D R, et al, spectral crossover compensation, which is necessary to interpret multiparameter flow cytometry data properly, is a subtractive process. As a result, data values for cell populations that are essentially unstained or are negative for a particular dye, after fluorescence compensation, should be distributed more-or-less normally around a low value. Therefore, data sets resulting from computed compensation commonly (and properly) include populations whose distributions extend below zero. *=P<0.05.

FIG. 16 shows summary data of CD40 geometric MFI of CD11c^{hi}MHC-II^{hi}CD8⁺ cells (CD8⁺ DC), CD11c^{hi}MHC-II^{hi}CD11b⁺ cells (CD11b⁺ DC) and F4/80⁺ CD11b^{lo/-} cells (RPM) 14-16 hours after administration of M-AAC-HPV or M-C-media from 2 independent experiments. Each graph represents a separate experiment. CD11c^{hi}MHC-II^{hi}CD8⁺ cells (CD8⁺ DC) and CD11c^{hi}MHC-II^{hi}CD11b⁺ cells (CD11b⁺ DC) had higher CD40 geometric MFI in mice that received M-AAC-HPV compared to mice that received M-C-media. *=P<0.05.

FIG. 17 shows summary data of CD80 geometric MFI of CD11c^{hi}MHC-II^{hi}CD8⁺ cells (CD8⁺ DC), CD11c^{hi}MHC-II^{hi}CD11b⁺ cells (CD11b⁺ DC) and F4/80⁺ CD11b^{lo/-} cells (RPM) 14-16 hours after administration of M-AAC-HPV or M-C-media from 2 independent experiments. Each graph represents a separate experiment. CD11c^{hi}MHC-II^{hi}CD8⁺ cells (CD8⁺ DC) and CD11c^{hi}MHC-II^{hi}CD11b⁺ cells (CD11b⁺ DC) had higher CD80 geometric MFI in mice that received M-AAC-HPV compared to mice that received M-C-media. *=P<0.05.

FIG. 18 shows summary data of MHC-II geometric MFI of CD11c^{hi}MHC-II^{hi}CD8⁺ cells (CD8⁺ DC), CD11c^{hi}MHC-II^{hi}CD11b⁺ cells (CD11b⁺ DC) and F4/80⁺ CD11b^{lo/-} cells (RPM) 14-16 hours after administration of M-AAC-HPV or M-C-media from 2 independent experiments. Each graph represents a separate experiment. CD11c^{hi}MHC-II^{hi}CD8⁺ cells (CD8+DC) and F4/80⁺ CD11b^{lo/-} cells (RPM) had higher MHC-II geometric MFI in mice that received M-AAC-HPV compared to mice that received M-C-media. NA denotes “not applicable”. *=P<0.05.

FIG. 19 shows the requirement for antigen (E7 SLP) and adjuvant (Poly I:C) in priming E7-specific CD8⁺ T cell responses. The percentage of IFNγ⁺ CD8⁺ T cells is shown. ****=P<0.0001.

FIG. 20 shows the effect of m-AAC-HPV Dose on the magnitude of E7-specific CD8+ T cell responses. Shown is percent of IFNγ⁺ CD44⁺ CD8⁺ T cells. B=10⁹; M=10⁶. n.s.=not statistically significant (P≥0.05); *=P<0.05; ***=P<0.001; ****=P<0.0001.

FIG. 21 shows the percentage of E7-tetramer⁺ activated (CD44^{hi}) CD8⁺ T cells measured in whole blood for each group. Data are presented for the following: PBS (Day 0)-8 days post last dose (Study Day 8); Prime Alone (Day 0)-8 days post last dose (Study Day 8), Boost (Day 2)-8 days post last dose (Study Day 10); Boost (Day 6)-7 days post last dose (Study Day 13). **=P<0.01; ***=P<0.001.

FIG. 22 shows the percentage of E7-tetramer⁺ activated (CD44^{hi}) CD8⁺ T cells measured in whole blood for each group. Data are presented for the following: PBS (Day 0)-13 days post last dose (Study Day 13); Prime Alone (Day 0)-13 days post last dose (Study Day 13), Boost (Day 2)-13 days post last dose (Study Day 15); Boost (Day 6)-14 days post last dose (Study Day 21). **=P<0.01; ****=P<0.0001.

FIG. 23 shows the percentage of E7-tetramer⁺ activated (CD44^{hi}) CD8⁺ T cells measured in whole blood for each group. Data are presented for the following: PBS (Day 0)-21 days post last dose (Study Day 21); Prime Alone (Day 0)-21 days post last dose (Study Day 21), Boost (Day 2)-21 days post last dose (Study Day 23). **=P<0.01.

FIGS. 24A and 24B show summary data of the tumor volume (mean±standard error of the mean) shown for each experimental group over time in two experiments. Lines are terminated at the time at which median survival was reached for the group. ‘M’ denotes million; ‘B’ denotes billion.

FIG. 25 shows survival data for two different experimental groups. Numbers in brackets indicate median survival in days.

FIG. 26 shows summary data of the tumor volume (mean±standard error of the mean) for each experimental group over time. Lines are terminated at the time at which median survival was reached for the group. ‘B’ denotes billion and ‘M’ denotes million. n=10 mice/group.

FIG. 27 shows survival data for experimental groups. Numbers in brackets indicate median survival.

FIG. 28 shows summary data of the tumor volume (mean±standard error of the mean) for each experimental group over time in (A-B) experiment 1 and (C-D) experiment 2. Lines are terminated at the time at which median survival was reached for the group. ‘M’ denotes million. n=10 mice/group.

FIG. 29 shows survival data for two experimental groups. Numbers in brackets indicate median survival in days. ‘M’ denotes million. n=10 mice/group.

FIGS. 30A-30F show the percentage of CD8⁺ T cells is shown as a percentage of the live cell population. (FIG. 30B, FIG. 30E). The percentage of E7-specific (tetramer⁺) cells is shown as a percentage of the live cell population, and (FIG. 30C, FIG. 30F) as a percentage of the CD8⁺ T cell population. Data shown from (FIGS. 30A-30C) Experiment 1 and (FIGS. 30D-30F) Experiment 2. The statistical significance of the M-AAC-HPV-treated group compared to the PBS-treated group is shown (*p<0.05, ***p<0.001, ****p<0.0001).

FIG. 31A-31D show graphs representing the number of CD8⁺ T cells and E7-specific (tetramer⁺) CD8⁺ T cells, respectively, normalized to 100 mg of tumor. The statistical significance of the M-AAC-HPV-treated group compared to the PBS-treated group is shown (**p<0.01, ***p<0.001).

FIG. 32 shows summary data of the mean tumor volume for mice included in each experimental group over time. Immunization with M-AAC-HPV was performed on Day 14 (Experiment 1) or Day 13 (Experiment 2) and indicated as dotted lines. Data (mean±standard error of the mean) is shown up to the time when tumors were harvested (Day 26 for Experiment 2 or Day 25 for Experiment 2). It should be noted that at day 12 post-immunization time point (25 days or 26 days post tumor implant) was selected to ensure enough tumor material would be available for processing and analysis in the treatment group.

FIG. 33 shows the study design for in vivo serum cytokine/chemokine analysis in mice following repeat intravenous administration of m-AAC-HPV as measured by luminex analysis.

FIG. 34 shows the percent of PKH26 labelled cells in whole blood. *=P<0.05 (P value was calculated by comparing percentage of PKH26 labelled cells at the indicated time points in M-AAC-HPV with the corresponding time points in the PBS controls).

### DETAILED DESCRIPTION OF THE INVENTION

In some aspects, the present invention provides methods for treating a human papilloma virus (HPV)-associated cancer in an individual, the method comprising administering an effective amount of a composition comprising activating antigen carriers (AACs) to the individual, wherein the AACs comprise an HPV antigen and an adjuvant delivered intracellularly.

In some aspects, the present invention provides methods for treating a HPV-associated cancer in an individual, the method comprising administering an effective amount of a composition comprising AACs to the individual, wherein the AACs comprise an HPV antigen and an adjuvant delivered intracellularly, and administering an effective amount of one or more immune checkpoint inhibitors. In some embodiments the one or more immune checkpoint inhibitors comprise an antagonist of CTLA-4 (such as but not limited to ipilimumab), an antagonist of PD-1 (such as but not limited to nivolumab), and/or an antagonist of PD-L1 (such as but not limited to atezolizumab).

In some aspects, the present invention provides methods for treating a HPV-associated cancer in an individual, the method comprising administering an effective amount of a composition comprising AACs to the individual, wherein the AACs comprise an HPV antigen and an adjuvant delivered intracellularly, and administering an effective amount of one or more of ipilimumab, nivolumab, or atezolizumab, wherein the AACs comprise the at least one HPV antigen and adjuvant, and/or the one or more immune checkpoint inhibitors are administered in three-week cycles, wherein the effective amount of AACs is about 0.5×10⁸ AACs/kg to about 1×10⁹ AACs/kg, wherein the effective amount of ipilimumab is about 1 mg/kg to about 3 mg/kg, wherein the effective amount of nivolumab is about 360 mg/kg, and wherein the effective amount of atezolizumab is about 1200 mg.

Also provided are compositions of AACs comprising the at least one HPV antigen and adjuvant, and the methods of preparing the AACs comprising the at least one HPV antigen and adjuvant. In some embodiments, the AACs are prepared by a process comprising: a) passing a cell suspension comprising a population of input anucleate through a cell-deforming constriction, wherein a diameter of the constriction is a function of a diameter of the input anucleate cells in the suspension, thereby causing perturbations of the input anucleate cells large enough for the at least one HPV antigen and the adjuvant to pass through to form perturbed input anucleate cells; and b) incubating the population of perturbed input anucleate cells with the at least one HPV antigen and the adjuvant for a sufficient time to allow the antigen and adjuvant to enter the perturbed input anucleate cells, thereby generating the AACs comprising the at least one HPV antigen and the adjuvant. Also provided are compositions for use in inducing an immune response to HPV antigens or for treating a HPV-associated cancer. Also provided are uses of a composition comprising an effective amount of the AACs in the manufacture of a medicament for stimulating an immune response to a HPV antigen or for treating a HPV-associated cancer.

### General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 2011)

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, “anucleate cell” refers to a cell lacking a nucleus. Such cells can include, but are not limited to, platelets, red blood cells (RBCs) such as erythrocytes and reticulocytes. Reticulocytes are immature (e.g., not yet biconcave) red blood cells, typically comprising about 1% of the red blood cells in the human body. Reticulocytes are also anucleate. In certain embodiments, the systems and methods described herein are used the treatment and/or processing of enriched (e.g., comprising a greater percentage of the total cellular population than would be found in nature), purified, or isolated (e.g., from their natural environment, in substantially pure or homogeneous form) populations of anucleate cells (e.g., RBCs, reticulocytes, and/or platelets). In certain embodiments, the systems and methods described herein are used for the treatment and/or processing of whole blood containing RBCs (e.g., erythrocytes or reticulocytes), platelets as well as other blood cells. Purification or enrichment of these cell types is accomplished using known methods such as density gradient systems (e.g., Ficoll-Hypaque), fluorescence activated cell sorting (FACS), magnetic cell sorting, or in vitro differentiation of erythroblasts and erythroid precursors.

The term “vesicle” as used herein refers to a structure comprising liquid enclosed by a lipid bilayer. In some examples, the lipid bilayer is sourced from naturally existing lipid composition. In some examples, the lipid bilayer can be sourced from a cellular membrane. In some examples, vesicles can be derived from various kinds of entities, such as cells. In such examples, a vesicle can retain molecules (such as intracellular proteins or membrane components) from the originating entity. For example, a vesicle derived from a red blood cell may contain any number of intracellular proteins that were in the red blood cell and/or membrane components of the red blood cell. In some examples, a vesicle can contain any number of molecules intracellularly in addition to the desired payload.

As used herein “payload” refers to the material that is being delivered into, such as loaded in, the AAC (e.g., an AAC). “Payload,” “cargo,” “delivery material,” and “compound” are used interchangeably herein. In some embodiments, a payload may refer to a protein, a small molecule, a nucleic acid (e.g., RNA and/or DNA), a lipid, a carbohydrate, a macromolecule, a vitamin, a polymer, fluorescent dyes and fluorophores, carbon nanotubes, quantum dots, nanoparticles, and steroids. In some embodiments, the payload may refer to a protein or small molecule drug. In some embodiments, the payload may comprise one or more compounds.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

The term “heterologous” as it relates to amino acid sequences such as peptide sequences and polypeptide sequences, denotes sequences that are not normally joined together, and/or are not normally associated with a particular cell. Thus, a “heterologous” region of a peptide sequence is a segment of amino acids within or attached to another amino acid molecule that is not found in association with the other molecule in nature. For example, a heterologous region of a peptide construct could include the amino acid sequence of the peptide flanked by sequences not found in association with the amino acid sequence of the peptide in nature. Another example of a heterologous peptide sequence is a construct where the peptide sequence itself is not found in nature (e.g., synthetic sequences having amino acids different as coded from the native gene). Similarly, a cell transformed with a vector that expresses an amino acid construct which is not normally present in the cell would be considered heterologous for purposes of this invention. Allelic variation or naturally occurring mutational events do not give rise to heterologous peptides, as used herein.

The term “exogenous” when used in reference to an agent, such as an antigen or an adjuvant, with relation to a cell or cell-derived vesicle refers to an agent outside of the cell or an agent delivered into the cell from outside the cell. The cell may or may not have the agent already present, and may or may not produce the agent after the exogenous agent has been delivered.

The term “homologous” as used herein refers to a molecule which is derived from the same organism. In some examples the term refers to a nucleic acid or protein which is normally found or expressed within the given organism.

As used herein, “treatment” or “treating” is an approach for obtaining beneficial or desired results, including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the invention contemplate any one or more of these aspects of treatment.

As used herein, the term “prophylactic treatment” refers to treatment, wherein an individual is known or suspected to have or be at risk for having a disorder but has displayed no symptoms or minimal symptoms of the disorder. An individual undergoing prophylactic treatment may be treated prior to onset of symptoms. In some embodiments, an individual may be treated if they have a precancerous lesion, particularly a precancerous lesion associated with HPV infection.

As used herein, by “combination therapy” is meant that a first agent be administered in conjunction with another agent. “In conjunction with” refers to administration of one treatment modality in addition to another treatment modality, such as administration of a composition of nucleated cells as described herein in addition to administration of an immunoconjugate as described herein to the same individual. As such, “in conjunction with” refers to administration of one treatment modality before, during, or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

In the context of cancer, the term “treating” includes any or all of killing cancer cells, inhibiting growth of cancer cells, inhibiting replication of cancer cells, lessening of overall tumor burden and ameliorating one or more symptoms associated with the disease.

As used herein, the term “modulate” may refer to the act of changing, altering, varying, or otherwise modifying the presence, or an activity of, a particular target. For example, modulating an immune response may refer to any act leading to changing, altering, varying, or otherwise modifying an immune response. In some examples, “modulate” refers to enhancing the presence or activity of a particular target. In some examples, “modulate” refers to suppressing the presence or activity of a particular target. In other examples, modulating the expression of a nucleic acid may include, but not limited to a change in the transcription of a nucleic acid, a change in mRNA abundance (e.g., increasing mRNA transcription), a corresponding change in degradation of mRNA, a change in mRNA translation, and so forth.

As used herein, the term “inhibit” may refer to the act of blocking, reducing, eliminating, or otherwise antagonizing the presence, or an activity of, a particular target. Inhibition may refer to partial inhibition or complete inhibition. For example, inhibiting an immune response may refer to any act leading to a blockade, reduction, elimination, or any other antagonism of an immune response. In other examples, inhibition of the expression of a nucleic acid may include, but not limited to reduction in the transcription of a nucleic acid, reduction of mRNA abundance (e.g., silencing mRNA transcription), degradation of mRNA, inhibition of mRNA translation, gene editing and so forth. In other examples, inhibition of the expression of a protein may include, but not be limited to, reduction in the transcription of a nucleic acid encoding the protein, reduction in the stability of mRNA encoding the protein, inhibition of translation of the protein, reduction in stability of the protein, and so forth. In another example, inhibit may refer to the act of slowing or stopping growth; for example, retarding or preventing the growth of a tumor cell.

As used herein, the term “suppress” may refer to the act of decreasing, reducing, prohibiting, limiting, lessening, or otherwise diminishing the presence, or an activity of, a particular target. Suppression may refer to partial suppression or complete suppression. For example, suppressing an immune response may refer to any act leading to decreasing, reducing, prohibiting, limiting, lessening, or otherwise diminishing an immune response. In other examples, suppression of the expression of a nucleic acid may include, but not limited to reduction in the transcription of a nucleic acid, reduction of mRNA abundance (e.g., silencing mRNA transcription), degradation of mRNA, inhibition of mRNA translation, and so forth. In other examples, suppression of the expression of a protein may include, but not be limited to, reduction in the transcription of a nucleic acid encoding the protein, reduction in the stability of mRNA encoding the protein, inhibition of translation of the protein, reduction in stability of the protein, and so forth.

As used herein, the term “enhance” may refer to the act of improving, boosting, heightening, or otherwise increasing the presence, or an activity of, a particular target. For example, enhancing an immune response may refer to any act leading to improving, boosting, heightening, or otherwise increasing an immune response. In one exemplary example, enhancing an immune response may refer to employing an antigen and/or adjuvant to improve, boost, heighten, or otherwise increase an immune response. In other examples, enhancing the expression of a nucleic acid may include, but not limited to increase in the transcription of a nucleic acid, increase in mRNA abundance (e.g., increasing mRNA transcription), decrease in degradation of mRNA, increase in mRNA translation, and so forth. In other examples, enhancing the expression of a protein may include, but not be limited to, increase in the transcription of a nucleic acid encoding the protein, increase in the stability of mRNA encoding the protein, increase in translation of the protein, increase in the stability of the protein, and so forth.

As used herein, the term “induce” may refer to the act of initiating, prompting, stimulating, establishing, or otherwise producing a result. For example, inducing an immune response may refer to any act leading to initiating, prompting, stimulating, establishing, or otherwise producing a desired immune response. In other examples, inducing the expression of a nucleic acid may include, but not limited to initiation of the transcription of a nucleic acid, initiation of mRNA translation, and so forth. In other examples, inducing the expression of a protein may include, but not be limited to, increase in the transcription of a nucleic acid encoding the protein, increase in the stability of mRNA encoding the protein, increase in translation of the protein, increase in the stability of the protein, and so forth.

The term “polynucleotide” or “nucleic acid” as used herein refers to a polymeric form of nucleotides of any length, including ribonucleotides and deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. The backbone of the polynucleotide can comprise repeating units, such as N-(2-aminoethyl)-glycine, linked by peptide bonds (i.e., peptide nucleic acid). Alternatively, the backbone of the polynucleotide can comprise a polymer of synthetic subunits such as phosphoramidates and phorphorthioates and thus can be an oligodeoxynucleoside phosphoramidate (P—NH₂) or a mixed phosphorothioate-phosphorodiester oligomer or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, the term “adjuvant” refers to a substance which modulates and/or engenders an immune response. Generally, the adjuvant is administered in conjunction with an antigen to effect enhancement of an immune response to the antigen as compared to antigen alone. Various adjuvants are described herein.

The terms “CpG oligodeoxynucleotide” and “CpG ODN” herein refer to DNA molecules of 10 to 30 nucleotides in length containing a dinucleotide of cytosine and guanine separated by a phosphate (also referred to herein as a “CpG” dinucleotide, or “CpG”). The CpG ODNs of the present disclosure contain at least one unmethylated CpG dinucleotide. That is, the cytosine in the CpG dinucleotide is not methylated (i.e., is not 5-methylcytosine). CpG ODNs may have a partial or complete phosphorothioate (PS) backbone.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

For any of the structural and functional characteristics described herein, methods of determining these characteristics are known in the art.

As used herein, “microfluidic systems” refers to systems in which low volumes (e.g., mL, nL, pL, fL) of fluids are processed to achieve the discrete treatment of small volumes of liquids. Certain implementations described herein include multiplexing, automation, and high throughput screening. The fluids (e.g., a buffer, a solution, a payload-containing solution, or a cell suspension) can be moved, mixed, separated, or otherwise processed. In certain embodiments described herein, microfluidic systems are used to apply mechanical constriction to a cell suspended in a buffer, inducing perturbations in the cell (e.g., holes) that allow a payload or compound to enter the cytosol of the cell.

As used herein, a “constriction” may refer to a portion of a microfluidic channel defined by an entrance portion, a centerpoint, and an exit portion, wherein the centerpoint is defined by a width, a length, and a depth. In other examples, a constriction may refer to a pore or may be a portion of a pore. The pore may be contained on a surface (e.g., a filter and/or membrane).

For any of the structural and functional characteristics described herein, methods of determining these characteristics are known in the art.

### Methods of Treatment

In some aspects, provided are methods of treating a HPV-associated disease in an individual, the method comprising administering an effective amount of a composition comprising AACs to the individual wherein the AACs comprise an HPV antigen and an adjuvant delivered intracellularly.

In some aspects, provided are methods of treating a HPV-associated disease in an individual, the method comprising administering an effective amount of a composition comprising AACs to the individual wherein the effective amount is about 0.5×10⁷ AAC/kg to about 5×10¹¹ AACs/kg, and wherein the AACs comprise an HPV antigen and an adjuvant delivered intracellularly.

In some embodiments, the HPV-associated disease is an HPV-associated cancer. In some embodiments, the HPV-associated cancer is cervical cancer, perianal cancer, anogenital cancer, oral cancer, salivary cancer, oropharyngeal cancer, vaginal cancer, vulvar cancer, penile cancer, skin cancer or head and neck cancer. In some embodiments, the HPV-associated disease is an HPV-associated infectious disease.

In some embodiments, the effective amount of AACs is about any one of 0.5×10⁶, 1.0×10⁶, 0.5×10⁷, 1.0×10⁷, 0.5×10⁸, 1.0×10⁸, 0.5×10⁹, 1.0×10⁹, 0.5×10¹⁰, 1.0×10¹⁰, 0.5×10¹¹, and 1.0×10¹¹ AACs/kg. In some embodiments, the effective amount is any one of about 0.5×10⁶ to about 1.0×10⁶, about 1.0×10⁶ to about 0.5×10⁷, about 0.5×10⁷ to about 1.0×10⁷, about 1.0×10⁷ to about 0.5×10⁸ AACs, about 0.5×10⁸ to about 1.0×10⁸, about 1.0×10⁸ to about 0.5×10⁹ AACs, about 0.5×10⁹ to about 1.0×10⁹, about 1.0×10⁹ to about 0.5×10¹⁰ AACs, about 0.5×10¹⁰ to about 1.0×10¹⁰, about 1.0×10¹⁰ to about 0.5×10¹¹, or about 0.5×10¹¹ to about 1.0×10¹¹ AACs/kg. In some embodiments, provided are methods of treating a HPV-associated cancer in an individual, the method comprising administering an effective amount of a composition comprising AACs to the individual wherein the effective amount is about 0.5×10⁸ to about 1×10⁹ AACs/kg, and wherein the AACs comprise an HPV antigen and an adjuvant delivered intracellularly.

In some embodiments, the method further comprises administering an effective amount of one or more immune checkpoint inhibitors. Exemplary immune checkpoint inhibitor is an antagonist of, without limitation, PD-1, PD-L1, CTLA-4, LAG3, TIM-3, TIGIT, VISTA, TIM1, B7-H4 (VTCN1) or BTLA. In some embodiments, the immune checkpoint inhibitor is an antagonist of one or more of PD-1, PD-L1, CTLA-4, LAG3, TIM-3, TIGIT, VISTA, TIM1, B7-H4 (VTCN1) or BTLA. In some embodiments, the immune checkpoint inhibitor is one or more of: an antibody that binds to PD-1, an antibody that binds PD-L1, an antibody that binds CTLA-4, an antibody that binds LAG3, an antibody that binds TIM-3, an antibody that binds TIGIT, an antibody that binds VISTA, an antibody that binds TIM-1, an antibody that binds B7-H4, or an antibody that binds BTLA. In further embodiments, the antibody can be a full-length antibody or any variants, for example but not limited to, an antibody fragment, a single chain variable fragment (ScFv), or a fragment antigen binding (Fab). In further embodiments, the antibody can be bispecific, trispecific or multispecific. In some embodiments, the immune checkpoint inhibitor is one or more chemical compounds that binds to and/or inhibits one or more of PD-1, PD-L1, CTLA-4, LAG3, TIM-3, TIGIT, VISTA, TIM1, B7-H4 (VTCN1) or BTLA. In some embodiments, the immune checkpoint inhibitor is one or more peptides that binds to and/or inhibits one or more of PD-1, PD-L1, CTLA-4, LAG3, TIM-3, TIGIT, VISTA, TIM1, B7-H4 (VTCN1) or BTLA. In some embodiments, the immune checkpoint inhibitor is targeted to PD-1. In some embodiments, the immune checkpoint inhibitor is targeted to PD-L1. In some embodiments, the immune checkpoint inhibitor is targeted to CTLA-4.

In some embodiments, provided are methods of treating a HPV-associated cancer in an individual, the method comprising administering an effective amount of a composition comprising AACs to the individual wherein the effective amount is about 0.5×10⁸ to about 1×10⁹ AACs, and wherein the AACs comprise an HPV antigen and an adjuvant delivered intracellularly, and administering an effective amount of one or more immune checkpoint inhibitors. In some embodiments, the immune checkpoint inhibitor is an antagonist of CTLA-4. In some embodiments, the immune checkpoint inhibitor is an antagonist of PD-1. In some embodiments, the immune checkpoint inhibitor is an antagonist of PD-L1. In some embodiments, the one or more immune checkpoint inhibitors comprise an antagonist of CTLA-4, an antagonist of PD-1, and/or an antagonist of PD-L1. In some embodiments, the immune checkpoint inhibitor is an antibody that binds CTLA-4. In some embodiments, the immune checkpoint inhibitor is an antibody that binds PD-1. In some embodiments, the immune checkpoint inhibitor is an antibody that binds PD-L1. In some embodiments, the one or more immune checkpoint inhibitors comprise an antibody that binds CTLA-4, an antibody that binds PD-1, and/or an antibody that binds PD-L1.

In some aspects, provided are methods of treating a HPV-associated disease in an individual, the method comprising administering an effective amount of a composition comprising AACs to the individual wherein the effective amount is about 0.5×10⁸ to about 1×10⁹ AACs, and wherein the AACs comprise an HPV antigen and an adjuvant delivered intracellularly, and administering an effective amount of: an antagonist of CTLA-4, an antagonist of PD-1, and/or an antagonist of PD-L1. In some embodiments, provided are methods of treating a HPV-associated disease in an individual, the method comprising administering an effective amount of a composition comprising AACs to the individual wherein the effective amount is about 0.5×10⁸ to about 1×10⁹ AACs, and wherein the AACs comprise an HPV antigen and an adjuvant delivered intracellularly, and administering an effective amount of: an antibody that binds CTLA-4, an antibody that binds PD-1, and/or an antibody that binds PD-L1. In some embodiments, the antibody that binds PD-1 is nivolumab. In some embodiments, the antibody that binds PD-1 is pembrolizumab. In some embodiments, the antibody that binds PD-L1 is atezolizumab. In some embodiments, the antibody that binds CTLA-4 is ipilimumab. In some embodiments, an antibody that binds CTLA-4 is administered to the individual. In some embodiments, an antibody that binds PD-L1 is administered to the individual. In some embodiments, an antibody that binds PD-1 is administered to the individual.

In some aspects, provided are methods for stimulating an immune response to a HPV antigen in an individual, the method comprising administering an effective amount of a composition comprising AACs (e.g. RBC-derived vesicles) to an individual, wherein the AACs comprise a HPV antigen; wherein the at least one HPV antigen is delivered to the AAC intracellularly. In some embodiments, the AACs further comprise an adjuvant. In some embodiments, the method comprises administering an effective amount of any of the compositions described herein. In some embodiments, the individual has cancer.

In some aspects, provided are methods for reducing tumor growth in an individual, the method comprising administering an effective amount of a composition comprising AACs (e.g. RBC-derived vesicles) to an individual, wherein the AACs comprise a HPV antigen; wherein the at least one HPV antigen is delivered to the AACs intracellularly. In some embodiments, the AACs further comprise an adjuvant. In some embodiments, the method comprises administering an effective amount of any of the compositions described herein. In some embodiments, the individual has cancer.

In some aspects, provided are methods for vaccinating an individual in need thereof, the method comprising administering an effective amount of a composition comprising AACs (e.g. RBC-derived vesicles) to an individual, wherein the AACs comprise a HPV antigen; wherein the at least one HPV antigen is delivered to the AACs intracellularly. In some embodiments, the AACs further comprises an adjuvant. In some embodiments, the method comprises administering an effective amount of any of the compositions described herein. In some embodiments, the individual has cancer.

In some aspects, provided are methods for treating cancer in an individual, the method comprising administering an effective amount of a composition comprising AACs (e.g. RBC-derived vesicles) to an individual, wherein the AACs comprise a HPV antigen; wherein the at least one HPV antigen is delivered to the AACs intracellularly. In some embodiments, the AACs further comprises an adjuvant. In some embodiments, the method comprises administering an effective amount of any of the compositions described herein.

In some aspects, there is provided a method for stimulating an immune response to a HPV antigen in an individual, comprising: a) passing a cell suspension comprising input anucleate cells through a cell-deforming constriction, wherein a diameter of the constriction is a function of a diameter of the input anucleate cells in the suspension, thereby causing perturbations of the input anucleate cells large enough for the at least one HPV antigen and an adjuvant to pass through to form perturbed input anucleate cells; b) incubating the perturbed input anucleate cells with the at least one HPV antigen and the adjuvant for a sufficient time to allow the at least one HPV antigen and the adjuvant to enter the perturbed input anucleate cells; thereby generating AACs comprising the at least one HPV antigen and the adjuvant; and c) administering an effective amount of the AACs comprising the at least one HPV antigen and the adjuvant to the individual.

In some aspects, there is provided a method for reducing tumor growth in an individual, comprising: a) passing a cell suspension comprising input anucleate cells through a cell-deforming constriction, wherein a diameter of the constriction is a function of a diameter of the input anucleate cells in the suspension, thereby causing perturbations of the input anucleate cells large enough for the at least one HPV antigen and an adjuvant to pass through to form perturbed input anucleate cells; b) incubating the perturbed input anucleate cells with the at least one HPV antigen and the adjuvant for a sufficient time to allow the at least one HPV antigen and the adjuvant to enter the perturbed input anucleate cells; thereby generating AACs comprising the at least one HPV antigen and the adjuvant; and c) administering an effective amount of the AACs comprising the at least one HPV antigen and the adjuvant to the individual.

In some aspects, there is provided a method for vaccinating an individual in need thereof, comprising: a) passing a cell suspension comprising input anucleate cells through a cell-deforming constriction, wherein a diameter of the constriction is a function of a diameter of the input anucleate cells in the suspension, thereby causing perturbations of the input anucleate cells large enough for a HPV antigen or the at least one HPV antigen and an adjuvant to pass through to form perturbed input anucleate cells; b) incubating the perturbed input anucleate cells with the at least one HPV antigen and the adjuvant for a sufficient time to allow the at least one HPV antigen and the adjuvant to enter the perturbed input anucleate cells; thereby generating AACs comprising the at least one HPV antigen and an adjuvant; and c) administering an effective amount of the AACs comprising the at least one HPV antigen and the adjuvant to the individual.

In some aspects, there is provided a method for treating cancer in an individual, comprising: a) passing a cell suspension comprising input anucleate cells through a cell-deforming constriction, wherein a diameter of the constriction is a function of a diameter of the input anucleate cells in the suspension, thereby causing perturbations of the input anucleate cells large enough for a HPV antigen and an adjuvant to pass through to form perturbed input anucleate cells; b) incubating the perturbed input anucleate cells with the at least one HPV antigen and the adjuvant for a sufficient time to allow the at least one HPV antigen and the adjuvant to enter the perturbed input anucleate cells; thereby generating AACs comprising the at least one HPV antigen and the adjuvant; and c) administering an effective amount of the AACs comprising the at least one HPV antigen and the adjuvant to the individual.

In some embodiments according to any of the methods, uses or compositions described herein, the methods comprises: a) passing a cell suspension comprising input anucleate cells through a cell-deforming constriction, wherein a diameter of the constriction is a function of a diameter of the input anucleate cells in the suspension, thereby causing perturbations of the input anucleate cells large enough for a HPV antigen to pass through to form perturbed input anucleate cells; b) incubating the perturbed input anucleate cells with the at least one HPV antigen for a sufficient time to allow the at least one HPV antigen to enter the perturbed input anucleate cells; thereby generating AACs comprising the at least one HPV antigen; and c) administering an effective amount of the AACs comprising the at least one HPV antigen to the individual.

In some embodiments, there is provided a composition for stimulating an immune response to HPV protein in an individual, wherein the composition comprises an effective amount of any one of the compositions comprising AACs comprising a HPV antigen as described herein. In some embodiments, there is provided a composition for reducing tumor growth, wherein the composition comprises an effective amount of any one of the compositions comprising AACs comprising a HPV antigen described herein. In some embodiments, the individual has cancer. In some embodiments, there is provided a composition for treating cancer in an individual, wherein the composition comprises an effective amount of any one of the compositions comprising AACs comprising a HPV antigen described herein. In some embodiments, the cancer is cervical cancer, perianal cancer, anogenital cancer, oral cancer, salivary cancer, oropharyngeal cancer, vaginal cancer, vulvar cancer, penile cancer, skin cancer or head and neck cancer.

In some embodiments, there is provided a composition for stimulating an immune response to HPV protein in an individual, wherein the composition comprises an effective amount of any one of the compositions comprising AACs comprising a HPV antigen and an adjuvant as described herein. In some embodiments, there is provided a composition for reducing tumor growth, wherein the composition comprises an effective amount of any one of the compositions comprising AACs comprising a HPV antigen and an adjuvant described herein. In some embodiments, the individual has cancer. In some embodiments, there is provided a composition for treating cancer in an individual, wherein the composition comprises an effective amount of any one of the compositions comprising AACs comprising a HPV antigen and an adjuvant described herein.

In some embodiments, there is provided the use of a composition comprising an effective amount of AACs in the manufacture of a medicament for stimulating an immune response to a HPV antigen, wherein the composition comprises an effective amount of any one of the compositions AACs comprising a HPV antigen described herein. In some embodiments, there is provided the use of a composition comprising an effective amount of AACs in the manufacture of a medicament for reducing tumor growth in an individual, wherein the composition comprises an effective amount of any one of the compositions comprising AACs comprising a HPV antigen described herein. In some embodiments, the individual has cancer. In some embodiments, there is provided the use of a composition comprising an effective amount of AACs in the manufacture of a medicament for treating cancer in an individual, wherein the composition comprises an effective amount any one of the compositions comprising AACs comprising a HPV antigen described herein.

In some embodiments, there is provided the use of a composition comprising an effective amount of AACs in the manufacture of a medicament for stimulating an immune response to HPV antigen protein, wherein the composition comprises an effective amount of any one of the compositions AACs comprising a HPV antigen and an adjuvant described herein. In some embodiments, there is provided the use of a composition comprising an effective amount of AACs in the manufacture of a medicament for reducing tumor growth in an individual, wherein the composition comprises an effective amount of any one of the compositions comprising AACs comprising a HPV antigen and an adjuvant described herein. In some embodiments, the individual has cancer. In some embodiments, there is provided the use of a composition comprising an effective amount of AACs in the manufacture of a medicament for treating cancer in an individual, wherein the composition comprises an effective amount any one of the compositions comprising AACs comprising a HPV antigen and an adjuvant described herein.

In some embodiments according to the methods, uses or compositions described herein, the individual has cancer. In some embodiments, the cancer is cervical cancer, perianal cancer, anogenital cancer, oral cancer, salivary cancer, oropharyngeal cancer, vaginal cancer, vulvar cancer, penile cancer, skin cancer or head and neck cancer. In some embodiments, the cancer is a cancer associated with HPV. In some embodiments, the cancer is a localized cancer. In some embodiments, the cancer is a localized cancer. In some embodiments, the cancer is a locally advanced cancer. In some embodiments, the cancer is a metastatic cancer. In some embodiments, the cancer is a solid tumor. In some embodiments, the cancer is a liquid tumor.

In some embodiments, the width of the constriction is about 10% to about 99% of the mean diameter of the input anucleate cells. In some embodiments, the width of the constriction is any one of about 10% to about 90%, about 10% to about 80%, about 10% to about 70%, about 20% to about 60%, about 40% to about 60%, about 30% to about 45%, about 50% to about 99%, about 50% to about 90%, about 50% to about 80%, about 50% to about 70%, about 60% to about 90%, about 60% to about 80%, or about 60% to about 70% of the mean diameter of the input anucleate cells. In some embodiments, the width of the constriction about 0.25 μm to about 4 μm, about 1 μm to about 4 μm, about 1.2 μm to about 3 μm, about 1.4 μm to about 2.6 μm, about 1.6 μm to about 2.4 μm, or about 1.8 μm to about 2.2. In some embodiments, the width of the constriction is about 2.0 μm. In some embodiments, the width of the constriction is about 2.5 μm. In some embodiments, the width of the constriction is about 3.0 μm. In some embodiments, the width of the constriction is about or less than any one of 0.25 μm, 0.5 μm, 1.0 μm, 1.2 μm, 1.4 μm, 1.6 μm, 1.8 μm, 2.0 μm, 2.2 μm, 2.4 μm, 2.6 μm, 2.8 μm, 3.0 μm, 3.2 μm, 3.4 μm, 3.6 μm, 3.8 μm, 4.0 μm, 4.2 μm, 4.4 μm, 4.6 μm, 4.8 μm, 5.0 μm, 5.2 μm, 5.4 μm, 5.6 μm, 5.8 μm, 6.0 μm. In some embodiments, the cell suspension comprising the input anucleate cells are passed through multiple constrictions wherein the multiple constrictions are arranged in series and/or in parallel.

In some embodiments, the anucleate cell is an RBC or a platelet. In some embodiments, the anucleate cell is an erythrocyte or a reticulocyte. In some embodiments, the AAC is an anucleate cell-derived vesicle. In some embodiments, the AAC is a RBC-derived vesicle or a platelet-derived vesicle. In some embodiments, the AAC is an erythrocyte-derived vesicle or a reticulocyte-derived vesicle.

In some embodiments, the input anucleate cell is autologous to the individual who will receive the composition. In some embodiments, the input anucleate cells is allogeneic to the individual who will receive the composition. In some embodiments, the AAC is autologous to the individual who will receive the composition. In some embodiments, the input AAC is allogeneic to the individual who will receive the composition.

In some embodiments, wherein the AAC comprises an adjuvant, the adjuvant used for conditioning is a CpG oligodeoxynucleotide (ODN), LPS, IFN-α, IFN-β, IFN-γ, alpha-Galactosyl Ceramide, STING agonists, cyclic dinucleotides (CDN), RIG-I agonists, polyinosinic-polycytidylic acid (poly I:C), R837, R848, a TLR3 agonist, a TLR4 agonist or a TLR9 agonist. In some embodiments, the adjuvant is polyinosinic-polycytidylic acid (poly I:C).

In some embodiments, the at least one HPV antigen is a pool of multiple polypeptides that elicit a response against the same and or different HPV antigens. In some embodiments, the at least one HPV antigen is a polypeptide comprising one or more antigenic HPV epitope and one or more heterologous peptide sequences. In some embodiments, the at least one HPV antigen complexes with other antigens or with an adjuvant. In some embodiments, the at least one HPV antigen is capable of being processed into an MHC class I-restricted peptide. In some embodiments, the at least one HPV antigen is capable of being processed into an MHC class II-restricted peptide.

In some embodiments, the method comprises multiple administrations of the AACs comprising the at least one HPV antigen and adjuvant. In some embodiments, the method comprises about 3 to about 9 administrations of the AACs comprising the at least one HPV antigen. In some embodiments, the method comprises about any one of 1, 2, 3, 4, 5, 6, 7, 8, 9, 10, 11, 12, 13, 14 or 15 administrations of the AACs comprising the at least one HPV antigen and adjuvant. In some embodiments, the method comprises continuous administrations of the AACs comprising the at least one HPV antigen and adjuvant as needed. In some embodiments, the time interval between two successive administrations of the AACs comprising the at least one HPV antigen and adjuvant is between about 1 day and about 30 days. In some embodiments, the time interval between two successive administrations of AACs comprising the at least one HPV antigen is about 21 days. In some embodiments, the time the time interval between two successive administrations of the AACs comprising the at least one HPV antigen and adjuvant is about any one of 1, 2, 3, 4, 5, 6, 7, 8, 10, 12, 14, 16, 20, 25, 30, 35, 40, 45, 50, 55, 60, 65, 70, 75, 80, 85, 90, 95, 100, or 150 days. In some embodiments, the time interval between the first two successive administrations of the AACs comprising the at least one HPV antigen and adjuvant is 1 day or 2 days. In some embodiments, the time interval between the first two successive administrations of the AACs comprising the at least one HPV antigen and adjuvant is 1 day or 2 days, wherein the method comprises more than 2 administration of the AACs comprising the at least one HPV antigen and adjuvant (such as but not limited to 3, 4, 5, 6, 7, 8, 9, 10, 11, 12, 13, 14, 15 or more administrations). In some embodiments, the AACs comprising the at least one HPV antigen and adjuvant are administered intravenously, intratumorally, orally and/or subcutaneously. In some embodiments, the AACs comprising the at least one HPV antigen are administered intravenously.

In some embodiments, the composition further comprises an adjuvant. In some embodiments, the adjuvant is a CpG oligodeoxynucleotide (ODN), LPS, IFN-α, IFN-β, IFN-γ, alpha-Galactosyl Ceramide, STING agonists, cyclic dinucleotides (CDN), RIG-I agonists, polyinosinic-polycytidylic acid, R837, R848, a TLR3 agonist, a TLR4 agonist or a TLR9 agonist. In some embodiments, the adjuvant is a CpG oligodeoxynucleotide. In some embodiments, the adjuvant is poly I:C.

In some embodiments, the individual is positive for expression of HLA-A*02, HLA-A*01, HLA-A*03, HLA-A*24, HLA-A*11, HLA-A*26, HLA-A*32, HLA-A*31, HLA-A*68, HLA-A*29, HLA-A*23, HLA-B*07, HLA-B*44, HLA-B*08, HLA-B*35, HLA-B*15, HLA-B*40, HLA-B*27, HLA-B*18, HLA-B*51, HLA-B*14, HLA-B*13, HLA-B*57, HLA-B*38, HLA-C*07, HLA-C*04, HLA-C*03, HLA-C*06, HLA-C*05, HLA-C*12, HLA-C*02, HLA-C*01, HLA-C*08, or HLA-C*16.

Immune checkpoints are regulators of the immune system and keep immune responses in check. Immune checkpoint inhibitors can be employed to facilitate the enhancement of immune response. In some embodiments, the composition comprising the AACs comprising the at least one HPV antigen is administered in combination with administration of an immune checkpoint inhibitor. In some embodiments, the composition comprising the AACs comprising HPV antigen and the immune checkpoint inhibitor are administered simultaneously. In some embodiments, the composition comprising the AACs comprising the at least one HPV antigen and the immune checkpoint inhibitor are administered sequentially. In some embodiments, the immune checkpoint inhibitor and/or the AACs comprising the at least one HPV antigen are administered intravenously, intratumorally, orally and/or subcutaneously. In some embodiments, the AACs comprising the at least one HPV antigen are administered intravenously. In some embodiments, the immune checkpoint inhibitor is administered intravenously, intratumorally, orally and/or subcutaneously.

In some embodiments, the composition comprising the AACs comprising the at least one HPV antigen and adjuvant is administered prior to administration of the immune checkpoint inhibitor. In some embodiments, the composition comprising the AACs comprising the at least one HPV antigen and adjuvant is administered following administration of the immune checkpoint inhibitor. For example, the composition comprising the AACs comprising the at least one HPV antigen and adjuvant is administered from about 1 hour to about 1 week prior to administration of the immune checkpoint inhibitor. For example, in some embodiments, the composition comprising the AACs comprising the at least one HPV antigen and adjuvant is administered about 1 hour, about 2 hours, about 3 hours, about 4 hours, about 6 hours, about 8 hours, about 10 hours, about 12 hours, about 14 hours, about 16 hours, about 18 hours, about 20 hours, about 24 hours, about 30 hours, about 36 hours, about 42 hours, about 48 hours, about 60 hours, about 3 days, about 4 days, about 5 days, about 6 days, or about 7 days prior to administration of the immune checkpoint inhibitor. In some embodiments, the composition comprising the AACs comprising the at least one HPV antigen and adjuvant is administered from between about 1 hour and about 2 hours, from between about 2 hours and about 3 hours, from between about 3 hours and about 4 hours, from between about 4 hours and about 6 hours, from between about 6 hours and about 8 hours, from between about 8 hours and about 10 hours, from between about 10 hours and about 12 hours, from between about 12 hours and about 14 hours, from between about 14 hours and about 16 hours, from between about 16 hours and about 18 hours, from between about 18 hours and about 20 hours, from between about 20 hours and about 24 hours, from between about 24 hours and about 30 hours, from between about 30 hours and about 36 hours, from between about 36 hours and about 42 hours, from between about 42 hours and about 48 hours, from between about 48 hours and about 60 hours, from between about 60 hours and about 3 days, from between about 3 days and about 4 days, from between about 4 days and about 5 days, from between about 5 days and about 6 days, from between about 6 days and about 7 days prior to administration of the immune checkpoint inhibitor.

In some embodiments, the composition comprising the AACs comprising the at least one HPV antigen and adjuvant is administered about 7 days, about 10 days, about 14 days, about 18 days, about 21 days, about 24 days, about 28 days, about 30 days, about 35 days, about 40 days, about 45 days, or about 50 days prior to administration of the immune checkpoint inhibitor. In some embodiments, the composition comprising the AACs comprising the at least one HPV antigen and adjuvant is administered from between about 7 days to about 10 days, from between about 10 days and about 14 days, from between about 14 days and about 18 days, from between about 18 days and about 21 days, from between about 21 days and about 24 days, from between about 24 days and about 28 days, from between about 28 days and about 30 days, from between about 30 days and about 35 days, from between about 35 days and about 40 days, from between about 40 days and about 45 days, or from between about 45 days and about 50 days prior to administration of the immune checkpoint inhibitor.

In some embodiments, the composition comprising the AACs comprising the at least one HPV antigen and adjuvant is administered following administration of the immune checkpoint inhibitor. For example, the composition comprising the AACs comprising the at least one HPV antigen and adjuvant is administered from about 1 hour to about 1 week following administration of the immune checkpoint inhibitor. For example, in some embodiments, the composition comprising the AACs comprising the at least one HPV antigen and adjuvant is administered about 1 hour, about 2 hours, about 3 hours, about 4 hours, about 6 hours, about 8 hours, about 10 hours, about 12 hours, about 14 hours, about 16 hours, about 18 hours, about 20 hours, about 24 hours, about 30 hours, about 36 hours, about 42 hours, about 48 hours, about 60 hours, about 3 days, about 4 days, about 5 days, about 6 days, or about 7 days following administration of the immune checkpoint inhibitor. In some embodiments, the composition comprising the AACs comprising the at least one HPV antigen and adjuvant is administered from between about 1 hour and about 2 hours, from between about 2 hours and about 3 hours, from between about 3 hours and about 4 hours, from between about 4 hours and about 6 hours, from between about 6 hours and about 8 hours, from between about 8 hours and about 10 hours, from between about 10 hours and about 12 hours, from between about 12 hours and about 14 hours, from between about 14 hours and about 16 hours, from between about 16 hours and about 18 hours, from between about 18 hours and about 20 hours, from between about 20 hours and about 24 hours, from between about 24 hours and about 30 hours, from between about 30 hours and about 36 hours, from between about 36 hours and about 42 hours, from between about 42 hours and about 48 hours, from between about 48 hours and about 60 hours, from between about 60 hours and about 3 days, from between about 3 days and about 4 days, from between about 4 days and about 5 days, from between about 5 days and about 6 days, from between about 6 days and about 7 days following administration of the immune checkpoint inhibitor.

In some embodiments, the composition comprising the AACs comprising the at least one HPV antigen and adjuvant is administered about 7 days, about 10 days, about 14 days, about 18 days, about 21 days, about 24 days, about 28 days, about 30 days, about 35 days, about 40 days, about 45 days, or about 50 days following administration of the immune checkpoint inhibitor. In some embodiments, the composition comprising the AACs comprising the at least one HPV antigen and adjuvant is administered from between about 7 days to about 10 days, from between about 10 days and about 14 days, from between about 14 days and about 18 days, from between about 18 days and about 21 days, from between about 21 days and about 24 days, from between about 24 days and about 28 days, from between about 28 days and about 30 days, from between about 30 days and about 35 days, from between about 35 days and about 40 days, from between about 40 days and about 45 days, or from between about 45 days and about 50 days following administration of the immune checkpoint inhibitor.

In some embodiments, the method comprises multiple administration of the composition comprising the AACs comprising the at least one HPV antigen and adjuvant and/or multiple administration of the immune checkpoint inhibitor. For example, in some embodiments, the method comprises two administrations, three administrations, four administrations, five administrations, six administrations, seven administrations, eight administrations, nine administrations, ten administrations, eleven administrations, twelve administrations, thirteen administrations, fourteen administrations, or fifteen administrations of the composition comprising the AACs comprising the at least one HPV antigen and adjuvant and/or the immune checkpoint inhibitor. For example, in some embodiments, the method comprises less than five administrations, less than ten administrations, less than fifteen administrations, less than twenty administrations, less than twenty-five administrations, less than thirty administrations, less than fifty administrations, less than seventy-five administrations, less than one hundred, or less than two hundred administrations of the composition comprising the AACs comprising the at least one HPV antigen and adjuvant and/or the immune checkpoint inhibitor.

Exemplary immune checkpoint inhibitor is targeted to, without limitation, PD-1, PD-L1, CTLA-4, LAG3, TIM-3, TIGIT, VISTA, TIM1, B7-H4 (VTCN1) or BTLA. In some embodiments, the immune checkpoint inhibitor is targeted to one or more of PD-1, PD-L1, CTLA-4, LAG3, TIM-3, TIGIT, VISTA, TIM1, B7-H4 (VTCN1) or BTLA. In some embodiments, the immune checkpoint inhibitor is one or more of: an antibody that binds to PD-1, an antibody that binds PD-L1, an antibody that binds CTLA-4, an antibody that binds LAG3, or an antibody that binds TIM-3, an antibody that binds TIGIT, an antibody that binds VISTA, an antibody that binds TIM-1, an antibody that binds B7-H4, or an antibody that binds BTLA. In further embodiments, the antibody can be a full length antibody or any variants, for example but not limited to, an antibody fragment, a single chain variable fragment (ScFv), or a fragment antigen-binding (Fab). In further embodiments, the antibody can be bispecific, trispecific or multispecific. In some embodiments, the immune checkpoint inhibitor is one or more chemical compounds that binds to and/or inhibits one or more of PD-1, PD-L1, CTLA-4, LAG3, TIM-3, TIGIT, VISTA, TIM1, B7-H4 (VTCN1) or BTLA. In some embodiments, the immune checkpoint inhibitor is one or more peptides that binds to and/or inhibits one or more of PD-1, PD-L1, CTLA-4, LAG3, TIM-3, TIGIT, VISTA, TIM1, B7-H4 (VTCN1) or BTLA. In some embodiments, the immune checkpoint inhibitor is targeted to PD-1. In some embodiments, the immune checkpoint inhibitor is targeted to PD-L1.

In some embodiments, there is provided a plurality of AACs (e.g. RBC-derived vesicles) comprising a HPV antigen and adjuvant for use in a method of stimulating an immune response in an individual according to any one of the methods described herein.

### Compositions of AACs Comprising HPV Antigens

In some embodiments, the AACs comprise an HPV antigen and an adjuvant delivered intracellularly. In some embodiments, the AACs are derived from input anucleate cells. In some embodiments, the AACs are derived from input red blood cells (RBCs). In some embodiments, the AACs are AACs comprising the at least one HPV antigen and the adjuvant. In some embodiments, the AACs are RBC-derived vesicles comprising the at least one HPV antigen and the adjuvant.

In some embodiments, the method comprises administering an effective amount of AACs comprising an HPV antigen and an adjuvant, wherein the AACs comprising the at least one HPV antigen and the adjuvant are prepared by: a) passing a cell suspension comprising input anucleate cells through a cell-deforming constriction, wherein a diameter of the constriction is a function of a diameter of the input anucleate cells in the suspension, thereby causing perturbations of the input anucleate cells large enough for the at least one HPV antigen and the adjuvant to pass through to form perturbed input anucleate cells; and b) incubating the perturbed input anucleate cells with the at least one HPV antigen and the adjuvant for a sufficient time to allow the at least one HPV antigen and the adjuvant to enter the perturbed input anucleate cells; thereby generating AACs comprising the at least one HPV antigen and the adjuvant. In some embodiments, the at least one HPV antigen comprises the amino acid sequence of any one of SEQ ID Nos: 18-25. In some embodiments, the at least one HPV antigen comprises an amino acid sequence with at least 90% identity to any one of SEQ ID Nos: 18-25.

In some aspects, there is provided a composition of AACs comprising a HPV antigen, or a HPV antigen and an adjuvant, wherein the AACs comprising the at least one HPV antigen, or the at least one HPV antigen and the adjuvant are prepared by: a) passing a cell suspension comprising input anucleate cells through a cell-deforming constriction, wherein a diameter of the constriction is a function of a diameter of the input anucleate cells in the suspension, thereby causing perturbations of the input anucleate cells large enough for the at least one HPV antigen and an adjuvant to pass through to form perturbed input anucleate cells; and b) incubating the perturbed input anucleate cells with the at least one HPV antigen and the adjuvant for a sufficient time to allow the at least one HPV antigen to enter the perturbed input anucleate cells; thereby generating AACs comprising the at least one HPV antigen and the adjuvant. In some embodiments, the at least one HPV antigen comprises the amino acid sequence of any one of SEQ ID Nos:18-25. In some embodiments, the at least one HPV antigen comprises an amino acid sequence with at least 90% identity to any one of SEQ ID Nos: 18-25.

In some embodiments, the anucleate cell is an RBC or a platelet. In some embodiments, the anucleate cell is an erythrocyte or a reticulocyte. In some embodiments, the AAC is an anucleate cell-derived vesicle. In some embodiments, the AAC is an RBC-derived vesicle or a platelet-derived vesicle. In some embodiments, the AAC is an erythrocyte-derived vesicle or a reticulocyte-derived vesicle.

In some embodiments, the width of the constriction is about 10% to about 99% of the mean diameter of the input anucleate cells. In some embodiments, the width of the constriction is any one of about 10% to about 90%, about 10% to about 80%, about 10% to about 70%, about 20% to about 60%, about 40% to about 60%, about 30% to about 45%, about 50% to about 99%, about 50% to about 90%, about 50% to about 80%, about 50% to about 70%, about 60% to about 90%, about 60% to about 80%, or about 60% to about 70% of the mean diameter of the input anucleate cells. In some embodiments, the width of the constriction about 0.25 μm to about 4 μm, about 1 μm to about 4 μm, about 1.2 μm to about 3 μm, about 1.4 μm to about 2.6 μm, about 1.6 μm to about 2.4 μm, or about 1.8 μm to about 2.2 μm. In some embodiments, the width of the constriction is about 2.0 μm. In some embodiments, the width of the constriction is about 2.5 μm. In some embodiments, the width of the constriction is about 3.0 μm. In some embodiments, the width of the constriction is about or less than any one of 0.25 μm, 0.5 μm, 1.0 μm, 1.2 μm, 1.4 μm, 1.6 μm, 1.8 μm, 2.0 μm, 2.2 μm, 2.4 μm, 2.6 μm, 2.8 μm, 3.0 μm, 3.2 μm, 3.4 μm, 3.6 μm, 3.8 μm, 4.0 μm, 4.2 μm, 4.4 μm, 4.6 μm, 4.8 μm, 5.0 μm, 5.2 μm, 5.4 μm, 5.6 μm, 5.8 μm, 6.0 μm. In some embodiments, the cell suspension comprising the input anucleate cells are passed through multiple constrictions wherein the multiple constrictions are arranged in series and/or in parallel.

In some embodiments, the at least one HPV antigen is a pool of multiple polypeptides that elicit a response against the same and or different HPV antigens. In some embodiments, the at least one HPV antigen is a polypeptide comprising one or more antigenic HPV epitope and one or more heterologous peptide sequences. In some embodiments, the at least one HPV antigen complexes with other antigens or with an adjuvant. In some embodiments, the at least one HPV antigen is capable of being processed into an MHC class I-restricted peptide. In some embodiments, the at least one HPV antigen is capable of being processed into an MHC class II-restricted peptide.

In some embodiments, the composition further comprises an adjuvant. In some embodiments, the adjuvant is a CpG oligodeoxynucleotide (ODN), LPS, IFN-α, IFN-β, IFN-γ, alpha-Galactosyl Ceramide, STING agonists, cyclic dinucleotides (CDN), RIG-I agonists, polyinosinic-polycytidylic acid (poly I:C), R837, R848, a TLR3 agonist, a TLR4 agonist or a TLR9 agonist. In some embodiments, the adjuvant is polyinosinic-polycytidylic acid (poly I:C).

### Doses and Regimens

In some embodiments, provided are methods of treating a HPV-associated disease in an individual, the method comprising administering an effective amount of a composition comprising AACs to the individual wherein the effective amount is about 0.5×10⁸ to about 1×10⁹ AACs, and wherein the AACs comprise an HPV antigen and an adjuvant delivered intracellularly. In some embodiments, the method further comprises administering an effective amount of one or more immune checkpoint inhibitors.

In some embodiments according to any one of the methods described herein, the effective amount of AACs comprising the at least one HPV antigen and adjuvant is about 0.5×10⁸ AACs/kg to about 1.0×10⁹ AACs/kg. In some embodiments, the effective amount of AACs is about any one of 0.5×10⁶, 1.0×10⁶, 0.5×10⁷, 1.0×10⁷, 0.5×10⁸, 1.0×10⁸, 0.25×10⁹, 0.5×10⁹, 0.75×10⁹, 1.0×10⁹, 0.5×10¹⁰, 1.0×10¹⁰, 0.5×10¹⁰, and 1.0×10¹¹ AACs/kg. In some embodiments, the effective amount of AACs comprising the at least one HPV antigen and the adjuvant is about 0.5×10⁸ AACs/kg, about 2.5×10⁸ AACs/kg, about 5×10⁸ AACs/kg, or about 7.5×10⁸ AACs/kg. In some embodiments, the effective amount of AACs is any one of about 0.5×10⁶ to about 1.0×10⁶, about 1.0×10⁶ to about 0.5×10⁷, about 0.5×10⁷ to about 1.0×10⁷, about 1.0×10⁷ to about 0.5×10⁸ AACs, about 0.5×10⁸ to about 1.0×10⁸, about 1.0×10⁸ to about 0.5×10⁹ AACs, about 0.5×10⁹ to about 1.0×10⁹, about 1.0×10⁹ to about 0.5×10¹⁰ AACs, about 0.5×10¹⁰ to about 1.0×10¹⁰, about 1.0×10¹⁰ to about 0.5×10¹⁰ AACs/kg.

In some embodiments, wherein the method further comprises administering an effective amount of immune checkpoint inhibitor, the immune checkpoint inhibitor is targeted to CTLA-4. In some embodiments, the immune checkpoint inhibitor is ipilimumab. In some embodiments, the effective amount of ipilimumab is about 0.1 mg/kg to about 30 mg/kg. In some embodiments, the effective amount of ipilimumab is any one of about 1 mg/kg to about 3 mg/kg. In some embodiments, the effective amount of ipilimumab is about any one of 0.1, 0.2, 0.5, 1.0, 1.2, 1.4, 1.6, 1.8, 2.0, 2.2, 2.4, 2.6, 2.8, 3, 4, 5, 6, 7, 8, 9, 10, 12, 14, 16, 18, 20, 25, or 30 mg/kg. In some embodiments, the effective amount of ipilimumab is about any one of 0.1 to 0.2, 0.2 to 0.5, 0.5 to 1.0, 1.0 to 1.2, 1.2 to 1.4, 1.4 to 1.6, 1.6 to 1.8, 1.8 to 2.0, 2.0 to 2.2, 2.2 to 2.4, 2.4 to 2.6, 2.6 to 2.8, 2.8 to 3, 3 to 4, 4 to 5, 5 to 6, 6 to 7, 7 to 8, 8 to 9, 9 to 10, 10 to 12, 12 to 14, 14 to 16, 16 to 18, 18 to 20, 20 to 25, or 25 to 30 mg/kg.

In some embodiments, wherein the method further comprises administering an effective amount of immune checkpoint inhibitor, the immune checkpoint inhibitor is targeted to PD-1. In some embodiments, the immune checkpoint inhibitor is nivolumab. In some embodiments, the effective amount of nivolumab is about 30 mg to about 1000 mg. In some embodiments, the effective amount of nivolumab is any one of about 300 mg to about 400 mg. In some embodiments, the effective amount of nivolumab is any one of about 360 mg. In some embodiments, the effective amount of ipilimumab is about any one of 30, 50, 100, 150, 200, 250, 300, 320, 340, 360, 380, 400, 450, 500, 550, 600, 700, 800, 900 or 1000 mg. In some embodiments, the effective amount of ipilimumab is about any one of 30 to 50, 50 to 100, 100 to 150, 150 to 200, 200 to 250, 250 to 300, 300 to 320, 320 to 340, 340 to 360, 360 to 380, 380 to 400, 400 to 450, 500 to 550, 550 to 600, 600 to 700, 700 to 800, 800 to 900, 900 to 1000 mg.

In some embodiments, wherein the method further comprises administering an effective amount of immune checkpoint inhibitor, the immune checkpoint inhibitor is targeted to PD-L1. In some embodiments, the immune checkpoint inhibitor is atezolizumab. In some embodiments, the effective amount of atezolizumab is about 100 mg to about 2500 mg. In some embodiments, the effective amount of atezolizumab is any one of about 900 mg to about 1500 mg. In some embodiments, the effective amount of atezolizumab is any one of about 1200 mg. In some embodiments, the effective amount of atezolizumab is about any one of 100, 200, 300, 400, 500, 600, 700, 800, 900, 1000, 1100, 1150, 1200, 1250, 1300, 1400, 1500, 1600, 1800, 2000, 2200 or 2500 mg. In some embodiments, the effective amount of atezolizumab is about any one of 100 to 200, 200 to 300, 300 to 400, 400 to 500, 500 to 600, 600 to 700, 700 to 800, 800 to 900, 900 to 1000, 1000 to 1100, 1100 to 1200, 1200 to 1300, 1300 to 1400, 1400 to 1500, 1500 to 1600, 1600 to 1800, 1800 to 2000, 2000 to 2200, 2200 to 2500 mg.

In some embodiments, the method of treatment comprises multiple (such as any of 2, 3, 4, 5, 6, 7, 8, 9, 10 or more) cycles of administering any one of the AACs as described herein to the individual. For example, in some embodiments, there is provided a method of vaccinating an individual against an antigen by administering an AAC comprising an antigen and/or an adjuvant, generated by passing an input anucleate cell through a constriction to form an AAC such that the antigen and/or adjuvant enters the AAC, to the individual 2, 3, 4, 5, 6, 7, 8, 9, 10 or more times. In some embodiments, the duration of time between any two consecutive administrations of the AACs is at least about 1 day (such at least about any of 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, or longer, including any ranges between these values).

In some embodiments according to any one of the methods described herein, the composition comprising the AACs is administered in any one of a 1-, 2-, 3-, 4-, 5-, 6-, 7-, 8-, 9-, or 10-week cycle. In some embodiments, the composition comprising the AACs is administered in a 3-week cycle. In some embodiments, the composition comprising the AACs is administered in a 6-week cycle. In some embodiments, the composition comprising the AACs is administered on one or more of day 1, 2, 3, 4, 5, 6, 7, 8, 9, 10, 11, 12, 13, 14, 15, 16, 17, 18, 19, or 20 in the treatment cycle. In some embodiments, the composition comprising the AACs is administered on day 1 of a treatment cycle. In some embodiments, the composition comprising the AACs is administered on day 2 of a treatment cycle. In some embodiments, the composition comprising the AACs is administered on day 1 and day 2 of a treatment cycle. In some embodiments, the composition comprising the AACs is administered on day 1 and day 3 of a treatment cycle. In some embodiments, the composition comprising the AACs is administered on day 8 of a treatment cycle. In some embodiments, the composition comprising the AACs is administered on day 1 of a three-week cycle. In some embodiments, the composition comprising the AACs is further administered on day 2 of a three-week cycle. In some embodiments, the composition comprising the AACs is administered in 3-week cycles until the AAC composition supply is exhausted, or for one year.

In some embodiments, any one of about 0.5×10⁶, 1.0×10⁶, 0.5×10⁷, 1.0×10⁷, 0.5×10⁸, 1.0×10⁸, 0.25×10⁹, 0.5×10⁹, 0.75×10⁹, 1.0×10⁹, 0.5×10¹⁰, 1.0×10¹⁰, 0.5×10¹¹, and 1.0×10¹¹ AACs/kg are administered on day 1 of each three-week cycle. In some embodiments, about 0.5×10⁸ AACs/kg to about 1×10⁹ AACs/kg are administered on day 1 of each three-week cycle. In some embodiments, about 0.5×10⁸ AACs/kg, about 2.5×10⁸ AACs/kg, about 5.0×10⁸ AACs/kg, or about 7.5×10⁸ AACs/kg are administered on day 1 of each three-week cycle. In some embodiments, any one of about 0.5×10⁶, 1.0×10⁶, 0.5×10⁷, 1.0×10⁷, 0.5×10⁸, 1.0×10⁸, 0.25×10⁹, 0.5×10⁹, 0.75×10⁹, 1.0×10⁹, 0.5×10¹⁰, 1.0×10¹⁰, 0.5×10¹¹, and 1.0×10¹¹ AACs/kg are administered on day 2 of each three-week cycle. In some embodiments, about 0.5×10⁸ AACs/kg to about 1×10⁸ AACs/kg are administered on day 2 of each three-week cycle. In some embodiments, about 0.5×10⁸ AACs/kg, about 2.5×10⁸ AACs/kg, about 5.0×10⁸ AACs/kg, or about 7.5×10⁸ AACs/kg are administered on day 2 of each three-week cycle. In some embodiments, 0.5×10⁸ AACs/kg are administered on day 1 of each three-week cycle. In some embodiments, 0.5×10⁸ AACs/kg are administered on day 1 of each three-week cycle, and 0.5×10⁸ AACs/kg are administered on day 2 of each three-week cycle. In some embodiments, 0.5×10⁸ AACs/kg are administered on day 1 of each three-week cycle, and 0.5×10⁸ AACs/kg are administered on day 3 of each three-week cycle. In some embodiments, 2.5×10⁸ AACs/kg are administered on day 1 of each three-week cycle. In some embodiments, 2.5×10⁸ AACs/kg are administered on day 1 of each three-week cycle, and 2.5×10⁸ AACs/kg are administered on day 2 of each three-week cycle. In some embodiments, 2.5×10⁸ AACs/kg are administered on day 1 of each three-week cycle, and 2.5×10⁸ AACs/kg are administered on day 3 of each three-week cycle. In some embodiments, 2.5×10⁸ AACs/kg are administered on day 1 of each three-week cycle. In some embodiments, 5×10⁸ AACs/kg are administered on day 1 of each three-week cycle, and 5×10⁸ AACs/kg are administered on day 2 of each three-week cycle. In some embodiments, 5×10⁸ AACs/kg are administered on day 1 of each three-week cycle, and 5×10⁸ AACs/kg are administered on day 3 of each three-week cycle.

In some embodiments, wherein the method further comprises administering an effective amount of one or more immune checkpoint inhibitors, the immune checkpoint inhibitors are targeted to CTLA-4. PD-1 and/or PD-L1. In some embodiments, the antibody that binds CTLA-4 and/or the antibody that binds PD-1 and/or the antibody that binds PD-L1 is administered 1, 2, 3, 4, 5, 6 or more times per cycle. In some embodiments, the antibody that binds CTLA-4 and/or the antibody that binds PD-1 and/or the antibody that binds PD-L1 is administered once per three-week cycle. In some embodiments, the antibody that binds CTLA-4 is administered once per three week cycle. In some embodiments, the antibody that binds PD-1 is administered once per three week cycle. In some embodiments, the antibody that binds PD-L1 is administered once per three week cycle. In some embodiments, the antibody that binds CTLA-4 and/or the antibody that binds PD-1 and/or the antibody that binds PD-L1 is administered once per two three-week cycles. In some embodiments, the antibody that binds CTLA-4 is administered once per two three week cycles. In some embodiments, the antibody that binds PD-1 is administered once per two three week cycles. In some embodiments, the antibody that binds PD-L1 is administered once per two three week cycles.

In some embodiments, the immune checkpoint inhibitor is administered on one or more times on day 1, 2, 3, 4, 5, 6, 7, 8, 9, 10, 11, 12, 13, 14, 15, 16, 17, 18, 19, or 20 in the treatment cycle. In some embodiments, the immune checkpoint inhibitor is an antibody binding CTLA-4, wherein the antibody that binds CTLA-4 is administered on day 1 of each three-week cycle. In some embodiments, the antibody that binds CTLA-4 is administered for a maximum of four doses. In some embodiments, the effective amount of the antibody that binds CTLA-4 is about 3 mg/kg. In some embodiments, the antibody that binds CTLA-4 is ipilimumab. In some embodiments, the ipilimumab is administered at a dose of about 3 mg/kg. In some embodiments, the antibody that binds CTLA-4 is ipilimumab, wherein the ipilimumab is administered on day 1 of every three-week cycle at a dose of about 3 mg/kg.

In some embodiments, the immune checkpoint inhibitor is an antibody binding PD-1, wherein the antibody that binds PD-1 is administered on day 8 of the first three-week cycle and on day 1 of each subsequent three-week cycle. In some embodiments, the antibody that binds PD-1 is nivolumab. In some embodiments, the nivolumab is administered at a dose of about 360 mg. In some embodiments, the antibody that binds PD-1 is nivolumab, wherein the nivolumab is administered on day 8 of the first three-week cycle and day 1 of each subsequent cycle at a dose of about 360 mg.

In some embodiments, the one of more immune checkpoint inhibitors comprise an antibody binding CTLA-4 and an antibody binding PD-1, wherein the antibody that binds CTLA-4 is administered on day 1 of every alternate three-week cycle (i.e. day 1 of every 6-week cycle) and wherein the antibody that binds PD-1 is administered on day 8 of the first three-week cycle and on day 1 of each subsequent three-week cycle. In some embodiments, the antibody that binds CTLA-4 is ipilimumab and the antibody that binds PD-1 is nivolumab. In some embodiments, the ipilimumab is administered at a dose of about 1 mg/kg. In some embodiments, the nivolumab is administered at a dose of about 360 mg. In some embodiments, the antibody that binds CTLA-4 is ipilimumab, wherein the ipilimumab is administered on day 1 of every alternate three-week cycle at a dose of about 1 mg/kg and the antibody that binds PD-1 is nivolumab, wherein the nivolumab is administered on day 8 of the first three-week cycle and day 1 of each subsequent cycle at a dose of about 360 mg.

In some embodiments, the immune checkpoint inhibitor is an antibody binding PD-L1, wherein the antibody that binds PD-L1 is administered on day 8 of the first three-week cycle and on day 1 of each subsequent three-week cycle. In some embodiments, the antibody that binds PD-L1 is atezolizumab. In some embodiments, the atezolizumab is administered at a dose of about 1200 mg. In some embodiments, the antibody that binds PD-1 is atezolizumab, wherein the atezolizumab is administered on day 8 of the first three-week cycle and day 1 of each subsequent cycle at a dose of about 360 mg.

### Methods of Generating Compositions of AACs Comprising HPV Antigen

In some embodiments, provided are methods for generating a composition comprising AACs comprising a HPV antigen, wherein the at least one HPV antigen is delivered to the AACs intracellularly. In some embodiments, provided are methods for generating a composition comprising AACs comprising a HPV antigen and an adjuvant, wherein the at least one HPV antigen and the adjuvant is delivered to the AACs intracellularly.

In some embodiments, the AACs comprising the at least one HPV antigen and an adjuvant are prepared by a process comprising: a) passing a cell suspension comprising a population of input anucleate through a cell-deforming constriction, wherein a diameter of the constriction is a function of a diameter of the input anucleate cells in the suspension, thereby causing perturbations of the input anucleate cells large enough for the at least one HPV antigen and the adjuvant to pass through to form perturbed input anucleate cells; and b) incubating the population of perturbed input anucleate cells with the at least one HPV antigen and the adjuvant for a sufficient time to allow the antigen to enter the perturbed input anucleate cells, thereby generating the AACs comprising the at least one HPV antigen and the adjuvant.

In some embodiments, the at least one HPV antigen comprises a peptide derived from HPV E6. In some embodiments, the at least one HPV antigen comprises a peptide derived from HPV E7. In some embodiments, the at least one HPV antigen comprises a peptide derived from HPV E6.

In some embodiments, the input anucleate cell is a red blood cell (RBC) or a platelet. In some embodiments, the input anucleate cell is an erythrocyte or a reticulocyte. In some embodiments, the AAC is an anucleate cell-derived vesicle. In some embodiments, the AAC is a RBC-derived vesicle or a platelet-derived vesicle. In some embodiments, the AAC is an erythrocyte-derived vesicle or a reticulocyte-derived vesicle.

In some embodiments, the width of the constriction is about 10% to about 99% of the mean diameter of the input anucleate cells. In some embodiments, the width of the constriction is any one of about 10% to about 90%, about 10% to about 80%, about 10% to about 70%, about 20% to about 60%, about 40% to about 60%, about 30% to about 45%, about 50% to about 99%, about 50% to about 90%, about 50% to about 80%, about 50% to about 70%, about 60% to about 90%, about 60% to about 80%, or about 60% to about 70% of the mean diameter of the input anucleate cells. In some embodiments, the width of the constriction about 0.25 μm to about 4 μm, about 1 μm to about 4 μm, about 1.2 μm to about 3 μm, about 1.4 μm to about 2.6 μm, about 1.6 μm to about 2.4 μm, or about 1.8 μm to about 2.2 μm. In some embodiments, the width of the constriction is about 2.0 μm. In some embodiments, the width of the constriction is about 2.5 μm. In some embodiments, the width of the constriction is about 3.0 μm. In some embodiments, the width of the constriction is about or less than any one of 0.25 μm, 0.5 μm, 1.0 μm, 1.2 μm, 1.4 μm, 1.6 μm, 1.8 μm, 2.0 μm, 2.2 μm, 2.4 μm, 2.6 μm, 2.8 μm, 3.0 μm, 3.2 μm, 3.4 μm, 3.6 μm, 3.8 μm, 4.0 μm, 4.2 μm, 4.4 μm, 4.6 μm, 4.8 μm, 5.0 μm, 5.2 μm, 5.4 μm, 5.6 μm, 5.8 μm, 6.0 μm. In some embodiments, the cell suspension comprising the input anucleate cells are passed through multiple constrictions wherein the multiple constrictions are arranged in series and/or in parallel.

In some embodiments, the at least one HPV antigen is a pool of multiple polypeptides that elicit a response against the same and or different HPV antigens. In some embodiments, the at least one HPV antigen is a polypeptide comprising one or more antigenic HPV epitope and one or more heterologous peptide sequences. In some embodiments, the at least one HPV antigen is delivered with other antigens or with an adjuvant. In some embodiments, the at least one HPV antigen is a polypeptide comprising an antigenic HPV epitope and one or more heterologous peptide sequences. In some embodiments, the at least one HPV antigen complexes with itself, with other antigens, or with the adjuvant. In some embodiments, the at least one HPV is HPV-16 or HPV-18. In some embodiments, the at least one HPV antigen is comprised of an HLA-A2-specific epitope. In some embodiments, the at least one HPV antigen is an HPV E6 antigen or an HPV E7 antigen. In some embodiments, the antigen comprises a peptide derived from HPV E6 and/or E7. In some embodiments, the antigen comprises an HLA-A2-restricted peptide derived from HPV E6 and/or E7. In some embodiments, the at least one HPV antigen is capable of being processed into an MHC class I-restricted peptide. In some embodiments, the at least one HPV antigen is capable of being processed into an MHC class II-restricted peptide.

In some embodiments, the composition further comprises an adjuvant. In some embodiments, the adjuvant is a CpG oligodeoxynucleotide (ODN), LPS, IFN-α, IFN-β, IFN-γ, alpha-Galactosyl Ceramide, STING agonists, cyclic dinucleotides (CDN), RIG-I agonists, polyinosinic-polycytidylic acid (poly I:C), R837, R848, a TLR3 agonist, a TLR4 agonist or a TLR9 agonist. In some embodiments, the adjuvant is polyinosinic-polycytidylic acid (poly I:C).

### HPV Antigens

In some embodiments according to the methods described herein, the exogenous antigen is a human papillomavirus (HPV) antigen. Papillomaviruses are small nonenveloped DNA viruses with a virion size of ˜55 nm in diameter. More than 100 HPV genotypes are completely characterized, and a higher number is presumed to exist. HPV is a known cause of cervical cancers, as well as some vulvar, vaginal, penile, oropharyngeal, anal, and rectal cancers. Although most HPV infections are asymptomatic and clear spontaneously, persistent infections with one of the oncogenic HPV types can progress to precancer or cancer. Other HPV-associated diseases can include common warts, plantar warts, flat warts, anogenital warts, anal lesions, epidermodysplasia, focal epithelial hyperplasia, mouth papillomas, verrucous cysts, laryngeal papillomatosis, squamous intraepithelial lesions (SILs), cervical intraepithelial neoplasia (CIN), vulvar intraepithelial neoplasia (VIN) and vaginal intraepithelial neoplasia (VAIN). Many of the known HPV types cause benign lesions with a subset being oncogenic. Based on epidemiologic and phylogenetic relationships, HPV types are classified into fifteen “high-risk types” (HPV 16, 18, 31, 33, 35, 39, 45, 51, 52, 56, 58, 59, 68, 73, and 82) and three “probable high-risk types” (HPV 26, 53, and 66), which together are known to manifest as low and high grade cervical changes and cancers, as well as other anogenital cancers such as vulval, vaginal, penile, anal, and perianal cancer, as well as head and neck cancers. Recently, the association of high-risk types HPV 16 and 18 with breast cancer was also described. Eleven HPV types classified as “low risk types” (HPV 6, 11, 40, 42, 43, 44, 54, 61, 70, 72, and 81) are known to manifest as benign low-grade cervical changes, genital warts and recurrent respiratory papillomatosis. Cutaneous HPV types 5, 8, and 92 are associated with skin cancer. In some HPV-associated cancers, the immune system is depressed and correspondingly, the antitumor response is significantly impaired. See Suresh and Burtness, Am J Hematol Oncol 13(6):20-27 (2017). In some embodiments, the exogenous antigen is a pool of multiple polypeptides that elicit a response against the same and or different antigens. In some embodiments, an antigen in the pool of multiple antigens does not decrease the immune response directed toward other antigens in the pool of multiple antigens. In some embodiments, the at least one HPV antigen is a polypeptide comprising an antigenic HPV epitope and one or more heterologous peptide sequences. In some embodiments, the at least one HPV antigen complexes with itself, with other antigens, or with the adjuvant. In some embodiments, the at least one HPV is an HPV-16 antigen or a HPV-18 antigen. In some embodiments, the at least one HPV antigen is comprised of an HLA-A2-specific epitope. In some embodiments, the at least one HPV antigen is an HPV E6 antigen or an HPV E7 antigen. In some embodiments, the antigen comprises a peptide derived from HPV E6 and/or E7. In some embodiments, the antigen comprises an HLA-A2-restricted peptide derived from HPV E6 and/or E7. In some embodiments, the antigen comprises an HLA-A2-restricted peptide derived from HPV E6 and/or E7, wherein the HLA-A2 restricted peptide comprises the amino acid sequence of any one of SEQ ID NOs: 1-4. In some embodiments, the HLA-A2 restricted peptide comprises the amino acid sequence of SEQ ID NO: 1. In some embodiments, the HLA-A2 restricted peptide comprises the amino acid sequence of SEQ ID NO: 2. In some embodiments, the HLA-A2 restricted peptide comprises the amino acid sequence of SEQ ID NO: 3. In some embodiments, the HLA-A2 restricted peptide comprises the amino acid sequence of SEQ ID NO: 4. In some embodiments, the HLA-A2-restricted peptide comprises the amino acid sequence of any one of SEQ ID NOs:18-25. In some embodiments, the at least one HPV antigen comprises an amino acid sequence with at least 90% similarity to any one of SEQ ID NOs:18-25. In some embodiments, the at least one HPV antigen comprises an amino acid sequence with at least 90% similarity to SEQ ID NO: 18. In some embodiments, the at least one HPV antigen comprises an amino acid sequence with at least 90% similarity to SEQ ID NO:19. In some embodiments, the at least one HPV antigen comprises the amino acid sequence of SEQ ID NO:20. In some embodiment, the at least one HPV antigen consists of the amino acid sequence of SEQ ID NO:21. In some embodiments, the at least one HPV antigen comprises the amino acid sequence of SEQ ID NO:22. In some embodiments, the at least one HPV antigen consists of the amino acid sequence of SEQ ID NO:23. In some embodiments, the at least one HPV antigen consists of the amino acid sequence of SEQ ID NO:24. In some embodiments, the at least one HPV antigen consists of the amino acid sequence of SEQ ID NO:25. In some embodiments, the at least one HPV antigen comprises the amino acid sequence of any one of SEQ ID NOs:18-25. In some embodiments, the at least one HPV antigen is a plurality of antigens comprising at least one of the amino acid sequences of any one of SEQ ID NOs:18-25. In some embodiments, the exogenous antigen is a plurality of antigens comprising 2, 3, 4, 5, 6, 7 or 8 of the amino acid sequences of any one of SEQ ID Nos:18-25. In some embodiments, the exogenous antigen is a plurality of antigens comprising an amino acid sequence with at least 90% similarity to SEQ ID NO:19 and an amino acid sequence with at least 90% similarity to SEQ ID NO:23 In some embodiments, the exogenous antigen is a plurality of antigens comprising the amino acid sequence of SEQ ID NO:19 and the amino acid sequence of SEQ ID NO:23. In some embodiments, the plurality of antigens is contained within a pool of non-covalently linked peptides. In some embodiments, the plurality of antigens is contained within a pool of non-covalently linked peptides, wherein each peptide comprises no more than one antigen. In some embodiments, the plurality of antigens is contained within a pool of non-covalently linked peptides, wherein the amino acid sequence of SEQ ID NO:19 and the amino acid sequence of SEQ ID NO:25 are contained within separate peptides.

In some embodiments, the at least one HPV antigen is within a pool of multiple polypeptides that elicit a response against the same and or different HPV antigens. In some embodiments, an antigen in the pool of multiple antigens does not decrease the immune response directed toward other antigens in the pool of multiple antigens. In some embodiments, the at least one HPV antigen is a polypeptide comprising an antigenic HPV antigen and one or more heterologous peptide sequences. In some embodiments, the at least one HPV antigen complexes with itself, with other antigens, or with the adjuvant. In some embodiments, the at least one HPV antigen is comprised of an HLA-A2-specific epitope. In some embodiments, the at least one HPV antigen is comprised of an HLA-A11-specific epitope. In some embodiments, HPV antigen is comprised of an HLA-B7-specific epitope. In some embodiments, the at least one HPV antigen is comprised of an HLA-C8-specific epitope. In some embodiments, the at least one HPV antigen comprises part or all of the N-terminal domain of a full-length HPV protein.

In some embodiments according to any one of the methods described herein, the AACs (e.g., RBC-derived vesicles) comprise a plurality of HPV antigens that comprise a plurality of immunogenic epitopes. In further embodiments, following administration to an individual of the AACs comprising the plurality of antigens that comprise the plurality of immunogenic epitopes, none of the plurality of immunogenic epitopes decreases an immune response in the individual to any of the other immunogenic epitopes. In some embodiments, the at least one HPV antigen is a polypeptide and the immunogenic epitope is an immunogenic peptide epitope. In some embodiments, the immunogenic peptide epitope is fused to an N-terminal flanking polypeptide and/or a C-terminal flanking polypeptide. In some embodiments, the at least one HPV antigen is a polypeptide comprising an immunogenic peptide epitope and one or more heterologous peptide sequences. In some embodiments, the at least one HPV antigen is a polypeptide comprising an immunogenic peptide epitope that is flanked on the N-terminus and/or the C-terminus by heterologous peptide sequences. In some embodiments, the flanking heterologous peptide sequences are derived from disease-associated immunogenic peptides. In some embodiments, the flanking heterologous peptide sequences are non-naturally occurring sequence. In some embodiments, the flanking heterologous peptide sequences are derived from an immunogenic synthetic long peptide (SLP). In some embodiments, the at least one HPV antigen is capable of being processed into an MHC class I-restricted peptide and/or an MHC class II-restricted peptide.

### Adjuvants

As used herein, the term “adjuvant” can refer to a substance which either directly or indirectly modulates and/or engenders an immune response. In some embodiments of the invention, an adjuvant is delivered intracellularly to a population of anucleate cells or AACs such as a population of RBCs or RBC-derived vesicles (i.e, incubation of cells or vesicles with an adjuvant before, during and/or after constriction processing, but prior to administration to an individual) to form AACs comprising the adjuvant. In some instances, the adjuvant is administered in conjunction with a HPV antigen to effect enhancement of an immune response to the at least one HPV antigen as compared to HPV antigen alone. Therefore, adjuvants can be used to boost elicitation of an immune cell response (e.g. T cell response) to a HPV antigen. Exemplary adjuvants include, without limitation, stimulator of interferon genes (STING) agonists, retinoic acid-inducible gene I (RIG-I) agonists, and agonists for TLR3, TLR4, TLR7, TLR8 and/or TLR9. Exemplary adjuvants include, without limitation, CpG ODN, interferon-α (IFN-α), polyinosinic:polycytidylic acid (polyL:C), imiquimod (R837), resiquimod (R848), or lipopolysaccharide (LPS). In some embodiments, the adjuvant is CpG ODN, LPS, IFN-α, IFN-β, IFN-γ, alpha-Galactosyl Ceramide, STING agonists, cyclic dinucleotides (CDN), RIG-I agonists, polyinosinic:polycytidylic acid (polyL:C), R837, R848, a TLR3 agonist, a TLR4 agonist or a TLR9 agonist. In particular embodiments, the adjuvant is a CpG ODN. In some embodiments, the adjuvant is a CpG ODN. In some embodiments, the CpG ODN is a Class A CpG ODN, a Class B CpG ODN, or a Class C CpG ODN. In some embodiments, the CpG ODN adjuvant comprise of a selection from the group of CpG ODN 1018, CpG ODN 1585, CpG ODN 2216, CpG ODN 2336, CpG ODN 1668, CpG ODN 1826, CPG ODN 2006, CpG ODN 2007, CpG ODN BW006, CpG ODN D-SL01, CpG ODN 2395, CpG ODN M362, CpG ODN D-SL03. In some embodiments, the CpG ODN adjuvant is CpG ODN 1826 (TCCATGACGTTCCTGACGTT (SEQ ID NO:30)) or CpG ODN 2006 (also known as CpG 7909) (TCGTCGTTTTGTCGTTTTGTCGTT (SEQ ID NO:31)) oligonucleotide. In some embodiments, the adjuvant is CpG 7909. In some embodiments, the RIG-I agonist comprises polyinosinic:polycytidylic acid (polyL:C). Multiple adjuvants can also be used in conjunction with HPV antigens to enhance the elicitation of immune response. In some embodiments, the AACs comprising the at least one HPV antigen further comprise more than one adjuvant. Multiple adjuvants can also be used in conjunction with HPV antigens to enhance the elicitation of immune response. In some embodiments, the AACs comprising the at least one HPV antigen further comprise more than one adjuvant. In some embodiments, the AACs comprising the at least one HPV antigen further comprise any combination of the adjuvants CpG ODN, LPS, IFN-α, IFN-β, IFN-γ, alpha-Galactosyl Ceramide, STING agonists, cyclic dinucleotides (CDN), RIG-I agonists, polyinosinic:polycytidylic acid (polyL:C), R837, R848, a TLR3 agonist, a TLR4 agonist or a TLR9 agonist.

### Further Modifications of AACs Comprising HPV Antigen and Adjuvant

In some embodiments according to any one of the methods described herein, the composition of AACs further comprises an agent that enhances the function of the AACs as compared to a corresponding composition of AACs that does not comprise the agent. In some embodiments, the composition of AACs further comprises an agent that enhances the function of the AACs upon freeze-thaw cycle as compared to a corresponding composition of AACs that does not comprise the agent. In some embodiments, the agent is a cryopreservation agent and/or a hypothermic preservation agent. In some embodiments, the cryopreservation agent nor the hypothermic preservation agent prevents more than 10% or 20% of cell death in a composition of AAC comprising the agent compared to a corresponding composition of AAC that does not comprise the agent before any freeze-thaw cycles. In some embodiments, freeze-thaw cycles of anucleate-cell derived vesicle compositions comprising the cryopreservation agent and/or the hypothermic preservation agent causes not more than 10%, 20%, 30%, 40%, or 50% loss in function when compared to a corresponding composition of anucleate-derived vesicles before the freeze-thaw cycles. In some embodiments, freeze-thaw cycles of anucleate-cell derived vesicle compositions comprising the cryopreservation agent and/or the hypothermic preservation agent causes 10%, 20%, 30%, 40%, or 50% less loss of function when compared to freeze-thaw cycles of a corresponding composition of anucleate-derived vesicles without the cryopreservation agent and the hypothermic preservation agent. In some embodiments, the function or functionality of the anucleate cell-derived vesicle composition is measured by the percentage of the anucleate cell-derived vesicles that are positive for annexin V staining. In some embodiments, the function or functionality of the anucleate cell-derived vesicle composition is measured by the percentage of the anucleate cell-derived vesicles that are positive for CD235a staining. In some embodiments, the function or functionality of the anucleate cell-derived vesicle composition is measured by the percentage of the anucleate cell-derived vesicles that are positive CD235a and annexin V staining. In some embodiments, at least about 70%, about 80%, or about 90% of the AACs are functional after up to 1, 2, 3, 4, 5 freeze-thaw cycles. In some embodiments, the agent is a compound that enhances endocytosis, a stabilizing agent or a co-factor. In some embodiments, the agent is albumin. In some embodiments, the albumin is mouse, bovine, or human albumin. In some embodiments, the agent is one or more of mouse, bovine, or human albumin. In some embodiments, the agent is human albumin. In some embodiments, the agent is one or more of: a divalent metal cation, glucose, ATP, potassium, glycerol, trehalose, D-sucrose, PEG1500, L-arginine, L-glutamine, or EDTA. In some embodiments, the divalent metal cation is one more of Mg2+, Zn2+ or Ca2+. In some embodiments, the agent is one or more of sodium pyruvate, adenine, trehalose, dextrose, mannose, sucrose, human serum albumin (HSA), DMSO, HEPES, glycerol, glutathione, inosine, dibasic sodium phosphate, monobasic sodium phosphate, sodium metal ions, potassium metal ions, magnesium metal ions, chloride, acetate, gluoconate, sucrose, potassium hydroxide, or sodium hydroxide. In some embodiments, the agent is one or more of Sodium pyruvate, adenine, Rejuvesol®, trehalose, dextrose, mannose, sucrose, human serum albumin (HSA), PlasmaLyte®, DMSO, Cryostor® CS2, Cryostor® CS5, Cryostor® CS10, Cryostor® CS15, HEPES, glycerol, glutathione, HypoThermosol®.

In some embodiments according to any one of the methods described herein, the process further comprises a step of incubating the composition of AACs with an agent that enhances the function of the AACs compared to corresponding AACs prepared without the further incubation step.

In some embodiments, the formulation comprises a cryopreservation medium. In some embodiments, the formulation comprises about 1×10⁹ to about 1×10¹¹ AACs in about 9 mL to about 10 mL cryopreservation medium. In some embodiments, the formulation comprises about any one of 0.5×10⁷, 0.7×10⁷, 1.0×10⁷, 0.5×10⁸, 0.7×10⁸, 1.0×10⁸, 0.5×10⁹, 0.7×10⁹, 1.0×10⁹, 0.5×10¹⁰, 0.7×10¹⁰, 1.0×10¹⁰, 0.5×10¹¹, 0.7×10¹¹, 1.0×10¹¹, 0.5×10¹², 0.7×10¹², and 1.0×10¹² AACs in about 9 mL to about 10 mL cryopreservation medium. In some embodiments, the formulation comprises any one of about 0.5×10⁷ to about 1.0×10⁷, about 1.0×10⁷ to about 0.5×10⁸ AACs, about 0.5×10⁸ to about 1.0×10⁸, about 1.0×10⁸ to about 0.5×10⁹ AACs, about 0.5×10⁹ to about 1.0×10⁹, about 1.0×10⁹ to about 0.5×10¹⁰, about 0.5×10¹⁰ to about 1.0×10¹⁰, about 1.0×10¹⁰ to about 0.5×10¹¹, about 0.5×10¹¹ to about 1.0×10¹¹ AACs, about 1.0×10¹¹ to about 0.5×10¹² AACs in about 9 mL to about 10 mL cryopreservation medium. In some embodiments, the formulation comprises about any one of 1×10⁹, 2×10⁹, 3×10⁹, 4×10⁹, 5×10⁹, 6×10⁹, 7×10⁹, 8×10⁹, 9×10⁹, and 1×10¹⁰ AACs in about 9 mL to about 10 mL cryopreservation medium. In some embodiments, the formulation comprises about any one of 1×10⁹, 2×10⁹, 3×10⁹, 4×10⁹, 5×10⁹, 6×10⁹, 7×10⁹, 8×10⁹, 9×10⁹, and 1×10¹⁰ AACs in about 9.5 mL cryopreservation medium. In some embodiments, the formulation comprises about 7×10⁹ AACs in about 9 mL to about 10 mL cryopreservation medium. In some embodiments, the formulation comprises about 7×10⁹ AACs in about 9.5 mL cryopreservation medium. In some embodiments, the formulation comprises about 6.65×10⁹ AACs in about 9.5 mL cryopreservation medium. In some embodiments, the formulation comprising AACs comprise about any one of 0.5×10⁷, 0.7×10⁷, 1.0×10⁷, 0.5×10⁸, 0.7×10⁸, 1.0×10⁸, 0.5×10⁹, 0.7×10⁹, 1.0×10⁹, 0.5×10¹⁰, 0.7×10¹⁰, 1.0×10¹⁰, 0.5×10¹¹, 0.7×10¹¹, 1.0×10¹¹, 0.5×10¹², 0.7×10¹², and 1.0×10¹² AACs in a cryopreservation medium. In some embodiments, the formulation comprises any one of about, about 0.5×10⁷ to about 1.0×10⁷, about 1.0×10⁷ to about 0.5×10⁸ AACs, about 0.5×10⁸ to about 1.0×10⁸, about 1.0×10⁸ to about 0.5×10⁹ AACs, about 0.5×10⁹ to about 1.0×10⁹, about 1.0×10⁹ to about 0.5×10¹⁰ AACs, about 0.5×10¹⁰ to about 1.0×10¹⁰, about 1.0×10¹⁰ to about 0.5×10¹¹, about 0.5×10¹¹ to about 1.0×10¹¹ AACs, about 1.0×10¹¹ to about 0.5×10¹² AACs in a cryopreservation medium. In some embodiments, the formulation comprises about any one of 1×10⁹, 2×10⁹, 3×10⁹, 4×10⁹, 5×10⁹, 6×10⁹, 7×10⁹, 8×10⁹, 9×10⁹, and 1×10¹⁰ AACs in a cryopreservation medium. In some embodiments, the formulation comprises about 7×10⁹ AACs in a cryopreservation medium. In some embodiments, the formulation comprises about 6.65×10⁹ AACs in a cryopreservation medium. In some embodiments, the formulation comprises about 0.7×10⁹ AACs/mL in cryopreservation medium post-thawing. In some embodiments, the formulation comprises about 0.7×10⁹ AACs/mL in cryopreservation medium post-thawing as measured by Coulter counter In some embodiments, the cryopreservation medium comprises CryoStor® CS2. In some embodiments, the cryopreservation medium is CryoStor® CS2.

In some embodiments, the composition comprising AACs comprise about 7×10⁹ AACs in about 9 mL to about 10 mL of CryoStor® CS2. In some embodiments, the composition comprising AACs comprise about 7×10⁹ AACs in about 9.5 mL of CryoStor® CS2. In some embodiments, the formulation comprises about 6.65×10⁹ AACs in about 9.5 mL CryoStor® CS2.

In some embodiments, the AACs in the formulation maintain equal to or greater than about 50% functionality up to 1, 2, 3, 4, 5 freeze-thaw cycles. In some embodiments, the formulation maintain equal to or greater than about 50%, 60%, 70%, 80%, 90%, 95%, or 99% functionality up to 1, 2, 3, 4, 5 freeze-thaw cycles. In some embodiments, the AACs in the formulation maintain equal to or greater than about 70% functionality following storage for at least 12 months at temperatures at or below −140° C. In some embodiments, the formulation maintain equal to or greater than about 50%, 60%, 70%, 80%, 90%, 95%, or 99% functionality following storage for at least 12 months at temperatures at or below −140° C. In some embodiments, the formulation maintain equal to or greater than about 70% functionality following storage for at least 6, 9, 12, 15, 18, 24, 30, or 36 months at temperatures at or below −140° C. In some embodiments, the formulation maintain equal to or greater than about 70% functionality following storage for at least 12 months at temperatures at or below −100° C., −110° C., −120° C., −130° C., −140° C., −150° C., −160° C., −170° C., −180° C., −190° C., or −200° C.

In some embodiments, the AACs in the formulation maintain equal to or greater than about 50% positive staining for annexin V and/or CD235a up to 1, 2, 3, 4, 5 freeze-thaw cycles. In some embodiments, the formulation maintain equal to or greater than about 50%, 60%, 70%, 80%, 90%, 95%, or 99% positive staining for annexin V and/or CD235a up to 1, 2, 3, 4, 5 freeze-thaw cycles. In some embodiments, the AACs in the formulation maintain equal to or greater than about 70% positive staining for annexin V and/or CD235a following storage for at least 12 months at temperatures at or below −140° C. In some embodiments, the formulation maintain equal to or greater than about 50%, 60%, 70%, 80%, 90%, 95%, or 99% positive staining for annexin V and/or CD235a following storage for at least 12 months at temperatures at or below −140° C. In some embodiments, the formulation maintain equal to or greater than about 70% positive staining for annexin V and/or CD235a following storage for at least 6, 9, 12, 15, 18, 24, 30, or 36 months at temperatures at or below −140° C. In some embodiments, the formulation maintain equal to or greater than about 70% positive staining for annexin V and/or CD235a following storage for at least 12 months at temperatures at or below −100° C., −110° C., −120° C., −130° C., −140° C., −150° C., −160° C., −170° C., −180° C., −190° C., or −200° C.

In some embodiments, the AACs in the formulation maintain equal to or greater than about 50% positive staining for annexin V and/or CD235a up to 1, 2, 3, 4, 5 freeze-thaw cycles. In some embodiments, the formulation maintain equal to or greater than about 50%, 60%, 70%, 80%, 90%, 95%, or 99% positive staining for annexin V and/or CD235a up to 1, 2, 3, 4, 5 freeze-thaw cycles. In some embodiments, the AACs in the formulation maintain equal to or greater than about 70% positive staining for annexin V and/or CD235a following storage for at least 12 months at temperatures at or below −140° C. In some embodiments, the formulation maintain equal to or greater than about 50%, 60%, 70%, 80%, 90%, 95%, or 99% positive staining for annexin V and/or CD235a following storage for at least 12 months at temperatures at or below −140° C. In some embodiments, the formulation maintain equal to or greater than about 70% positive staining for annexin V and/or CD235a following storage for at least 6, 9, 12, 15, 18, 24, 30, or 36 months at temperatures at or below −140° C. In some embodiments, the formulation maintain equal to or greater than about 70% positive staining for annexin V and/or CD235a following storage for at least 12 months at temperatures at or below −100° C., −110° C., −120° C., −130° C., −140° C., −150° C., −160° C., −170° C., −180° C., −190° C., or −200° C.

### Constrictions Used in Generating Compositions of AACs Comprising HPV Antigen

In some embodiments, the invention provides compositions of AACs comprising a HPV antigen for stimulating an immune response. In some embodiments, the anucleate cell is an RBC or a platelet. In some embodiments, the anucleate cell is an erythrocyte or a reticulocyte. In some embodiments, the at least one HPV antigen is delivered to the anucleate cells intracellularly. Methods of introducing payloads to anucleate cells are known in the art.

In some embodiments, the at least one HPV antigen is introduced into the anucleate cells by passing the cell through a constriction such that transient pores are introduced to the membrane of the cell thereby allowing the at least one HPV antigen to enter the cell. Examples of constriction-based delivery of compounds into a cell are provided by WO 2013/059343, WO 2015/023982, WO 2016/070136, WO2017041050, WO2017008063, WO 2017/192785, WO 2017/192786, WO 2019/178005, WO 2019/178006, WO 2020/072833, WO 2020/154696, and WO 2020/176789, US 20180142198, and US 20180201889.

In some embodiments, the at least one HPV antigen and adjuvant are delivered into the anucleate cells to produce the AACs of the invention by passing a cell suspension comprising the anucleate cells (e.g., RBCs) through a constriction, wherein the constriction deforms the cells thereby causing a perturbation of the cells such that a HPV antigen and an adjuvant enter the cells. In some embodiments, the constriction is contained within a microfluidic channel. In some embodiments, multiple constrictions can be placed in parallel and/or in series within the microfluidic channel.

In some embodiments, the constriction within the microfluidic channel includes an entrance portion, a center point, and an exit portion. In some embodiments, the length, depth, and width of the constriction within the microfluidic channel can vary. In some embodiments, the width of the constriction within the microfluidic channel is a function of the diameter of the anucleate cells. Methods to determine the diameter of anucleate cells are known in the art; for example, high-content imaging, cell counters or flow cytometry.

In some embodiments of the constriction-based delivery of an HPV antigen to AACs, the width of the constriction is about 0.5 μm to about 10 μm. In some embodiments, the width of the constriction is about 1 μm to about 4 μm. In some embodiments, the width of the constriction is about 1 μm to about 3 μm. In some embodiments, the width of the constriction is about 1.5 μm to about 2.5 μm. In some embodiments, the width of the constriction is about 1.2 μm to about 2.8 μm. In some embodiments, the width of the constriction is about 0.5 μm to about 5 μm. In some embodiments, the width of the constriction is about 2 μm to about 2.5 μm. In some embodiments, the width of the constriction is about 1.5 μm to about 2 μm. In some embodiments, the width of the constriction is about 0.5 μm to about 3.5 μm. In some embodiments, the width of the constriction is about 3.2 μm to about 3.8 μm. In some embodiments, the width of the constriction is about 3.8 μm to about 4.3 μm. In some embodiments, the width of the constriction is about or less than any one of 0.25 μm, 0.5 μm, 1.0 μm, 1.2 μm, 1.4 μm, 1.6 μm, 1.8 μm, 2.0 μm, 2.2 μm, 2.4 μm, 2.6 μm, 2.8 μm, 3.0 μm, 3.2 μm, 3.4 μm, 3.6 μm, 3.8 μm, 4.0 μm, 4.2 μm, 4.4 μm, 4.6 μm, 4.8 μm, 5.0 μm, 5.2 μm, 5.4 μm, 5.6 μm, 5.8 μm, 6.0 μm. In some embodiments, the width of the constriction is about 2 μm. In some embodiments, the width of the constriction is about 2.2 μm. In some embodiments, the width of the constriction is about 2.5 μm. In some embodiments, the width of the constriction is about 3 μm.

Examples of parameters that may influence the delivery of the compound into the AACs include, but are not limited to, the dimensions of the constriction, the entrance angle of the constriction, the surface properties of the constrictions (e.g., roughness, chemical modification, hydrophilic, hydrophobic, etc.), the operating flow speeds (e.g., cell transit time through the constriction), the cell concentration, the concentration of the compound in the cell suspension, buffer in the cell suspension, and the amount of time that the AACs recover or incubate after passing through the constrictions can affect the passage of the delivered compound into the AACs. Additional parameters influencing the delivery of the compound into the AACs can include the velocity of the input anucleate cells in the constriction, the shear rate in the constriction, the viscosity of the cell suspension, the velocity component that is perpendicular to flow velocity, and time in the constriction. In addition, multiple chips comprising channels in series and/or in parallel may impact delivery to AACs. Multiple chips in parallel may be useful to enhance throughput. Such parameters can be designed to control delivery of the compound. In some embodiments, the cell concentration ranges from about 10 to at least about 10¹² cells/mL or any concentration or range of concentrations therebetween. In some embodiments, delivery compound concentrations can range from about 10 ng/mL to about 1 g/mL or any concentration or range of concentrations therebetween. In some embodiments, delivery compound concentrations can range from about 1 pM to at least about 2 M or any concentration or range of concentrations therebetween.

In some embodiments, the concentration of HPV antigen incubated with the anucleate cells or anucleate-cell derived vesicles is between about 0.01 μM and about 10 mM. For example, in some embodiments, the concentration of HPV antigen incubated with the anucleate cells or AACs is any of less than about 0.01 μM, about 0.1 μM, about 1 μM, about 10 μM, about 100 μM, about 1 mM or about 10 mM. In some embodiments, the concentration of HPV antigen incubated with the anucleate cells or AACs is greater than about 10 mM. In some embodiments, the concentration of HPV antigen incubated with the anucleate cells or AACs is any of between about 0.01 μM and about 0.1 μM, between about 0.1 μM and about 1 μM, between about 1 μM and about 10 μM, between about 10 μM and about 100 μM, between about 100 μM and about 1 mM, or between 1 mM and about 10 mM. In some embodiments, the concentration of HPV antigen incubated with the anucleate cells or AACs is between about 0.1 μM and about 1 mM. In some embodiments, the concentration of HPV antigen incubated with the anucleate cells or AACs is between about 0.1 μM and about 10 μM. In some embodiments, the concentration of HPV antigen incubated with the anucleate cells or AACs is 1 μM.

In some embodiments, the concentration of antigen incubated with the perturbed input anucleate cell is between about 0.01 μM and about 10 mM. For example, in some embodiments, the concentration of antigen incubated with the perturbed input anucleate cell is any of less than about 0.01 μM, about 0.1 μM, about 1 μM, about 10 μM, about 100 μM, about 1 mM or about 10 mM. In some embodiments, the concentration of antigen incubated with the perturbed input anucleate cell is greater than about 10 mM. In some embodiments, the concentration of antigen incubated with the perturbed input anucleate cell is any of between about 0.01 μM and about 0.1 μM, between about 0.1 μM and about 1 μM, between about 1 μM and about 10 μM, between about 10 μM and about 100 μM, between about 100 μM and about 1 mM, or between 1 mM and about 10 mM. In some embodiments, the concentration of antigen incubated with the perturbed input anucleate cell is between about 0.1 μM and about 1 mM. In some embodiments, the concentration of antigen incubated with the perturbed input Anucleate cell is between about 0.1 μM and about 10 μM. In some embodiments, the concentration of antigen incubated with the perturbed input anucleate cell is 1 μM.

In some embodiments, the molar ratio of antigen to adjuvant incubated with the perturbed input anucleate cell is any of between about 10000:1 to about 1:10000. For example, in some embodiments, the molar ratio of antigen to adjuvant incubated with the perturbed input anucleate cell is about any of 10000:1, about 1000:1, about 100:1, about 10:1, about 1:1, about 1:10, about 1:100, about 1:1000, or about 1:10000. In some embodiments, the molar ratio of antigen to adjuvant incubated with the perturbed input anucleate cell is any of between about 10000:1 and about 1000:1, between about 1000:1 and about 100:1, between about 100:1 and about 10:1, between about 10:1 and about 1:1, between about 1:1 and about 1:10, between about 1:10 and about 1:100, between about 1:100 and about 1:1000, between about 1:1000 and about 1:10000. In some embodiments, the molar ratio of antigen to adjuvant incubated with the perturbed input anucleate cell is about 200:1. In some embodiments, the molar ratio of antigen to adjuvant incubated with the perturbed input anucleate cell is about 20:1.

In some embodiments, the AACs comprise the adjuvant at a concentration between about 1 nM and about 1 mM. For example, in some embodiments, the AACs comprise the adjuvant at a concentration of any of less than about 0.01 μM, about 0.1 μM, about 1 μM, about 10 μM, about 100 μM, about 1 mM or about 10 mM. In some embodiments, the AACs comprise the adjuvant at a concentration of greater than about any of 10 mM. In some embodiments, the AACs comprise the adjuvant at a concentration of any of between about 1 nM to about 10 nM, about 0.1 μM and about 1 μM, between about 1 μM and about 10 μM, between about 10 μM and about 100 μM, between about 100 μM and about 1 mM, or between 1 mM and about 10 mM. In some embodiments, the AACs comprise the adjuvant at a concentration between about 0.1 μM and about 1 mM. In some embodiments, the AACs comprise the adjuvant at a concentration of about 1 μM.

In some embodiments, the AACs comprise the antigen at a concentration between about 1 nM and about 1 mM. For example, in some embodiments, the AACs comprises the antigen at a concentration of any of less than about 0.01 μM, about 0.1 μM, about 1 μM, about 10 μM, about 100 μM, about 1 mM or about 10 mM. In some embodiments, the AACs comprise the antigen at a concentration of greater than about any of 10 mM. In some embodiments, the AACs comprise the antigen at a concentration of any of between about 1 nM to about 10 nM, about 0.1 μM and about 1 μM, between about 1 μM and about 10 μM, between about 10 μM and about 100 μM, between about 100 μM and about 1 mM, or between 1 mM and about 10 mM. In some embodiments, the AACs comprise the antigen at a concentration between about 0.1 μM and about 1 mM. In some embodiments, the AACs comprise the antigen at a concentration of about 1 μM.

In some embodiments, the molar ratio of antigen to adjuvant in the AACs is any of between about 10000:1 to about 1:10000. For example, in some embodiments, the molar ratio of antigen to adjuvant in the AACs is about any of 10000:1, about 1000:1, about 100:1, about 10:1, about 1:1, about 1:10, about 1:100, about 1:1000, or about 1:10000. In some embodiments, the molar ratio of antigen to adjuvant in the modified PBMCs is any of between about 10000:1 and about 1000:1, between about 1000:1 and about 100:1, between about 100:1 and about 10:1, between about 10:1 and about 1:1, between about 1:1 and about 1:10, between about 1:10 and about 1:100, between about 1:100 and about 1:1000, between about 1:1000 and about 1:10000. In some embodiments, the molar ratio of antigen to adjuvant in the AACs is about 200:1. In some embodiments, the molar ratio of antigen to adjuvant in the AACs is about 20:1.

### Characteristics of AACs and Internalization by Antigen Presenting Cells

In embodiments according to any one of the methods, uses or compositions described herein, the method comprises: a) passing a cell suspension comprising input anucleate cells through a cell-deforming constriction, wherein a diameter of the constriction is a function of a diameter of the input anucleate cells in the suspension, thereby causing perturbations of the input anucleate cells large enough for an HPV antigen and an adjuvant to pass through to form perturbed input anucleate cells; b) incubating the perturbed input anucleate cells with the at least one HPV antigen and adjuvant for a sufficient time to allow the at least one HPV antigen and adjuvant to enter the perturbed input anucleate cells; thereby generating AACs comprising the at least one HPV antigen and adjuvant. In some embodiments, the AACs comprising the payload (such as HPV antigen and adjuvant) displays different characteristics compared to an input anucleate cell. In some embodiments, the AAC comprising the payload (such as HPV antigen and adjuvant) displays different characteristics compared to an anucleate cell comprising a payload introduced by other delivery methods (such as hemolytic loading or electroporation.

In some embodiments, the half-life of the AAC following administration to a mammal is decreased compared to a half-life of the input anucleate cell following administration to the mammal. In some embodiments, the hemoglobin content of the AAC is decreased compared to the hemoglobin content of the input anucleate cell. In some embodiments, ATP production of the AAC is decreased compared to ATP production of the input anucleate cell. In some embodiments, the AAC exhibits a spherical morphology. In some embodiments, the AAC is an erythrocyte and wherein the AAC has a reduced biconcave shape compared to the input anucleate cell. In some embodiments, the AAC is a red blood cell ghost. In some embodiments, the AACs prepared by the process have greater than about 1.5 fold more phosphatidylserine on its surface compared to the input anucleate cell. In some embodiments, a population profile of AACs prepared by the process exhibits higher average phosphatidylserine levels on the surface compared to the input anucleate cells. In some embodiments, at least 50% of the population profile of AACs prepared by the process exhibits higher phosphatidylserine levels on the surface compared to the input anucleate cells. In some embodiments, the AAC exhibits preferential uptake in a tissue or cell compared to the input anucleate cell. In some embodiments, the AAC exhibits preferential uptake in phagocytic cells and/or antigen presenting cells compared to the input anucleate cell. In some embodiments, the AAC is modified to enhance uptake in a tissue or cell compared to the input anucleate cell. In some embodiments, the AAC is modified to enhance uptake in phagocytic cells and/or antigen presenting cells compared to an unmodified AAC. In some embodiments, the phagocytic cells and/or antigen presenting cells comprise one or more of a dendritic cell or macrophage. In some embodiments, the tissue or cell comprises one or more of liver or spleen. In some embodiments, the AAC comprises CD47 on its surface.

In some embodiments of the above method for generating an AAC, the constriction is contained within a microfluidic channel. In some embodiments, the microfluidic channel comprises a plurality of constrictions. In some embodiments, the plurality of constrictions is arranged in series and/or in parallel. In some embodiments, the constriction is between a plurality of micropillars; between a plurality of micropillars configured in an array; or between one or more movable plates. In some embodiments, the constriction is a pore or contained within a pore. In some embodiments, the pore is contained in a surface. In some embodiments, the surface is a filter. In some embodiments, the surface is a membrane. In some embodiments, the constriction size is a function of the diameter of the input anucleate cell in suspension. In some embodiments, the constriction size is about 10%, about 20%, about 30%, about 40%, about 50%, about 60%, or about 70% of the diameter of the input anucleate cell in suspension. In some embodiments, the constriction has a width of about 0.25 μm to about 4 μm. In some embodiments, the constriction has a width of about 4 μm, 3.5 μm, about 3 μm, about 2.5 μm, about 2 μm, about 1.5 μm, about 1 μm, about 0.5 μm, or about 0.25 μm. In some embodiments, the constriction has a width of about 2.2 μm. In some embodiments, the input anucleate cells are passed through the constriction under a pressure ranging from about 10 psi to about 90 psi. In some embodiments, said cell suspension is contacted with the antigen before, concurrently, or after passing through the constriction.

In some embodiments, wherein the AAC comprising the payload (e.g. HPV antigen, or HPV antigen and an adjuvant) is prepared from an input anucleate cell, the AAC having one or more of the following properties: (a) a circulating half-life in a mammal is decreased compared to the input anucleate cell, (b) decreased hemoglobin levels compared to the input anucleate cell, (c) spherical morphology, (d) increased surface phosphatidylserine levels compared to the input anucleate cell, or (e) reduced ATP production compared to the input anucleate cell.

In some embodiments, the input anucleate cell is a mammalian cell. In some embodiments, the input anucleate cell is human cell. In some embodiments, the input anucleate cell is a red blood cell or a platelet. In some embodiments, the red blood cell is an erythrocyte or a reticulocyte.

In some embodiments, the circulating half-life of the AAC in a mammal is decreased compared to the input anucleate cell. In some embodiments, the circulating half-life in the mammal is decreased by more than about 50%, about 60%, about 70%, about 80%, about 90%, about 95%, about 96%, about 97%, about 98%, or about 99% compared to the input anucleate cell.

In some embodiments, the input anucleate cell is a human cell and wherein the circulating half-life of the AAC is less than about 1 minute, about 2 minutes, about 5 minutes, about 10 minutes, about 15 minutes, about 30 minutes, about 1 hour, about 6 hours, about 12 hours, about 1 day, about 2 days, about 3 days, about 4 days, about 5 days, about 10 days, about 25 days, about 50 days, about 75 days, about 100 days, about 120 days.

In some embodiments, the input anucleate cell is a red blood cell, wherein the hemoglobin levels in the AAC are decreased compared to the input anucleate cell. In some embodiments, the hemoglobin levels in the AAC are decreased by at least about 10%, about 20%, about 30%, about 40%, about 50%, about 60%, about 70%, about 80%, about 90%, about 99% or about 100% compared to the input anucleate cell. In some embodiments, the hemoglobin levels in the AAC are about 1%, about 5%, about 10%, about 20%, about 30%, about 40%, or about 50% the level of hemoglobin in the input anucleate cell.

In some embodiments, the input anucleate cell is an erythrocyte and wherein the AAC is spherical in morphology. In some embodiments, the input anucleate cell is an erythrocyte and wherein the AAC has a reduced biconcave shape compared to the input anucleate cell.

In some embodiments, the input anucleate cell is a red blood cell or an erythrocyte and wherein the AAC is a red blood cell ghost (RBC ghost).

In some embodiments, the AAC comprises CD47 on its surface.

In some embodiments, the AAC has increased surface phosphatidylserine levels compared to the input anucleate cell. In some embodiments, the AACs prepared by the process has greater than about 1.5 fold more phosphatidylserine on its surface compared to the input anucleate cell. In some embodiments, the AAC has about 10%, about 20%, about 30%, about 40%, about 50%, about 60%, about 70%, about 80%, about 90%, about 99%, about 100% or more than about 100% more phosphatidylserine on its surface compared to the input anucleate cell. In some embodiments, the level of phosphatidylserine on the surface of the AAC is determined by measuring the level of annexin staining (e.g., annexin V staining) on the surface of the AAC.

In some embodiments, the AAC has reduced ATP production compared to the input anucleate cell. In some embodiments, the AAC produces ATP at less than about 1%, about 5%, about 10%, about 20%, about 30%, about 40%, or about 50% the level of ATP produced by the input anucleate cell. In some embodiments, the AAC does not produce ATP.

In some embodiments, the AAC exhibits enhanced uptake in a tissue or cell compared to the input anucleate cell. In some embodiments, the AAC exhibits preferential uptake in liver or spleen or by a phagocytic cell or an antigen-presenting cell compared to the uptake of the input anucleate cell.

In some embodiments, the AAC is further modified to enhance uptake in a tissue or cell compared to the input anucleate cell. In some embodiments, the AAC is further modified to enhance uptake in liver or spleen or by a phagocytic cell or an antigen-presenting cell compared to the uptake of the input anucleate cell.

In some embodiments, wherein the AAC exhibits enhanced uptake in liver or spleen or by a phagocytic cell and/or an antigen-presenting cell, internalization of the AAC results in increased expression of maturation markers of the phagocytic cell or the antigen-presenting cell. In some embodiments, the phagocytic cell and/or the antigen-presenting cell is a monocyte-derived dendritic cell (MODC). In some embodiments, the maturation marker is one or more of CD80, CD86, CD83, and MHC-II. In some embodiments, the expression of one or more of CD80, CD86, CD83, and MHC-II is increased in the phagocytic cell and/or the antigen-presenting cell contacted with a AAC comprising a HPV antigen by at least about any one of: 10%, 20%, 50%, 80%, 100%, 2-fold, 5-fold, 10-fold, 20-fold, 50-fold, 100-fold, 1000-fold, 10000-fold or more compared to a phagocytic cell and/or an antigen-presenting cell not contacted with a AAC comprising a HPV antigen. In some embodiments, the expression of one or more of CD80, CD86, CD83, and MHC-II is increased in the phagocytic cell and/or the antigen-presenting cell contacted with a AAC comprising a HPV antigen by at least about any one of: 10%, 20%, 50%, 80%, 100%, 2-fold, 5-fold, 10-fold, 20-fold, 50-fold, 100-fold, 1000-fold, 10000-fold or more compared to a phagocytic cell and/or an antigen-presenting cell contacted with the input anucleate cell.

In some embodiments, wherein the AAC comprising an HPV antigen, or a HPV antigen and adjuvant exhibits enhanced uptake in liver or spleen or by a phagocytic cell and/or an antigen-presenting cell, internalization of the AAC results in increased presentation of the at least one HPV antigen comprised within the AAC. In some embodiments, the presentation of the at least one HPV antigen is increased in the phagocytic cell and/or the antigen-presenting cell contacted with a AAC comprising a HPV antigen by at least about any one of: 10%, 20%, 50%, 80%, 100%, 2-fold, 5-fold, 10-fold, 20-fold, 50-fold, 100-fold, 1000-fold, 10000-fold or more compared to a phagocytic cell and/or an antigen-presenting cell contacted with corresponding anucleate cells comprising the same HPV antigen introduced by other delivery methods (such as but not limited to hemolytic loading).

In some embodiments, wherein the AAC comprising an HPV antigen, or a HPV antigen and adjuvant exhibits enhanced uptake in liver or spleen or by a phagocytic cell and/or an antigen-presenting cell, internalization of the AAC results in increased ability of the phagocytic cell and/or the antigen-presenting cell to induce an antigen-specific immune response. In some embodiments, the antigen-specific immune response mediated by the phagocytic cell and/or the antigen-presenting cell contacted with a AAC comprising the at least one HPV antigen and adjuvant is increased by at least about any one of: 10%, 20%, 50%, 80%, 100%, 2-fold, 5-fold, 10-fold, 20-fold, 50-fold, 100-fold, 1000-fold, 10000-fold or more compared to a phagocytic cell and/or an antigen-presenting cell contacted with the input anucleate cells. In some embodiments, the antigen-specific immune response mediated by the phagocytic cell and/or the antigen-presenting cell contacted with a AAC comprising the at least one HPV antigen and adjuvant is increased by at least about any one of: 10%, 20%, 50%, 80%, 100%, 2-fold, 5-fold, 10-fold, 20-fold, 50-fold, 100-fold, 1000-fold, 10000-fold or more compared to a phagocytic cell and/or an antigen-presenting cell contacted with the anucleate cells comprising the same HPV antigen introduced by other delivery methods (such as but not limited to hemolytic loading). In some embodiments, the antigen-specific immune response is an antigen-specific CD4+ T cell response. In some embodiments, the antigen-specific immune response is an antigen-specific CD8⁺ T cell response.

In some embodiments, the individual is positive for HLA-A*02, HLA-A*01, HLA-A*03, HLA-A*24, HLA-A*11, HLA-A*26, HLA-A*32, HLA-A*31, HLA-A*68, HLA-A*29, HLA-A*23, HLA-B*07, HLA-B*44, HLA-B*08, HLA-B*35, HLA-B*15, HLA-B*40, HLA-B*27, HLA-B*18, HLA-B*51, HLA-B*14, HLA-B*13, HLA-B*57, HLA-B*38, HLA-C*07, HLA-C*04, HLA-C*03, HLA-C*06, HLA-C*05, HLA-C*12, HLA-C*02, HLA-C*01, HLA-C*08, and/or HLA-C*16.

In some embodiments according to any one of the methods, compositions, or uses described herein, the phagocytes are human cells with a haplotype of HLA-A*02, HLA-A*01, HLA-A*03, HLA-A*24, HLA-A*11, HLA-A*26, HLA-A*32, HLA-A*31, HLA-A*68, HLA-A*29, HLA-A*23, HLA-B*07, HLA-B*44, HLA-B*08, HLA-B*35, HLA-B*15, HLA-B*40, HLA-1B*27, HLA-B*18, HLA-1B*51, HLA-1B*14, HLA-1B*13, HLA-1B*57, HLA-1B*38, HLA-C*07, HLA-C*04, HLA-C*03, HLA-C*06, HLA-C*05, HLA-C*12, HLA-C*02, HLA-C*01, HLA-C*08, and/or HLA-C*16. In some embodiments, the antigen presenting cells are human cells with a haplotype of HLA-A*02, HLA-A*11, HLA-B*07, or HLA-C*08. In some embodiments, HPV antigens presented by the phagocytes and/or antigen presenting cells described herein are comprised of an HLA-A2-specific epitope. In some embodiments, HPV antigens presented by the phagocytes and/or antigen presenting cells described herein are comprised of an HLA-A11-specific epitope. In some embodiments, HPV antigens presented by the phagocytes and/or antigen presenting cells described herein are comprised of an HLA-B7-specific epitope. In some embodiments, HPV antigens presented by the phagocytes and/or antigen presenting cells described herein are comprised of an HLA-C8-specific epitope.

In some embodiments, the method comprises administering AACs comprising the at least one HPV antigen and adjuvant to the individual, wherein the AACs are internalized by phagocytic cells and/or antigen-presenting cell. In some embodiments, wherein the AACs are internalized by phagocytic cells and/or antigen-presenting cell, internalization of the AAC results in increased expression of maturation markers of the phagocytic cell or the antigen-presenting cell. In some embodiments, the phagocytic cell and/or the antigen-presenting cell is a monocyte-derived dendritic cell (MODC). In some embodiments, the maturation marker is one or more of CD80, CD86, CD83, and MHC-II. In some embodiments, the expression of one or more of CD80, CD86, CD83, and MHC-II is increased in the phagocytic cell and/or the antigen-presenting cell contacted with a AAC comprising a HPV antigen by at least about any one of: 10%, 20%, 50%, 80%, 100%, 2-fold, 5-fold, 10-fold, 20-fold, 50-fold, 100-fold, 1000-fold, 10000-fold or more compared to a phagocytic cell and/or an antigen-presenting cell not contacted with a AAC comprising a HPV antigen. In some embodiments, the expression of one or more of CD80, CD86, CD83, and MHC-II is increased in the phagocytic cell and/or the antigen-presenting cell contacted with a AAC comprising a HPV antigen by at least about any one of: 10%, 20%, 50%, 80%, 100%, 2-fold, 5-fold, 10-fold, 20-fold, 50-fold, 100-fold, 1000-fold, 10000-fold or more compared to a phagocytic cell and/or an antigen-presenting cell contacted with the input anucleate cell.

In some embodiments, the input anucleate cell was not (a) heat processed, (b) chemically treated, and/or (c) subjected to hypotonic or hypertonic conditions during the preparation of the AACs. In some embodiments, osmolarity was maintained during preparation of the AAC from the input anucleate cell. In some embodiments, the osmolarity was maintained between about 200 mOsm and about 600 mOsm. In some embodiments, the osmolarity was maintained between about 200 mOsm and about 400 mOsm.

### Systems and Kits

In some aspects, the invention provides a system comprising one or more of the constriction, an anucleate cell suspension, HPV antigens or adjuvants for use in the methods disclosed herein. The system can include any embodiment described for the methods disclosed above, including microfluidic channels or a surface having pores to provide cell-deforming constrictions, cell suspensions, cell perturbations, delivery parameters, compounds, and/or applications etc. In some embodiment, the cell-deforming constrictions are sized for delivery to anucleate cells. In some embodiments, the delivery parameters, such as operating flow speeds, cell and compound concentration, velocity of the cell in the constriction, and the composition of the cell suspension (e.g., osmolarity, salt concentration, serum content, cell concentration, pH, etc.) are optimized for maximum response of a compound for suppressing an immune response or inducing tolerance.

Also provided are kits or articles of manufacture for use in treating individuals with a cancer associated with HPV. In some embodiments, the kit comprises an AAC comprising intracellularly a mutated antigen and intracellularly an adjuvant. In some embodiments, the kit comprises one or more of the constriction, an anucleate cell suspension, HPV antigens or adjuvants for use in generating AACs for use in treating an individual with a disease associated with HPV, such as cancer. In some embodiments, the kits comprise the compositions described herein (e.g. a microfluidic channel or surface containing pores, cell suspensions, and/or compounds) in suitable packaging. Suitable packaging materials are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The invention also provides kits comprising components of the methods described herein and may further comprise instructions for performing said methods treat an individual with a cancer associated with HPV and/or instructions for introducing a HPV antigen and an adjuvant into an anucleate cell. The kits described herein may further include other materials, including other buffers, diluents, filters, needles, syringes, and package inserts with instructions for performing any methods described herein; e.g., instructions for treating an individual with a cancer associated with HPV or instructions for generating AACs to contain intracellularly a HPV antigen and intracellularly an adjuvant.

### Exemplary Embodiments

Embodiment 1. A method for treating a human papilloma virus (HPV)-associated cancer in an individual, the method comprising administering an effective amount of a composition comprising activating antigen carriers (AACs) to the individual wherein the effective amount is about 0.5×10⁸ AACs/kg to about 1×10⁹ AACs/kg, and wherein the AACs comprise at least one HPV antigen and an adjuvant delivered intracellularly.

Embodiment 2. A method for treating a human papilloma virus (HPV)-associated cancer in an individual, the method comprising:

administering an effective amount of a composition comprising activating antigen carriers (AACs) to the individual, wherein the AACs comprise at least one HPV antigen and an adjuvant delivered intracellularly, and

administering an effective amount of an antagonist of CTLA-4 and/or an antagonist of PD-1/PD-L1 to the individual.

Embodiment 3. The method of embodiment 2, wherein the antagonist of CTLA4 is an antibody that binds CTLA4.

Embodiment 4. The method of embodiment 2 or 3, wherein the antagonist of PD-1/PD-L1 is an antibody that binds PD-1 or an antibody that binds PD-L1.

Embodiment 5. The method of embodiment 3 or 4, wherein an antibody that binds CTLA-4 and an antibody that binds PD-1 are administered to the individual.

Embodiment 6. The method of any one of embodiments 3-5, wherein the antibody that binds CTLA-4 is ipilimumab.

Embodiment 7. The method of any one of embodiments 4-6, wherein the antibody that binds PD-1 is nivolumab.

Embodiment 8. The method of any one of embodiments 4-6, wherein the antibody that binds PD-1 is pembrolizumab.

Embodiment 9. The method of any one of embodiments 4-6, wherein an antibody that binds CTLA-4 is administered to the individual and an antibody that binds PD-L1 is administered to the individual.

Embodiment 10. The method of any one of embodiments 4 and 9, wherein the antibody that binds PD-L1 is atezolizumab.

Embodiment 11. The method of any one of embodiments 1-10, wherein the at least one HPV antigen is a HPV-16 antigen or a HPV-18 antigen.

Embodiment 12. The method of any one of embodiments 1-11, wherein the at least one HPV antigen comprises a peptide derived from HPV E6 and/or E7.

Embodiment 13. The method of any one of embodiments 1-12, wherein the at least one HPV antigen comprises an HLA-A2-restricted peptide derived from HPV E6 and/or E7.

Embodiment 14. The method of embodiment 13, wherein the HLA-A2-restricted peptide comprises the amino acid sequence of any one of SEQ ID NOs:1-4.

Embodiment 15. The method of any one of embodiments 1-12, wherein the at least one HPV antigen comprises the amino acid sequence of any one of SEQ ID NOs:18-25.

Embodiment 16. The method on any one of embodiments 1-12, wherein the AACs comprise an antigen comprising the amino acid sequence of SEQ ID NO:19 and an antigen comprising the amino acid sequence of SEQ ID NO:23.

Embodiment 17. The method of any one of embodiments 1-16, wherein the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist.

Embodiment 18. The method of embodiment 17, wherein the adjuvant is a CpG 7909 oligodeoxynucleotide (ODN).

Embodiment 19. The method of any one of embodiments 1-18, where the individual is human.

Embodiment 20. The method of any one of embodiments 1-19, wherein the individual is positive for HLA-A*02.

Embodiment 21. The method of any one of embodiments 1-20, where the AACs are autologous or allogeneic to the individual.

Embodiment 22. The method of any one of embodiments 1-21, wherein the HPV-associated cancer is a current, locally advanced or metastatic cancer.

Embodiment 23. The method of any one of embodiments 1-22, wherein the HPV-associated cancer is head and neck cancer, cervical cancer, anal cancer or esophageal cancer.

Embodiment 24. The method of any one of embodiments 1-23, wherein the composition comprising AACs are administered intravenously.

Embodiment 25. The method of any one of embodiments 2-24, wherein the antagonist of CTLA-4 and/or antagonist of PD-1/PD-L1 is administered intravenously, orally, or subcutaneously.

Embodiment 26. The method of any one of embodiments 3-25, wherein the antibody that binds CTLA-4 and/or the antibody that binds PD-1 and/or the antibody that binds PD-L1 is administered intravenously.

Embodiment 27. The method of any one of embodiments 1-26, wherein the effective amount of AACs comprising the at least one HPV antigen and the adjuvant is about 0.5×10⁸ AACs/kg to about 7.5×10⁸ AACs/kg.

Embodiment 28. The method of any one of embodiments 1-27, wherein the effective amount of AACs comprising the at least one HPV antigen and the adjuvant is about 0.5×10⁸ AACs/kg to about 1×10⁹ AACs/kg.

Embodiment 29. The method of any one of embodiments 1-28, wherein the effective amount of AACs comprising the at least one HPV antigen and the adjuvant is about 0.5×10⁸ AACs/kg, about 2.5×10⁸ AACs/kg, about 5×10⁸ AACs/kg, or about 7.5×10⁸ AACs/kg.

Embodiment 30. The method of any one of embodiments 6-29, wherein the effective amount of ipilimumab is about 1 mg/kg to about 3 mg/kg.

Embodiment 31. The method of any one of embodiments 7 and 11-30, wherein the effective amount of nivolumab is about 360 mg.

Embodiment 32. The method of any one of embodiments 10-30, wherein the effective amount of atezolizumab is about 1200 mg.

Embodiment 33. The method of any one of embodiments 1-32, wherein the composition comprising the AACs is delivered on day 1 of a three-week cycle.

Embodiment 34. The method of any one of embodiments 1-33, wherein the composition comprising the AACs is further administered on day 2 of a first three-week cycle.

Embodiment 35. The method of embodiment 33 or 34, wherein about 0.5×10⁸ cells/kg to about 1×10⁹ cells/kg are administered on day 1 of each three-week cycle.

Embodiment 36. The method of any one of embodiments 33-35, wherein about 0.5×10⁸ cells/kg, about 2.5×10⁸ cells/kg, about 5.0×10⁸ cells/kg, or about 7.5×10⁸ cells/kg are administered on day 1 of each three-week cycle.

Embodiment 37. The method of any one of embodiments 33-36, wherein about 0.5×10⁸ cells/kg to about 1×10⁹ cells/kg are administered on day 2 of each three-week cycle.

Embodiment 38. The method of any one of embodiments embodiment 33-37, wherein about 0.5×10⁸ cells/kg, about 2.5×10⁸ cells/kg, about 5.0×10⁸ cells/kg, or about 7.5×10⁸ cells/kg are administered on day 2 of the first three-week cycle.

Embodiment 39. The method of any one of embodiments 33-38, wherein an antibody that binds CTLA-4 and/or an antibody that binds PD-1 and/or an antibody that binds PD-L1 is administered once per three-week cycle.

Embodiment 40. The method of any one of embodiments 33-39, wherein an antibody that binds CTLA-4 is administered once per two three-week cycles.

Embodiment 41. The method of any one of embodiments 33-40, wherein an antibody that binds CTLA-4 is administered on day 1 of each three-week cycle.

Embodiment 42. The method of any one of embodiments 39-41, wherein the antibody that binds CTLA-4 is ipilimumab, wherein the ipilimumab is administered at a dose of about 3 mg/kg.

Embodiment 43. The method of any one of embodiments 39-42, wherein the antibody that binds PD-1 is administered on day 8 of the first three-week cycle and day 1 of each subsequent cycle.

Embodiment 44. The method of embodiment 43, wherein the antibody that binds PD-1 is nivolumab, wherein the nivolumab is administered at a dose of about 360 mg.

Embodiment 45. The method of any one of embodiments 39-44, wherein the antibody that binds CTLA-4 is ipilimumab, wherein the ipilimumab is administered on day 1 of the first three-week cycle of two three-week cycles at a dose of about 1 mg/kg and the antibody that binds PD-1 is administered on day 8 of the first three-week cycle and day 1 of each subsequent cycle at a dose of about 360 mg.

Embodiment 46. The method of any one of embodiments 33-39, wherein an antibody that binds PD-L1 is administered on day 8 of the first three-week cycle and day 1 of each subsequent cycle.

Embodiment 47. The method of embodiment 46, wherein the antibody that binds PD-L1 is atezolizumab, wherein the atezolizumab is administered at a dose of about 1200 mg.

Embodiment 48. The method of any one of embodiments 1-47, wherein the composition comprising PBMCs is administered to the individual for at least about three months, six months, nine months or one year.

Embodiment 49. The method of any one of embodiments 1-48, wherein the composition comprising AACs comprises about 1×10⁹ AACs to about 1×10¹⁰ AACs in a cryopreservation medium.

Embodiment 50. The method of any one of embodiments 1-49, wherein the composition comprising AACs comprises about 7×10⁹ PBMCs in about 10 mL of a cryopreservation medium.

Embodiment 51. The method of embodiment 49 or 50, wherein the cryopreservation medium is Cryostor® CS2.

Embodiment 52. The method of any one of embodiments 1-51, wherein the AACs comprising the at least one HPV antigen and an adjuvant are prepared by a process comprising:

a) passing a cell suspension comprising a population of input anucleate through a cell-deforming constriction, wherein a diameter of the constriction is a function of a diameter of the input anucleate cells in the suspension, thereby causing perturbations of the input anucleate cells large enough for the at least one HPV antigen and the adjuvant to pass through to form perturbed input anucleate cells; and

b) incubating the population of perturbed input anucleate cells with the at least one HPV antigen and the adjuvant for a sufficient time to allow the antigen to enter the perturbed input anucleate cells, thereby generating the AACs comprising the at least one HPV antigen and the adjuvant.

Embodiment 53. The method of embodiment 52, wherein the diameter of the constriction is about 1.6 μm to about 2.4 μm or about 1.8 μm to about 2.2 μm.

Embodiment 54. The method of embodiment 52 or 53, wherein the input anucleate cell is a red blood cell.

Embodiment 55. The method of any one of embodiments 52-54, wherein the at least one HPV antigen comprises a peptide derived from HPV E6 and a peptide derived from HPV E7.

Embodiment 56. The method of any one of embodiments 52-55, wherein the at least one HPV antigen comprises the amino acid sequence of any one of SEQ ID NOs:1-4.

Embodiment 57. The method of any one of embodiments 52-55, wherein the at least one HPV antigen comprises the amino acid sequence of any one of SEQ ID NOs:18-25.

Embodiment 58. The method of any one of embodiments 52-55, wherein the AACs comprise an antigen comprising the amino acid sequence of SEQ ID NO:19 and an antigen comprising the amino acid sequence of SEQ ID NO:23.

Embodiment 59. The method of any one of embodiments 52-58, wherein the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist.

Embodiment 60. The method of embodiment 59, wherein the adjuvant is a CpG 7909 oligodeoxynucleotide (ODN).

### EXAMPLES

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### Example 1. Phase I Study of Safety and Tolerability of SQZ-AAC-HPV

A Phase 1 open-label, multicenter study of the safety and tolerability, antitumor activity, and immunogenic and pharmacodynamic effects of SQZ-AAC-HPV as monotherapy and in combination with (1) ipilimumab, (2) nivolumab, and (3) nivolumab plus ipilimumab, in HLA A*02+ patients with recurrent, locally advanced or metastatic HPV16+ solid tumors is conducted.

SQZ-AAC-HPV is a red blood cell (RBC)-derived product of activating antigen carriers (AAC) used as a treatment for human papillomavirus (HPV) strain 16 positive (HPV16+) cancer in human leukocyte antigen (HLA) serotype within the HLA-A serotype group positive (HLA-A*02+) patients. SQZ-AAC-HPV consists of autologous RBCs processed with HLA-A*02-restricted E6 and E7 epitopes of HPV16 and the adjuvant, polyinosinic-polycytidylic acid (poly I:C), which are delivered cytosolically during manufacturing.

E6 SLP:
(SEQ ID NO: 19)
QLCTELQTTIHDIILECVYCKQQLL
E7 SLP:
(SEQ ID NO: 23)
QLCTELQTYMLDLQPETTYCKQQLL

The process starts with the specific patient at a clinical site where whole blood is collected and then shipped to the manufacturing site. At the manufacturing site, the platelets and white blood cells are removed and the E6 and E7 epitopes, along with the poly I:C adjuvant, are delivered into the cells using the Cell Squeeze technology. As a result of the Cell Squeeze technology, there is an increase in phosphatidylserine on the surface of the AAC relative to the starting RBC. The resultant cells are the AAC-HPV drug substance. The AAC-HPV drug substance is washed with cryopreservation media, subsequently formulated into SQZ-AAC-HPV autologus drug product, and cryopreserved. SQZ-PBMC-HPV drug substances consists of autologous PBMCs that have synthetic long peptides (SLPs) containing HLA-A*02-restricted E6 and E7 epitopes of HPV16 delivered cytosolically during the manufacturing process.

### Overview

The study population consists of patients who are HLA-A*02+ with advanced-stage HPV16+ solid tumors (head and neck, cervical cancer, and other tumor types). HLA A*02+ status and HPV16+ tumor status is confirmed via laboratory reports, and all eligibility criteria must be met, prior to the patient's blood collection for manufacture of autologous blood product. Patients with locally confirmed HPV16+ status may have central confirmation done from the fresh tumor biopsy collected at Screening if documentation of laboratory accreditation is deemed by the Sponsor to be insufficient.

Eligible patients will undergo a single blood collection at the study sites for manufacture of autologous drug product. At least 200 mL of whole blood is drawn for this purpose. This blood collection is sent to a contract manufacturer for manufacture of each patient's personalized autologous cellular therapy. Frozen vials of SQZ AAC HPV are then sent to the study sites for administration.

This study is conducted in 2 parts, with Part 1 consisting of a dose escalation to determine the safety profile, preliminary efficacy, and RP2D of SQZ-AAC-HPV monotherapy. Part 2 of the study will evaluate the safety and preliminary efficacy of SQZ-AAC-HPV when combined with immune checkpoint inhibitors, the Combination Safety Phase.

In all cohorts, SQZ AAC-HPV is administered at 3-week intervals for a maximum of 1 year or until the SQZ-AAC-HPV supply is exhausted or treatment discontinuation criteria are met, whichever comes first.

All patients in Part 1 and Part 2 are observed for at least 4 hours after each administration of SQZ AAC HPV. In addition, the first 2 patients in each cohort undergo a minimum 23 hours of observation following the first administration of SQZ AAC-HPV.

Tumor assessments is performed throughout the study per RECIST 1.1 and iRECIST until disease progression, unacceptable toxicity, withdrawal of consent, death, or for 2 years from the date of the first administration of SQZ AAC HPV, whichever occurs first. Patients who experience disease progression per RECIST 1.1 may continue dosing if considered in their best interest by the treating Investigator to allow for confirmation of disease progression; i.e., iCPD according to iRECIST (Seymour et al, 2017).

After the last dose of investigational product, follow-up visits occur to monitor safety and tolerability and evaluate overall survival.

### Part 1: Escalation Phase (SQZ-AAC-HPV Monotherapy)

Planned dose cohorts for the Escalation Phase are shown in Table 1. While the traditional 3+3 design is intended to assess safety and tolerability, it may be prudent to treat up to a total of 12 additional patients in a cohort to further investigate safety and tolerability, immunogenic effects, and antitumor activity. There will be a maximum of 12 patients per cohort in this modified 3+3 design.

TABLE 1
Summary of Monotherapy Cohorts Planned During the Escalation Phase

| | Dose of SQZ-AAC-HPV | Potential Number of Patients/ |
|---|---|---|
| Cohort | AACs/kg ^{a,b} | Cohort based on DLT |
| 1a | 0.5 × 10⁸ | 3-12 |
| 1b | 2.5 × 10 ⁸ OR^{c} 5 × 10⁸ | 3-12 |
| 1c | Higher^{d} or lower dose | 3-12 |

^{a} Dosing with SQZ-AAC-HPV continues every 3 weeks until treatment discontinuation criteria are met, the SQZ-AAC-HPV supply is exhausted, or for a maximum of 1 year, whichever comes first.
^{b} In Cycle 1, patients will receive SQZ-AAC-HPV on Days 1 and 2.
^{c}If no safety signal is observed, the high dose level will be 5 × 10⁸ AAC/kg. If ≥ Grade 2 related non-PD associated toxicity is observed in 1 out of 3 or 2 out of 6 patients during the DLT period, the high dose level will be 2.5 × 10⁸ AAC/kg.
^{d}If a high dose of 2.5 × 10⁸ AAC/kg is chosen, a third cohort with 5 × 10⁸ AAC/kg may be opened, once the DLT assessment of 2.5 × 10⁸ AAC/kg is completed.

At least 2 monotherapy dose levels are tested. The low dose of SQZ-AAC-HPV will be 0.5×10⁸ AAC/kg. To ensure patients in Cohort 2 are exposed to the most immunogenic cell dose possible, the high dose level of SQZ-AAC-HPV is based on safety findings in Cohort 1. If no safety signal is observed (i.e., no ≥Grade 2 treatment-related toxicity) the high dose level is 5×10⁸ AAC/kg. If ≥Grade 2 related non-PD associated toxicity (or DLT) is observed in 1 out of 3 or 2 out of 6 patients during the DLT period, the high dose level is 2.5×10⁸ AAC/kg. If a high dose of 2.5×10⁸ AAC/kg is chosen, a third cohort with 5×10⁸ AAC/kg may be opened, once the DLT assessment of 2.5×10⁸ AAC/kg is completed. Following review of the available safety, efficacy, and pharmacodynamic data from patients in a given cohort, the SSC determines whether exploration of additional higher or lower single- or double antigen loading dose levels is warranted. In this case, the magnitude of the dose escalation or de-escalation will be determined by the SSC, based on the type and severity of TEAEs observed.

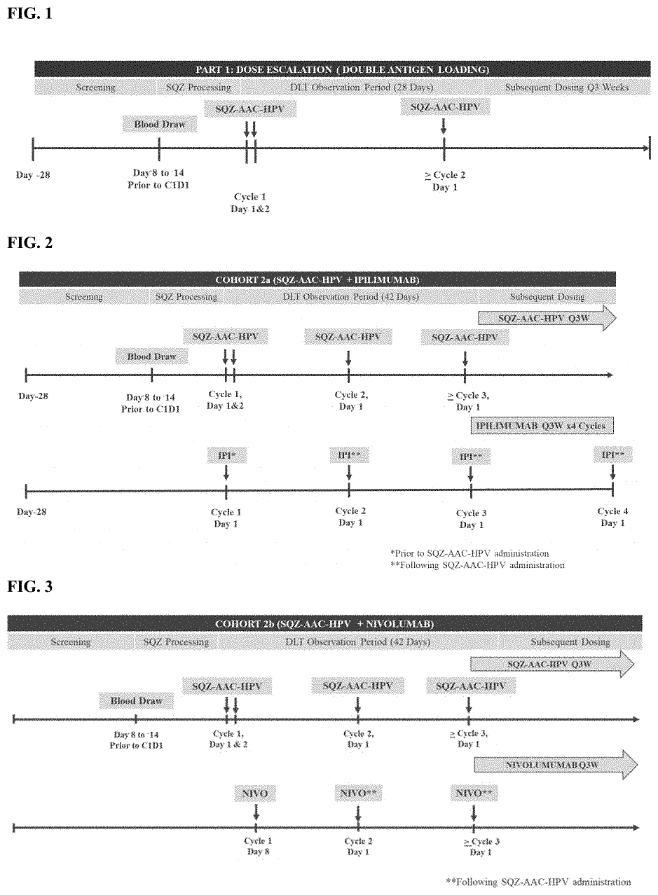

Patients receive SQZ-AAC-HPV on Days 1 and 2 in Cycle 1 and on Day 1 of each subsequent, 21-day cycle. In Part 1, the DLT observation period is 28 days (FIG. 1).

Patients are enrolled in a staggered manner across investigative sites, meaning no more than 1 patient in a cohort will receive the first administration of SQZ AAC-HPV within 1 week. Administration of SQZ AAC-HPV in subsequent cohorts will not begin until the SSC has reviewed available safety data and determined that dose escalation is warranted.

### Dose Escalation and RP2D Determination

While the traditional 3+3 design is intended to assess safety and tolerability, it may be prudent to treat up to 6 additional patients in a cohort to further investigate safety and tolerability, immunogenic effects, and antitumor activity. There are a maximum of 12 patients per cohort in this modified 3+3 design.

Dose escalation or increase in cohort size to 6 to 12 patients is considered after the first 3 patients at a given dose level have completed the DLT observation period and are found to be evaluable for safety upon review of safety data conducted by the SSC. The DLT observation period is defined as 28 days for Part 1.

If there are no DLTs observed in any of the first 3 enrolled patients at a given dose level through the DLT observation period, then the next higher dose level cohort may be opened for enrollment. If 1 of the first 3 patients experiences DLT, then 3 additional patients are enrolled (total of 6 evaluable patients at the same dose level). If ≥1 of the first 3 patients or ≥2 of 6 patients experience DLT, then no further dose escalation will be considered and this will be the maximum administered dose (MAD). The RP2D may be a previously evaluated, lower dose level; or an alternative intermediate dose level may be selected for further evaluation. The RP2D determination is made by the SSC based on safety data from at least 6 patients. The RP2D is further evaluated in Part 2 (Combination Safety Phase) of the study. Alternatively, the RP2D may be declared, based on pharmacodynamic assessment, where it is determined that the maximum biologic effect has been achieved, and that patients would not benefit from further dose escalation.

A patient will be considered non-evaluable if, for any reason other than safety, the patient is unable to complete the DLT observation period or if the pharmacodynamic assessments are insufficient to define the biological effect of study treatment. Patients in Part 1 considered non-evaluable may be replaced after consultation between the investigators and Sponsor.

Adverse events that develop after any administered dose is resolved to ≤Grade 2 at time of subsequent administrations. Similarly, adverse events of special interest (AESIs) that develop after any administered dose is resolved to <Grade 2 at time of subsequent administration. If, following the first administration in Cycle 1, these retreatment criteria are met, the second SQZ AAC-HPV administration is given during the ≥23-hour observation period (i.e., between 16 and 24 hours post first dose). Patients are observed for a minimum of 4 hours after the second antigen loading administration. The minimum interval between the 2 administrations is 16 hours.

Patients are monitored for the occurrence of DLTs for 28 days after the first dose of SQZ AAC HPV in monotherapy cohorts. Following the modified 3+3 rules, the minimum number of patients needed to confirm a cohort as safe with respect to DLTs is 0 DLTs in 3 patients, ≤1 DLT in 6 patients, ≤2 DLTs in 9 patients or ≤3 DLTs in 12 patients.

For the determination of the monotherapy RP2D regimen, the DLT assessment in all cohorts must be complete. The RP2D regimen is selected based on review of all available safety, tolerability, immunogenic, and other pharmacodynamic and antitumor data. The SSC reviews the data and make a recommendation to the DSMB, who are responsible for RP2D approval.

Once the RP2D regimen is defined, Part 2 (Combination Safety Phase) may be initiated.

### Part 2: Combination Safety Phase (SQZ-AAC-HPV+Checkpoint Inhibitor[s])

The SQZ-AAC-HPV dose evaluated during Combination Safety exploration is selected based on review of all available safety, tolerability, immunogenic, and other pharmacodynamic and antitumor data. The DSMB decides whether to select the SQZ-AAC-HPV monotherapy RP2D for the Combination Safety Phase or to start at a lower dose.

The cohorts are defined by the SQZ AAC HPV RP2D and the combination partner. SQZ AAC-HPV is administered in the RP2D in Cohorts 2a, 2b, and 2c.

Cohort 2a: SQZ-AAC-HPV (RP2D) plus ipilimumab (3 mg/kg every 3 weeks for a maximum of 4 doses if tolerability allows)

Cohort 2b: SQZ-AAC-HPV (RP2D) plus nivolumab (360 mg every 3 weeks)

Cohort 2c (contingent on the safety assessment of 6 patients each treated in Cohorts 2a and 2b): SQZ-AAC-HPV (RP2D) plus nivolumab (360 mg every 3 weeks) and ipilimumab (1 mg/kg every 6 weeks)

Enrollment in Part 2 begins with Cohorts 2a and 2b. Once 6 patients each in Cohorts 2a and 2b are enrolled and successfully complete the 42-day DLT evaluation period; i.e., <33% of patients experience DLT, then Cohort 2c opens for enrollment. Based on the available safety data from both cohorts, the SSC decides whether the SQZ-AAC-HPV dose regimen selected for Cohorts 2a and 2b is selected for Cohort 2c or whether to start at a lower dose regimen. If the SSC recommends starting Cohort 2c at a lower dose of SQZ-AAC-HPV, 6 patients are enrolled initially and at least 4 patients observed for 42 days. If the SSC deems the combination safe, with <33% of patients experiencing DLT, the dose of SQZ-AAC-HPV may be escalated to the full monotherapy RP2D and enrollment may continue until up to 12 patients have been enrolled if warranted.

Patients in the Part 2 Combination Safety cohorts receive SQZ-AAC-HPV on Days 1 and 2 of Cycle 1 and on Day 1 of each subsequent 21-day cycle. In each cohort, the first 2 patients completes Cycle 1 Day 8 before additional patients in the cohort can be treated in that cohort.

All patients are evaluated for safety and tolerability as well as preliminary evidence of antitumor response.

Cohort 2a—SQZ-AAC-HPV plus Ipilimumab

In Cycle 1, SQZ-AAC-HPV is administered IV in accordance with the RP2D determined in Part 1; i.e., either as double antigen loading on Days 1 and 2, or as a single antigen loading dose on Day 1. Ipilimumab, 3 mg/kg, is administered IV over 90 minutes, prior to SQZ AAC HPV on Day 1. In Cycles 2, 3, and 4, ipilimumab is given on Day 1 following the administration of SQZ-AAC-HPV. Ipilimumab is administered for a maximum of 4 cycles. SQZ-AAC-HPV is given in 3-week cycles until discontinuation criteria are met, the SQZ-AAC-HPV supply has been exhausted, or for up to 1 year, whichever comes first (FIG. 2).

Cohort 2b—SQZ-AAC-HPV plus Nivolumab

In Cycle 1, SQZ-AAC-HPV is administered IV in accordance with the RP2D determined in Part 1; i.e., either as double antigen loading on Days 1 and 2, or as a single antigen loading dose on Day 1. On Cycle 1 Day 8, nivolumab is administered at a dose of 360 mg IV, over 30 minutes, immediately following completion of the SQZ-AAC-HPV infusion. In subsequent cycles, SQZ AAC-HPV followed by nivolumab is administered on Day 1, every 3 weeks. Nivolumab may be given every 3 weeks for up to 2 years or until discontinuation criteria are met. SQZ-AAC-HPV is administered in 3-week cycles until discontinuation criteria are met, the SQZ-AAC-HPV supply has been exhausted, or for a maximum of 1 year, whichever comes first (FIG. 3).

Cohort 2c—SQZ-AAC-HPV plus Nivolumab plus Ipilimumab

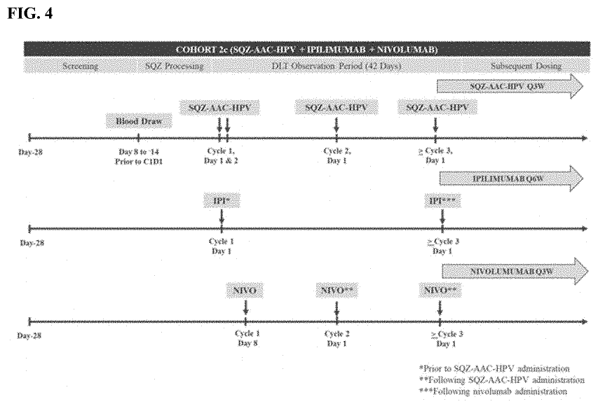

In Cycle 1, SQZ-AAC-HPV is administered IV in accordance with the RP2D based on the findings in Cohorts 2a and 2b. Of note, the SSC may determine that proceeding at a dose below the dose selected for Cohorts 2a and 2b or a modified dose regimen (e.g. as a single antigen loading dose on Day 1 only) is advised. Ipilimumab is administered IV at a dose of 1 mg/kg, over 30 minutes on Day 1, prior to SQZ-AAC-HPV. On Cycle 1, Day 8, nivolumab 360 mg IV is administered over 30 minutes. Nivolumab is given on Day 1 in subsequent, 3-week cycles, following administration of SQZ-AAC-HPV. Ipilimumab is administered every 6 weeks, following administration of SQZ-AAC-HPV and nivolumab in subsequent cycles (FIG. 4). Nivolumab and ipilimumab may be given for 2 years from Cycle 1 Day 1 until 1 of the criteria for treatment discontinuation are met. SQZ-AAC-HPV is administered in 3-week cycles until discontinuation criteria are met, the SQZ-AAC-HPV supply has been exhausted, or for a maximum of 1 year, whichever comes first.

If, due to an immune-mediated AE, a patient meets criteria for discontinuation of checkpoint inhibitors (according to Appendix E), and the investigator is unable to determine whether the event is related to nivolumab or ipilimumab, the patient discontinues both drugs, and may continue on SQZ-AAC-HPV.

For all cohorts in Part 2, the second SQZ-AAC-HPV administration on Cycle 1 Day 2 is given during the ≥23-hour observation. Adverse events that develop after any administered dose are resolved to ≤Grade 2 at time of subsequent administration. Similarly, AESIs that develop after any administered dose are resolved to <Grade 2 at time of subsequent administration. If these retreatment criteria are met, the second SQZ AAC HPV administration is given during the ≥23-hour observation period (i.e., between 16 and 24 hours post first dose). Patients are observed for a minimum of 4 hours after the second antigen loading administration. The minimum interval between the 2 administrations is 16 hours. In each cohort, the first 2 patients complete Cycle 1 Day 14 before additional patients in the cohort are treated.

Patients are monitored for the occurrence of DLTs for 42 days after the first dose of SQZ AAC-HPV in combination therapy cohorts.

In case of a DLT or other significant toxicity in individual patients, de-escalation to a lower SQZ AAC-HPV dose will occur. Following review of the available safety, efficacy, and pharmacodynamic data from patients in individual combination safety cohorts, the SSC may determine that double antigen loading is not advisable for 1 or more dose combinations. In this case, the SSC may recommend dropping the second (Cycle 1 Day 2) SQZ-AAC-HPV dose. Alternatively, the SSC may determine that a lower dose level for SQZ-AAC-HPV may explored (dose de-escalation). For instance, if DLT is observed in ≥33% of patients in individual combination safety cohorts, a cohort evaluating a lower SQZ-AAC-HPV level is opened and explored. Higher dose cohorts are denoted 2c, 2d, etc. and lower dose cohorts are denoted 2a-1, 2b-1, etc.

### Dosing Schedule and Study Duration

All patients undergo a single blood collection that is used for manufacture of autologous blood product prior to the start of treatment. Patients undergo this blood collection at the study sites; this typically occurs approximately 1 to 2 weeks prior to (Cycle 1 Day 1) the initial administration of SQZ AAC HPV. Scheduling of the first administration of SQZ AAC HPV takes into account site location and shipping logistics.

A cycle is defined as a treatment period of 21 days.

Patients receive SQZ AAC HPV at 3-week intervals for up to 1 year, until investigational product is exhausted, or until treatment discontinuation criteria are met, whichever comes first.

Accumulating clinical evidence indicates some subjects treated with immune system stimulating agents may reveal signs of progression of disease (by conventional response criteria) before demonstrating clinical objective responses and/or stable disease. Two hypotheses have been put forth to explain this phenomenon. First, enhanced inflammation within tumors could lead to an increase in tumor size, which would appear as enlarged index lesions and as newly visible small non-index lesions. Over time, both the malignant and inflammatory portions of the mass may then decrease, leading to overt signs of clinical improvement (Wolchok et al, 2009). Alternatively, in some individuals, the kinetics of tumor growth may initially outpace anti-tumor immune activity. With sufficient time, the anti-tumor activity will dominate and become clinically apparent. Thus, it is important to assess RECIST 1.1 and iRECIST in parallel at each time point

Patients may continue study therapy after initial RECIST 1.1-defined progression, and therefore allow for confirmation of disease progression according to iRECIST (Seymour et al, 2017) if the following criteria are met:

1. Investigator-assessed clinical benefit, and lack of rapid disease progression

2. Tolerance of study drug, as defined by the investigator

3. Stable performance status

4. Treatment beyond progression will not delay an imminent intervention to prevent serious consequence from rapidly progressing disease

5. Lack of complications of disease progression (e.g., CNS metastases)

The assessment of clinical benefit takes into account whether the patient is clinically deteriorating and unlikely to receive further benefit from continued treatment.

### Dose-Limiting Toxicity

A patient is considered evaluable for DLT assessment if he or she: 1) experiences a DLT during the DLT assessment period, regardless of the cell dose received; or 2) does not experience a DLT during the DLT assessment period after having received at least 70% of the intended dose of SQZ-AAC-HPV during the DLT assessment period. Patients who do not experience a DLT and yet received less than 70% of the intended SQZ-AAC-HPV dose during the DLT assessment period are not considered evaluable for DLT and are replaced.

Patients experiencing a DLT that is not an IRR are discontinued from the study. If, in the opinion of the Investigator and the Sponsor, it is in the patient's best interest to continue treatment on investigational product, then the subsequent treatment will be determined by the Investigator in consultation with the Sponsor. For IRRs, the premedication or rate of administration may be adjusted to enable the patient to remain on study.

A DLT is defined as an AE or clinically significant abnormal laboratory value assessed by the Principal Investigator and confirmed by the SSC as unrelated to disease, disease progression, intercurrent illness, concomitant medications/procedures, or environmental factors, but related to SQZ-AAC-HPV (either alone or in combination), occurring within either the first 28 days of treatment with monotherapy or the first 42 days of treatment with combination therapy, and which meets any of the pre-defined criteria as listed below using National Cancer Institute (NCI) Common Terminology Criteria for Adverse Events (CTCAE) version 5.0. Grading of CRS and neurotoxicity will use the American Society for Transplantation and Cellular Therapy (ASTCT) Consensus Grading, as referenced in Section 6.1.4 and Section 6.1.5, respectively.

Non-Hematologic Toxicity
Grade 4 or Grade 5.
Grade 3 toxicity that does not resolve to ≤Grade 1 or Baseline within 7 days despite optimal supportive care, except for Grade 3 CRS or neurotoxicity that does not resolve to ≤Grade 2 within 24 hours.
Grade 3 laboratory value that persists >7 days and requires medical intervention.
>Grade 3 hepatic toxicity lasting for >48 hours with the following exception: For patients with Grade 2 aspartate aminotransferase (AST), alanine aminotransferase (ALT), and/or alkaline phosphatase abnormalities at Baseline, only an increase to >8× upper limit of normal (ULN) lasting >48 hours will be considered a DLT.
Liver tests abnormalities meeting Hy's law criteria.

Hematologic Toxicity
Any Grade 5 toxicity.
Any Grade 4 anemia.
Any ≥Grade 3 febrile neutropenia.
≥Grade 4 neutropenia (absolute neutrophil count <500/μL) lasting >7 days.
≥Grade 4 thrombocytopenia (<25,000/μL).
≥Grade 3 thrombocytopenia (<50,000/μL) lasting >7 days associated with clinically significant bleeding.

TEAEs at least possibly related to SQZ-AAC-HPV (either alone or in combination) that result in permanent discontinuation or a delay of >14 days of Cycle 2 Day 1 of scheduled SQZ AAC HPV administration.

Any other event that, in the judgement of the Investigator and Sponsor, is considered to be a DLT.

The following events are not considered a DLT:

Isolated Grade 3 lipase values that are not accompanied by >Grade 3 amylase values or clinical symptoms or radiographic evidence of pancreatitis.

Grade 3 CRS that improves to ≤Grade 2 within 24 hours with or without symptomatic treatment.

Grade 3 skin rash that resolves to ≤Grade 2 within 7 days with or without appropriate supportive care.

Immediate hypersensitivity reactions occurring within 2 hours of cell product administration that are reversible to ≤Grade 2 with 24 hours.

Grade 1 or Grade 2 electrolyte abnormalities that are corrected within 72 hours without clinical sequelae.

Alopecia.

A Grade 3 IRR that can be adequately managed with the addition of premedication or modification of the rate of administration is not be considered a DLT, unless these changes are considered applicable to all subsequent patients enrolled in the study based on the recommendations of the SSC. If the modification(s) applies to all subsequent patients, the cohort restarts for the DLT evaluation. The patient who experienced the Grade 3 infusion reaction may stay on study with modification to their premedication or the infusion rate.

If the maximum tolerated dose (MTD) is not reached in any cohort, additional cell dose levels or regimens may be tested. In the event of AEs covered by the definition of a DLT but unrelated to SQZ-AAC-HPV, the findings will be discussed by the SSC.

### Stopping Criteria for a Cohort and Stopping of Dose Escalation or Progression to Cohort and Termination of Study

The modified 3+3 rules define the ultimate decision to declare a cohort as safe. The minimum number of patients needed to confirm a cohort as safe is 3 patients with 0 DLTs, which can be increased up to 12 patients to confirm that a cohort is safe (i.e., <33% of patients with DLT; for instance, 6 patients with <2 DLTs, 9 patients with <3 DLTs, or 12 patients with <4 DLTs, whichever confirms the safety of the cohort). If none of the cohorts indicate that the MTD has been reached, additional cell dose levels or regimens may be tested. In the event of AEs covered by the definition of a DLT but unrelated to SQZ-AAC-HPV, the findings will be discussed by the SSC.

An AE that meets the definition of a DLT and occurring outside the DLT window will not be counted as a DLT but instead will be considered in the overall safety assessment of a given cohort and the selection of an RP2D regimen.

The cohort stopping rule is the occurrence of ≥3 DLTs in up to 12 patients (≥33%) receiving investigational product within the same dose cohort. If the stopping rule is triggered, the SSC may make 1 of the following recommendations:

Declare the prior tolerated dose level as the MTD.

Declare a dose level the Maximum Administered Dose (MAD) level without observation of a DLT. Thus, the RP2D would not be the MTD.

Recommend testing of an intermediate dose level.

Recommend protocol amendment to increase patient safety.

Discontinue enrollment and/or the study.

Following review by the SSC, dosing of patients may be stopped in the interest of patient safety based on these general safety criteria:

Any serious adverse event (SAE) that is considered potentially life-threatening and is assessed by the Medical Monitor as related to investigational product.

Any other clinically significant change that indicates to the Investigator or Sponsor a major tolerability concern.

### Study Population

The study population includes patients who are HLA-A*02+ with advanced-stage HPV16+ solid tumors (head and neck, cervical cancer, and other tumor types).

Patients may have received prior therapy with a PD-1, CTLA-4 inhibitor or other immune checkpoint inhibitor.

Number of Patients

The number of patients will depend on safety and observed immunogenic effects. In the monotherapy Part 1 (Escalation Phase), it is anticipated that approximately 9 to 36 DLT-evaluable patients could be enrolled. If none of the planned cohorts indicate that the MTD has been reached, additional cell dose levels or regimens may be tested. Up to a total of approximately 36 evaluable patients could be enrolled in Part 2 (n=12 per cohort). Depending on the need to replace patients within cohorts, it is expected that approximately 72 evaluable patients will be treated in the study.

### Inclusion Criteria

1. Male or female patients ≥18 years of age who are HLA-A*02+, as confirmed by genotyping assay from blood.
2. Histologically confirmed incurable or metastatic solid tumors (including but not limited to cervical and head and neck tumors) that are HPV16+.
3. For cervical cancer, which is not amenable to curative treatment with surgery, radiation, and/or chemoradiation therapy, the cancer must have progressed after prior systemic chemotherapeutic treatment with a platinum-based regimen in the adjuvant or recurrent setting. Patients must have progressive disease while receiving or after the completion of the most recent prior treatment.
   For patients who are intolerant to or refuse a platinum-based systemic chemotherapeutic treatment for recurrent disease, reasons must be documented.
4. For recurrent and metastatic head and neck cancer, which is not amenable to curative treatment with surgery, radiation, and/or chemoradiation therapy, the cancer must have progressed following at least 1 prior platinum-based chemotherapy in the primary, adjuvant or recurrent setting and been offered checkpoint immunotherapy. Patients who relapsed after platinum-containing definitive chemoradiation or after adjuvant chemoradiation are eligible if a platinum re-challenge at time of relapse is not seen as beneficial.
   For patients who are intolerant to or refuse a platinum-based systemic chemotherapeutic treatment for recurrent disease, reasons must be documented.
5. Patients with incurable or metastatic HPV16+ cancers other than cervical or head and neck cancer must have progressed after at least 1 available standard therapy for incurable disease, or the patient is intolerant to or refuses standard therapy(ies) or has a tumor for which no standard therapy(ies) exist.

Escalation Phase (Part 1) and Combination Safety Phase (Part 2)
6. Eastern Cooperative Oncology Group (ECOG) performance status (PS) of 0 to 1.
7. Patients must agree to venous access for the blood collection for manufacture of autologous blood product and be willing to have a central line inserted if venous access is an issue.
8. Patients with unresectable or metastatic solid tumors must have a lesion that can be biopsied with acceptable clinical risk and agree to have a fresh biopsy at Screening and on Cycle 2 Day 8 (+2 days).
   a. A lesion in a previously irradiated area could be biopsied as long as there is objective evidence of progression of the lesion before study enrollment.
9. At least 1 measurable lesion according to RECIST 1.1.
   a. A lesion in a previously irradiated area is eligible to be considered as measurable disease if there is objective evidence of progression of the lesion before study enrollment.
10. Adequate organ function and bone marrow reserve as indicated by the following laboratory assessments performed within 14 days prior to the blood collection for manufacture of autologous blood product:
   a. Bone marrow function: absolute neutrophil count ≥1000/μL; hemoglobin ≥9 g/dL; platelet count ≥75,000/μL. NOTE: In stabilized patients with hemoglobin values <9 g/dL, a blood transfusion may be utilized to meet inclusion criterion.
   b. Hepatic function: total serum bilirubin ≤1.5×ULN; serum AST/ALT, ≤2.5×ULN (≤5×ULN in the presence of hepatic metastases); alkaline phosphatase <2.5×ULN with the following exception: patients with liver and bone involvement: alkaline phosphatase ≤5×ULN.
      i. Patients with inherited disorders of bilirubin metabolism should be discussed with the Sponsor.
   c. Renal function: serum creatinine ≤2.5×ULN or creatinine clearance ≥30 mL/min based either on urine collection or Cockcroft-Gault estimation.
   d. Coagulation profile: prothrombin time (PT), international normalized ratio (INR)/partial thromboplastin time (PTT)≤1.5×ULN. Patients on a stable, maintenance regimen of anticoagulant therapy for at least 30 days prior to blood collection for manufacture of autologous blood product may have PT/INR measurements >1.5×ULN if, in the opinion of the Investigator, the patient is suitable for the study. An adequate rationale must be provided to the Sponsor prior to enrollment.
11. Patients with immune-mediated endocrinopathies following treatment with immune checkpoint inhibitors requiring hormone replacement therapy are eligible.
   a. Patients requiring prednisone as part of hormone replacement therapy are eligible if the daily doses do not exceed 10 mg.
12. Female patients of childbearing potential must:
   a. Have a negative serum beta human chorionic gonadotropin (β-hCG) pregnancy test at Screening, and
b. Agree to use highly effective contraception from the time of informed consent until at least 5 months after the last dose of immune checkpoint inhibitor or SQZ-AAC-HPV (CTFG, 2020).
   Examples of highly effective contraception include the following:
      Combined hormonal contraceptives (containing both estrogen and progesterone) associated with inhibition of ovulation; may be oral, intravaginal or transdermal)
      Intra-uterine device (IUD)
      Intra-uterine, hormone-releasing system (IUS)
      Bilateral tubal occlusion
      Vasectomized partner
      Sexual abstinence
13. Male patients who are not vasectomized must be willing to use condoms from the time of informed consent until at least 5 months after the last dose of immune checkpoint inhibitor or SQZ-AAC-HPV.
14. The patient is capable of understanding and complying with the protocol and has signed the required informed consent form (ICF). The appropriate ICF must be signed before relevant study procedures are performed. If applicable, the female partner of a male patient understands and signs the pregnant partner ICF.

### Exclusion Criteria

1. Treatment with anticancer therapy, including investigational therapy, within 2 weeks prior to blood collection for manufacture of autologous blood product. For prior therapies with a half-life longer than 3 days, timing of discontinuation of the therapy should be discussed with the Sponsor.
2. Patients with >Grade 1 adverse events (AEs) (except Grade 2 alopecia) according to NCI CTCAE version 5.0 related to previous treatment with anticancer or investigational therapy that do not resolve (i.e., ≤Grade 1 or better) at least 2 weeks prior to blood collection for manufacture of autologous blood product.
3. History of any Grade 4 irAE from prior immunotherapy (patients with endocrinopathy managed with replacement therapy or asymptomatic elevation of serum amylase or lipase are eligible), any irAE that led to permanent discontinuation of prior immunotherapy, or any Grade 3 irAE that occurred ≤6 months prior to blood collection for manufacture of autologous blood product.
4. Patients treated with non-corticosteroid based immunosuppressive agents within the last 6 months may not be eligible and should be discussed with the Sponsor.
5. Patients with active, known, or suspected autoimmune disease may not be eligible and should be discussed with the Sponsor.
6. Patients who have undergone splenectomy.
7. Patients who have received or who are anticipated to require blood transfusion within 4 weeks prior to the blood draw for autologous investigational product manufacture.
   Note: Patients may receive blood transfusions following the blood draw as clinically indicated.
8. Patients with prior allogeneic bone marrow or solid organ transplantation may not be eligible and should be discussed with the Sponsor.
9. Live virus vaccination within 4 weeks prior to blood collection for manufacture of autologous blood product.
10. Systemic treatment with either corticosteroids (>10 mg of prednisone or the equivalent per day) or other immunosuppressive medications within 14 days prior to blood collection for manufacture of autologous blood product.
   Note: Inhaled, intranasal, intra-articular and topical (including ocular) steroids are allowed. The use of steroid replacement for patients with adrenal insufficiency is allowed. The use of fludrocortisone for mineralocorticoid replacement in patients with adrenal insufficiency is allowed.
11. Has known active central nervous system metastases and/or carcinomatous meningitis. Patients with previously treated brain metastases may participate provided they are stable (without evidence of progression by imaging for at least 4 weeks prior to the first dose of investigational product and any neurologic symptoms have returned to Baseline), have no evidence of new or enlarging brain metastases, and are not using steroids for at least 7 days prior to blood collection for manufacture of autologous blood product. This exception does not include carcinomatous meningitis, which is excluded regardless of clinical status.
12. History of interstitial lung disease requiring steroids, idiopathic pulmonary fibrosis, pneumonitis (including drug induced), or organizing pneumonia (e.g., bronchiolitis obliterans, cryptogenic organizing pneumonia).
   a. Patients with asymptomatic pneumonitis who have not required steroid therapy for pneumonitis are eligible.
13. Clinically significant cardiac disease, including unstable angina, acute myocardial infarction within 6 months prior to blood collection for manufacture of autologous blood product, New York Heart Association class III or IV congestive heart failure, and arrhythmia requiring therapy.
14. Systemic arterial thrombotic or embolic events, such as cerebrovascular accident (including ischemic attacks) within 1 month prior to blood collection for manufacture of autologous blood product.
15. Systemic venous thrombotic events (e.g., deep vein thrombosis) or pulmonary arterial events (e.g., pulmonary embolism) within 1 month prior to blood collection for manufacture of autologous blood product.
   a. Patients with venous thrombotic events before blood collection for manufacture of autologous blood product on stable anticoagulation therapy are eligible.
16. History or presence of an abnormal electrocardiogram (ECG) that, in the Investigator's opinion, is clinically meaningful.
17. Left ventricular ejection fraction (LVEF)<50%.
18. Major surgery within 2 weeks of blood collection for manufacture of autologous blood product; following major surgeries >2 weeks prior to blood collection for manufacture of autologous blood product, all surgical wounds must be healed and free of infection or dehiscence.
19. Any other clinically significant comorbidities, such as active infection, known psychiatric or neurological disorder, or any other condition, which in the judgment of the Investigator, could compromise compliance with the protocol, interfere with the interpretation of study results, or predispose the patient to safety risks.
20. Known active hepatitis B or hepatitis C, or active Mycobacterium tuberculosis infection.
21. Patient has history of alcohol and/or illicit drug abuse within 12 months of entry.
22. Female patients who are breastfeeding or have a positive serum pregnancy test at the Screening visit.
23. Patient has a history of allergy or hypersensitivity to any component of SQZ-AAC-HPV.
24. History of severe allergic anaphylactic reactions to chimeric, human, or humanized antibodies or infusion proteins (combination cohorts only).
25. Known hypersensitivity to ipilimumab, nivolumab, Chinese hamster ovary cell products or any component of the ipilimumab or nivolumab formulation (combination cohorts only).
26. Enrollment of HIV+ patients should be discussed with the Sponsor.

### Blood Collection for Manufacture of Autologous Blood Product.

The goal of the blood collection for autologous product manufacture is to provide a yield of RBCs for each patient of approximately 500×10⁹ cells to support extended treatment duration. To this end, at least 200 mL of whole blood (±10%) is drawn in order to collect at least 500×10⁹ RBCs. In accordance with local procedures, an RBC or complete blood cell count is taken during blood collection so that the processed blood volume may be increased. In the event an RBC or complete blood cell count cannot be taken during blood collection, a sample is taken at the end of blood collection, if possible, to determine the RBC count in the RBC collection. The results should be processed as soon as possible and provided to the Sponsor in real-time.

### Tumor Response Assessment and Schedule

Tumor assessment is performed at Screening (baseline) and tumor response is assessed by the Investigator every 9 weeks (±7 days) for 1 year after the first dose of SQZ AAC HPV, then every 12 weeks (±7 days) thereafter until disease progression as confirmed by RECIST and iRECIST, unacceptable toxicity, withdrawal of consent, death or 2 years from the date of the first administration of SQZ AAC HPV, whichever occurs first.

For patients who achieve a partial response (PR) or complete response (CR), tumor assessment is repeated 4 weeks later to confirm response.

Disease is evaluated via radiographic imaging, either CT scan or MRI; radiographic methods is consistent throughout the study. Patients who experience disease progression per RECIST 1.1 may continue dosing if considered in their best interest by the treating Investigator to allow for confirmation of disease progression; i.e., iCPD according to iRECIST (Seymour et al, 2017).

If a patient discontinues investigational product for reasons other than progression, that patient continues to be imaged following the schedule outlined above. If a patient discontinues treatment due to clinical deterioration, the TEAEs associated with the clinical progression is recorded on the AE page. Radiographic assessments should be obtained and recorded.

At Screening and all subsequent time points, cervical, anal/rectal, vulvar/vaginal, and penile carcinomas require computed tomography (CT) of the torso (chest, abdomen and pelvis) and all known sites of disease; oropharyngeal carcinomas require CT of head, neck, and chest and other areas of known involvement. If, for justifiable reasons, CT scans cannot be used or do not allow for an appropriate tumor assessment, magnetic resonance imaging (MRI) is permitted and the Sponsor is informed during Screening. The same radiographic procedure used to assess disease sites at Screening is used throughout the study. For all other advanced solid tumor types, the Investigator images all known sites of disease using the imaging modality the Investigator believes best for that tumor type.

Magnetic resonance imaging of the brain is required at Screening in all patients with a history of brain metastases, and may be repeated at subsequent time points in any patient with a history of brain metastases and/or in any patient who develops symptoms suggestive of brain metastasis. If a patient is unable to tolerate or has a contraindication for MRI, CT scan is used.

If possible, the same evaluator performs assessments to ensure internal consistency across visits. At the Investigator's discretion, CT scans are repeated at any time if progressive disease (PD) is suspected. For patients who achieve a partial response (PR) or complete response (CR), tumor assessment is repeated 4 weeks later to confirm response.

### Pharmacodynamic Assessments, Including Immunogenic Measurements

### Sample Collection Schedule

For assessing the effect of SQZ-AAC-HPV on pharmacodynamics including immunogenic measurements, blood and tumor biopsy samples are collected.

### Tumor Biopsies

Prior to blood collection for manufacture of autologous blood product, patients undergo a Screening tumor biopsy (primary tumor or metastasis) that can be from a previously radiated site with active tumor growth. All patients are required to undergo a repeat tumor biopsy of the same primary tumor or metastasis on Cycle 2 Day 8 (±2 days). If possible, an additional repeat tumor biopsy is obtained (predose) at Cycle 5 Day 1 (±2 days); this sample is optional. If preliminary data suggest that modification of the on treatment tumor biopsy time point would be more appropriate, alternative on-treatment tumor biopsy time points may be considered.

Tumor tissue should be of good quality based on total and viable tumor content. The fresh tumor biopsy taken at Screening from the primary tumor or metastasis site and subsequent biopsies are from the same primary tumor or metastasis biopsied at Screening. The anatomical location (organ and region within organ) should be noted on the CRF.

### Pharmacodynamic Assessments

Whenever possible, baseline samples are used for longitudinal assessment of cellular correlative tests, including, but not limited to, immunophenotyping by flow cytometry including tetramer staining, assessment of T cell production of cytokines following co-culture with HPV peptides (IFNγ and Granzyme B enzyme-linked immunoSpot [ELISPOT]), and circulating cell free HPV16 DNA (cfHPV DNA). Baseline tumor biopsies and selected blood samples will be used for comparison to post-treatment samples only (Table 2).

TABLE 2
Pharmacodynamic and Immunogenic Assessments

| Sample Source | Assay |
|---|---|
| Blood | IFNγ and GranzymeB ELISPOT (with and without in vitro stimulation) |
| | E6, E7 tetramer staining (combine with surface marker staining) |
| | Circulating cell-free HPV16 DNA (cfHPV DNA) levels |
| Tumor tissue | Immunohistochemistly assessment of changes in tumor micro-environment |

Abbreviations:
DNA = deoxyribonucleic acid;
HPV16 = human papilloma virus strain 16; [symbol]
= interferon gamma

Evaluation for development of endogenous immune responses via ELISPOT, T cell receptor sequencing, and epitope spreading may be conducted. The information about endogenous immune responses detected via ELISPOT will inform the immunohistochemical analysis of tumor biopsies.

### Cytokine Assessments

Patients with Grade 2, 3, or 4 CRS have additional cytokine plasma levels performed during Grade 2, 3, or 4 CRS events. Blood collections are obtained predose, on Cycle 1, Day 1, and at time of diagnosis of a CRS, at time of an increase in severity (e.g., when a Grade 2 CRS progresses to a Grade 3 CRS), onset of neurological symptoms, and at time of discharge or resolution.

The evaluation of a cytokine panel includes, but is not limited to, IFN gamma (IFNγ) and IL 6. Although CRS may have a delayed onset, it rarely presents beyond 14 days after initiation of therapy. Patients exhibiting symptoms consistent with CRS presenting outside this window are carefully evaluated for other causes.

Cytokines are also be monitored for pharmacodynamic assessments. Baseline and post treatment serum samples are collected to assess anti-tumor immune responses by measuring cytokines that could provide information about drug inflammatory responses.

For assessing the kinetics of SQZ-AAC-HPV removal from the blood stream following its IV administration, blood samples are collected in Cycles 1 and 2.

### Safety Assessments

Eligibility criteria for this study have been established to ensure the safety of participating patients. Safety is evaluated in this study through the monitoring of all SAEs and nonserious AEs and laboratory abnormalities, defined and graded according to NCI CTCAE version 5.0. General safety assessments will include physical examinations and specific laboratory evaluations, including serum chemistry, coagulation, and blood counts including differential. SAEs and ≥Grade 2 AESIs will be reported in an expedited fashion for entry into the safety database.

During the conduct of the study, the totality of safety events observed is reviewed (including CRS events that resolved to Grade 2) and a decision will be made if a given event requires initiation of staggered enrollment of patients following this event. Staggered enrollment in potential additional monotherapy cohorts (Part 1) or in the Combination Safety Cohorts (Part 2) requires all subsequent newly enrolled patients in a cohort or cohorts to be staggered by 1 week. Parallel enrollment of patients may continue in some cohorts, if applicable. Patients with Grade 2, 3, or 4 CRS will have additional blood samples taken for safety laboratories and the evaluation of the cytokine panel.

Exposure to immune checkpoint inhibitors may increase the risk of irAEs, specifically autoimmune conditions. As such, irAEs are recognized early and treated promptly to avoid potential major complications.

All patients return to the clinic for a Safety Follow-up visit within 15 to 45 days after the last dose of investigational product. All AEs and SAEs are recorded until 6 weeks after last dose of investigational product (EOD6W) or up to 45 days from drop out or until initiation of another anticancer therapy, whichever occurs first. Only ongoing SAEs determined by the Investigator to be possibly, probably, or definitely related to SQZ-AAC-HPV monotherapy or combination therapy will be followed up.

### Physical Examination and Height and Weight

A physical examination will include height (Screening only), weight, and an assessment of general appearance and an evaluation of the following systems: dermatologic, head, eyes, ears, nose, mouth/throat/neck, thyroid, lymph nodes, respiratory, cardiovascular, gastrointestinal, extremities, musculoskeletal, neurologic, and gynecologic and genitourinary systems, as indicated. It is especially important to capture weight during the physical examination of the patient within 24 hours of blood collection for manufacture of autologous blood product, as patient dosing is determined by weight.

### Performance Status

Eastern Cooperative Oncology Group scales and criteria are used to assess a patient's performance status, assess how the disease affects the daily living abilities of the patient, and determine appropriate treatment and prognosis.

### 12-Lead Electrocardiograms

12-lead ECGs are performed by qualified site personnel at scheduled time points using an ECG machine that determines heart rate, PR interval, QRS interval, RR interval, and QT interval. QTcB (QTc corrected by Bazett's formula) and/or QTcF (QTc corrected by Fridericia's formula) will be calculated based on the QT and RR intervals. During the collection of ECGs, patients should be in a resting position, in a quiet setting without distractions (e.g., without television, cell phones) for at least 10 minutes before ECG collection.

All ECGs are evaluated by a qualified physician for the presence of abnormalities. Echocardiograms

Echocardiogram or multigated acquisition (MUGA) scans will be performed to measure LVEF at Screening and as clinically indicated.

### Laboratory Assessments

Samples for clinical laboratory assessments are collected at time points. Clinical laboratory tests outlined in Table 3 are performed by the site. Samples for laboratory tests outlined in Table 3 will be collected in appropriate tubes and handled according to standard procedures of the site.

Clinical laboratory variables are listed in Table 3. For safety monitoring purposes, the Investigator must review, sign, and date all out of range laboratory results. Laboratory results must be documented.

TABLE 3
Clinical Laboratory Assessments
Hematology^{a}: Required at all visits.

| | |
|---|---|
| Total white blood cell count | Neutrophils (percentage and absolute count) |
| Red blood cell count | Lymphocytes (percentage and absolute count) |
| Hemoglobin | Monocytes (percentage and absolute count) |
| Hematocrit | Eosinophils (percentage and absolute count) |
| Mean corpuscular volume | Basophils (percentage and absolute count) |
| Mean corpuscular hemoglobin | Platelet count |
| Mean corpuscular hemoglobin | Red blood cell distribution width |
| concentration | |

Coagulation^{a,b,} Required at all visits unless otherwise specified.

| | |
|---|---|
| Prothrombin time (PT) | International normalized ratio (INR) |
| Partial thromboplastin time (PTT) | D-dimer |
| Fibrinogen | von Willebrand factor |

Clinical Chemistry: Required at all visits except blood collection
for manufacture of autologous blood product.

| | |
|---|---|
| Alanine aminotransferase (ALT) | Gamma glutamyl transferase |
| Albumin | Glucose |
| Alkaline phosphatase | Lactate dehydrogenase |
| Aspartate aminotransferase (AST) | Phosphorus |
| Blood urea nitrogen | Potassium |
| Calcium | Sodium |
| Chloride | Thyroid function test (TSH, |
| | free T3, and free T4) |
| Cholesterol | Total bilimbin |
| Creatinine | Total protein |
| C-reactive protein | Triglycerides |
| Creatine kinase | Uric acid |
| Ferritin | Magnesium |

Urinalysis: Required at all visits except blood collection for
manufacture of autologous blood product.

| | |
|---|---|
| Bilirubin | Blood |
| Glucose | pH and specific gravity |
| Ketones | Protein |
| Leukocytes | Urobilinogen |
| Nitrite | Leukocyte esterase |

Microscopic (if macroscopic panel is abnormal) white blood cells, RBC,
casts, crystals, bacteria, and epithelial cells
Viral Serology: Required at Screening and as clinically indicated.

| | |
|---|---|
| Hepatitis B core antibody (anti-HBc) | Human immunodeficiency virus (HIV) |
| IgM antibody to anti-HBc | (Types 1 and 2) antibodies |
| (IgM anti-HBc) | |
| Hepatitis B surface antigen (HBsAg) | Hepatitis C vims antibody (anti-HCV) |

Pregnancy Testing: Required at Screening, on day 1 of each cycle and 6 weeks
after the last administration of investigational product.
Semm human beta chorionic gonadotrophin (women of childbearing potential only)
required at screening; urine β-hCG may be used in subsequent assessments.
^{a}Results for these laboratory tests are required to be collected prior to, or the day of, blood collection for manufacture of autologous blood product, with the results available prior to blood collection for manufacture of autologous blood product.
^{b}Results of coagulation parameters are required on the day of, or the day following, any tumor biopsy.
Abbreviations:
CRS = cytokine-release syndrome;
T3 = triiodothyronine;
T4 = thyroxine;
TSH = thyroid-stimulating hormone
ADVERSE EVENT

### Adverse Event

An AE is any untoward medical occurrence in a patient that does not necessarily have a causal relationship with the investigational product administered. An AE can therefore be any unfavorable or unintended sign (including an abnormal laboratory finding), symptom, or disease temporally associated with the use of an investigational product, whether or not related to the investigational product. Adverse events may be new events or may be pre-existing conditions that have become aggravated or have worsened in severity or frequency.

Adverse events may be clinically significant changes from Baseline in physical examination, laboratory tests, or other diagnostic investigation.

In this study, an AE is treatment-emergent if the onset time is after administration of investigational product through 6 weeks after the last dose of study treatment (SQZ-AAC-HPV or the immune checkpoint inhibitor).

### Serious Adverse Event

An SAE is any AE that results in any of the following:
Death.
Is immediately life-threatening.
Requires in-patient hospitalization or prolongation of existing hospitalization.
Results in persistent or significant disability or incapacity.
Results in a congenital abnormality or birth defect.
Is an important medical event that may jeopardize the patient or may require medical intervention to prevent 1 of the outcomes listed above.

All SAEs that occur after any patient has signed the ICF, before treatment, during treatment, or within 30 days following the cessation of treatment, whether or not they are related to the study, must be recorded on the appropriate clinical procedure form.

### Adverse Events of Special Interest

An AESI is an AE (serious or nonserious) of scientific and medical concern specific to investigational product, for which ongoing monitoring and immediate notification by the Investigator to the Sponsor is required. Such AEs may require further investigation to characterize and understand them. Adverse events of special interest may be added or removed during the study by a protocol amendment.

The following AEs are considered AESIs:
Events suggestive of hypersensitivity, cytokine release, systemic inflammatory response syndrome, systemic inflammatory activation.
Influenza-like illness.
Infusion-reaction syndrome.
irAEs related to immune therapy, such as myocarditis, neurological irAEs, transaminitis of immune-related etiology, and nephritis.
   All AESIs of Grade 2 or higher will be reported to the Sponsor within 24 hours of awareness. Events that are considered by the Investigator to be irAEs or suspected to be immune-related should be discussed with the Sponsor immediately.

In addition, the following events will be reported to the Sponsor:
A suspected overdose of SQZ-AAC-HPV.
Liver tests abnormalities meeting Hy's law criteria, i.e., an AST or ALT laboratory value ≥3×ULN and a total bilirubin laboratory value ≥2×ULN and, at the same time, an alkaline phosphatase laboratory value <2×ULN, as determined by protocol specified or unscheduled laboratory testing.

### General

Adverse events, including SAEs, are collected for each patient from the date the first ICF is signed until EOD6W or up to 45 days from drop out or until initiation of another anticancer therapy, whichever occurs first. All SAEs and ≥Grade 2 AESIs that occur within the reporting period, regardless of causality, must be reported by the Investigator to the Sponsor or designee within 24 hours from the time the Investigator becomes aware of the SAE or AESI. Only ongoing SAEs determined by the Investigator to be possibly, probably, or definitely related to SQZ AAC HPV monotherapy or combination therapy will be followed up.

The AE term is reported in standard medical terminology when possible. For each AE, the Investigator will evaluate and report the onset (date and time), resolution (date and time), severity, causality, action taken, whether serious, and whether or not it caused the patient to discontinue the study or resulted in a modification or delay of investigational product administration.

All AEs (both expected and unexpected), spontaneously reported by the patient or in response to an open question from the study personnel or revealed by observation, will be documented on the appropriate study-specific clinical procedure forms during the study at the study site. Clinical outcomes or symptoms related to PD are reported as SAEs and/or deaths if they meet SAE and/or death criteria and occur within 6 weeks of the last investigational product administration. They are reported according to the diagnosis or symptom of event and not by the term “progressive disease.”

Any laboratory value outside the normal range are flagged for the attention of the Investigator or designee at the site. The Investigator or designee will review for clinical significance. If a clinically significant abnormality is found in the samples taken after dosing, during the study, and/or within 6 weeks following the cessation of investigational product, it should be recorded as an AE and the patient followed until the test(s) has (have) normalized or stabilized, at the discretion of the Investigator. Abnormal laboratory values that constitute an SAE or lead to discontinuation of administration of investigational product are reported and recorded on the AE page of the case report form (CRF).

SAEs and AESIs are followed until resolution, the condition stabilizes, or the Investigator and Sponsor agree that follow up is not required. If the event has not resolved at the end of the study reporting period, it must be documented as ongoing. All SAEs and nonserious ≥Grade 2 AESIs are reported to the Global Pharmacovigilance Processing Group within 24 hours of learning of the event.

### Assessment of Severity

The NCI CTCAE version 5.0 are used to assess and grade severity for AEs and for laboratory abnormalities. ASTCT Consensus Grading is used for CRS and ICANS. Each AE term are mapped to the latest version of Medical Dictionary for Regulatory Activities (MedDRA) term and code.

If the event is not covered in CTCAE version 5.0, the guidelines shown in Table 4 are to assess severity.

TABLE 4
Severity and Toxicity Grade of Events Not Covered by CTCAE

| Toxicity | | |
|---|---|---|
| Grade | Severity | Description |
| Grade 1 | Mild | Transient or mild discomfort (<48 hours); no medical |
| | | intervention/therapy required. |
| Grade 2 | Moderate | Mild to moderate limitation in activity-some assistance may be |
| | | needed; no or minimal medical intervention/therapy required. |
| Grade 3 | Severe | Marked limitation in activity, some assistance usually required; |
| | | medical intervention/therapy required, hospitalization possible. |
| Grade 4 | Life-threatening | Extreme limitation in activity, significant assistance required; |
| | | significant medical intervention/therapy required, hospitalization |
| | | or hospice care possible. |
| Grade 5 | Fatal | The patient died due to the event. |

Source: (NIAID, 2003)
Abbreviations CTCAE = Common Terminology Criteria for Adverse Events

### Assessment of Causality

Relationship to investigational product are assessed by the Investigator. Accordingly, the AE and SAE report forms include the option to attribute causality to SQZ-AAC-HPV, ipilimumab, nivolumab, or a combination. For patients receiving combination therapy with SQZ AAC-HPV and immune checkpoint inhibitor(s), causality is assessed individually for each protocol-specified therapy. A reasonable suspected causal relationship is attributed to the immune checkpoint inhibitor alone if the event is consistent with the immune checkpoint inhibitor labeling.

The relationship of the AE to investigational product (i.e., SQZ-AAC-HPV, ipilimumab, nivolumab, or a combination) is documented as follows: Definite: The AE is clearly related to the investigational product.
Probable: The AE is likely related to the investigational product.
Possible: The AE may be related to the investigational product.
Unlikely: The AE is doubtfully related to the investigational product.
Unrelated: The AE is clearly NOT related to the investigational product.

An Investigator who is qualified in medicine makes the determination of the relationship to the investigational product for each AE. The Investigator decides whether, in his or her medical judgment, there is a reasonable possibility that the event may have been caused by the investigational product. If no valid reason exists for suggesting a relationship, then the AE is classified as “unrelated.” If there is any valid reason, even if undetermined, for suspecting a possible cause-and-effect relationship between the investigational product and the occurrence of the AE, then the AE will be considered “related.”

If the relationship between the AE/SAE and the investigational product is determined to be “definite,” “probable”, or “possible” the event is considered related to the investigational product for the purposes of expedited regulatory reporting.

### Expectedness

An AE that is not listed in, or is inconsistent with the specificity or severity, from the applicable product information (e.g., the IB for SQZ-AAC-HPV or the approved labeling for ipilimumab or nivolumab) is considered unexpected.

### Efficacy Analyses

### Definitions

Progression-free Survival (PFS) is defined as the time from Cycle 1 Day 1 to first documentation of objective tumor progression (PD, radiological) according to RECIST 1.1 or death due to any cause, whichever comes first. Progression-free survival data will be censored on the date of last tumor assessment documenting absence of PD for patients who do not have objective tumor progression and are still on study at the time of the analysis, are given antitumor treatment other than investigational product, or are removed from treatment follow-up prior to documentation of objective tumor progression. Patients having no tumor assessments after enrollment who are not known to have died will have PFS censored on Cycle 1 Day 1. PFS is assessed by both RECIST 1.1 and iRECIST criteria to accommodate different practice across participating sites.

Overall Survival (OS) is defined as the time from the date of Cycle 1 Day 1 to date of death due to any cause. In the absence of confirmation of death, survival time is censored at the last date the patient is known to be alive. Patients lacking data beyond Cycle 1 Day 1 will have their survival times censored on Cycle 1 Day 1.

Objective Response Rate (ORR) is defined as the proportion of patients with CR or PR according to RECIST 1.1. Objective response rate is provided as unconfirmed and confirmed ORR. Confirmed responses are those that persist on repeat imaging study at least 28 days after the initial documentation of response. Similarly, iORR by iRECIST will also be summarized and reported.

Duration of Response (DoR) is defined as the time from the first documentation of PR or CR to the first documentation of objective tumor progression or death due to any cause. Duration of response data is censored on the day of the last tumor assessment documenting absence of PD for patients who: 1) do not have tumor progression and are still on the study at the time of an analysis; 2) are given antitumor treatment other than the investigational product; or 3) are removed from the study follow-up prior to documentation of objective tumor progression. Similarly, iDoR by iRECIST is summarized and reported.

Best Overall Response (BOR) is determined once all tumor assessments from Cycle 1 Day 1 until disease progression or death are recorded. In general, it is the best response across all assessments; however, confirmation of CR, PR, and stable disease (SD) is used in BOR determination. To confirm CR or PR, changes in tumor measurements is confirmed by repeat assessments that should be no less than 4 weeks (28 days) after the criteria for response are first met. To confirm SD, it must have occurred at least 12 weeks from Cycle 1 Day 1; otherwise, BOR will depend on subsequent assessments. Best overall response will be summarized by percentages and as a time to event variable for time to best response using enrollment as the anchor date. Similarly, iBOR by iRECIST is summarized and reported.

Disease Control Rate (DCR) is the proportion of patients in whom the BOR is determined as CR, PR, or SD by RECIST 1.1 at defined time points. All patients in the safety population with measurable disease at Baseline and eligible for tumor assessment is considered as the denominator of the DCR proportion at 3, 6, and 12 months. Similarly, iDCR by iRECIST is summarized and reported.

### Analyses

Efficacy analyses is performed on the safety population. Antitumor activity (ORR, PFS, OS) will be described for patients with documented HLA class I expression as well. If the Per Protocol population differs from the Safety Population, efficacy analyses will be also performed using the PP population.

All assessments using response assessments by RECIST 1.1 or iRECIST is analyzed using the Investigators' review assessments.

The Kaplan-Meier method is used to estimate the median PFS and 2-sided 95% confidence interval. Patients who die, regardless of cause of death, will be considered to have had an event unless subsequent anticancer therapy was received prior to death. If subsequent therapy is received, the patient will be censored of date of last evaluable tumor assessment prior to subsequent therapy. Patients who withdraw consent for the study are considered censored at the time of the last evaluable tumor assessment prior to withdrawing consent. Patients who are still alive at the time of the clinical data cut-off date will be censored at the most recent evaluable tumor assessment. All patients who were lost to follow-up prior to the clinical data cut-off date will also be considered censored at the time of the last evaluable tumor assessment prior to lost to follow up.

Duration of response, time to best overall response, and overall survival will use the same censoring algorithm as PFS. In addition, iPFS, iBOR, iDCR, and time to iBOR using iRECIST are analyzed and reported using similar methods.

Objective Response Rate (ORR) and DCR are presented as a proportion with a 95% 2-sided confidence interval based on the exact binomial distribution. SD lasting at least 12 weeks will be reported as point estimates.

### Safety Analyses

All safety parameters are analyzed using the Safety population. Safety parameters include AEs, laboratory evaluations, vital signs, ECOG, exposure, ECG, ECHO/MUGA and physical examinations.

The primary endpoint for safety is the number of patients with any AE and observed toxicity to SQZ-AAC-HPV administration, where the severity is assessed using NCI CTCAE version 5.0. All AEs with onset after the first administration of SQZ-AAC-HPV will be included in the analysis. Adverse events are collected beginning at signing informed consent; however, analyses will be performed focusing on treatment-emergent AEs.

The AEs will be analyzed using descriptive statistics. For patients with multiple incidences of a given AE, the highest severity is used.

### Adverse Events

The AEs are coded using the current version of the MedDRA coding dictionary.

An AE is treatment-emergent if the onset occurs on Cycle 1 Day 1 through 6 weeks after the last dose of investigational product. For AEs with partial onset times, non-missing date parts are used to determine if the AE is treatment-emergent. If a determination cannot be made as to when the AE occurred relative to investigational product administration, the AE will be classified as treatment-emergent. Treatment-emergent AEs also include any AEs that were present prior to the first administration of investigational product and worsened in toxicity after the administration.

The analyses described in this section are based on TEAEs, plainly referred to as AEs in this section for brevity.

Adverse events considered as possibly, probably, or definitely related to investigational product by the Investigator are classified as related for summary purposes.

The number and percentage of patients with any AE, any related AE, any SAE, any related SAE, any Grade 3 or higher AE, any related Grade 3 or higher AE, as well as the total number of events for each category, are summarized. The number of deaths due to an AE, hospitalization due to an AE, and treatment discontinuation due to an AE, as well as DLTs and AESIs, are summarized.

The number and percentage of patients with an AE, as well as the total number of AEs, are summarized by system organ class and preferred term. This tabulation will be repeated for related AEs, AESIs, SAEs, related SAEs, and ≥Grade 3 AEs, and related ≥Grade 3 AEs.

All AEs, including non-TEAEs, are provided in patient listings. Patient listings of AEs causing discontinuation of investigational product, AEs leading to death, SAEs, related AEs, AESI, DLTs, and ≥Grade 3 AEs will be produced.

### Clinical Laboratory Evaluation

Baseline is defined as the last non-missing value prior to the first exposure to investigational product. This is typically Cycle Day 1 pre-dose, but may be earlier. Actual values and changes from Baseline clinical laboratory tests are summarized by study visit.

Laboratory test results are classified according to NCI CTCAE version 5.0 and clinical significance as determined by the Investigator. If more than 1 laboratory result is reported per study visit per parameter, the result yielding the most severe classification will be selected for analysis. Shift tables are created to show the greatest change from baseline for graded laboratory parameters.

All laboratory assessments are provided in listings.

Patients with clinically significant abnormal laboratory test results are listed. This listing will include all results of the laboratory parameter that was abnormal and determined to be clinically significant by the Investigator for a patient across study visit.

### Vital Signs

Baseline is defined as the last non missing value prior to the first exposure to investigational product. Actual values and changes from Baseline in vital signs will be summarized by study visit and study time point. All vital sign data are presented in patient listings.

Vital sign values are classified according to the clinical significance as determined by the Investigator. The number of patients with a non-missing result, the number and percentage of patients with a non-clinically significant result, and clinically significant result will be summarized by study visit and study time point. If more than 1 vital sign result is reported per study visit and study time point per parameter, the result yielding the most severe classification will be selected for analysis.

Patients with clinically significant vital sign values are listed. This listing includes all results of the vital sign parameter that was determined by the Investigator to be clinically significant for a patient across study time points.

### Physical Examination

Abnormal physical examination findings are listed.

### 12-Lead ECG

ECG results is presented in a shift table (normal, abnormal not clinically significant, abnormal, clinically significant) to show the greatest change from baseline. All ECG results are presented in patient listings.

### Other Safety Variables

All safety data will be provided in listings.

ECOG PS and change from Baseline in ECOG PS are summarized at each scheduled visit that it is collected. Change from Baseline in ECOG PS are summarized as a continuous variable and as a categorical variable. A decrease of ≥1 point from Baseline are categorized as an “improvement” from Baseline. An increase of ≥1 point from Baseline are categorized as a “deterioration” from Baseline. Improvement, deterioration, and unchanged ECOG PS from Baseline is summarized as a categorical variable by treatment at each post-enrollment time point that ECOG PS is evaluated.

### Pharmacodynamic Analyses

Biomarkers are summarized for each time point, for change from Baseline and % change from Baseline. Correlation between pharmacodynamic markers and SQZ-AAC-HPV are explored with descriptive and graphical methods.

Descriptive statistics (mean, standard deviation, median, minimum, maximum, and geometric mean) for each marker are reported. Graphs of individual values over time according to dose group will be presented.

### Dose Manufacturing Feasibility

Dose manufacturing feasibility is assessed based on individual patient batch yield, product failures prohibiting use, and any additional information from blood collection for manufacture of autologous blood product through SQZ AAC HPV product production that is deemed relevant.

### Example 2. Production of M-AAC-HPV and Characterization of Surface Phosphatidylserine Based on Annexin V+ Cells as Measured by Flow Cytometry

The objectives of these studies was to characterize surface phosphatidylserine (PS) levels via annexin V staining of M-AAC-HPV and flow cytometry analysis.

### Description of the Processes Used to Generate M-AAC-HPV

To generate M-AAC-HPV, mouse RBCs are SQZ processed with E7 synthetic long peptide (SLP) and the adjuvant polyinosinic-polycytidylic acid (poly I:C). The mouse E7 SLP, shown below in bold and underlined, includes the mouse E7 antigenic epitope presented on the C57BL/6J class I MHC H2-Kb. This sequence is contained within the same HPV16 E7 protein from which the human E7 SLP is derived. It is noted that for C57BL/6J mice, E7 is the immunodominant antigen and that immunization against E6 provides little therapeutic benefit in the HPV16 TC-1 tumor model (Oosterhuis 2011; Peng 2016, Li 2010). Hence, M-AAC-HPV contains only the mouse E7 SLP. Below is a comparison of the structures of mouse E7 SLP and human E7 SLP.

### MOUSE E7 SLP: GQAEPDRAHYNIVTFSSKSDSTLRLSVQSTHVDIR (SEQ ID NO:25)

HUMAN E7 SLP: QLCTELQTYMLDLQPETTYCKQQLL (SEQ ID NO:23)

The adjuvant used in the production of M-AAC-HPV is the same adjuvant, poly I:C, that is used in SQZ-AAC-HPV, the human drug product.

In addition to facilitating the delivery of E7 SLP and poly I:C to the interior of M-AAC-HPV, the SQZ process increases the levels of exposed PS on the M-AAC-HPV membranes. This surface PS is hypothesized to serve as the ligand recognized by receptors on antigen presenting cells that internalize the M-AAC-HPV after intravenous administration. A fluorescent derivative of annexin V, a phospholipid-binding protein that binds to PS with high affinity (Koopman 1994), is used to stain cells for cell surface PS and is detected using flow cytometry.

Whole blood was harvested from mice, and the mouse RBCs were isolated. The mouse RBCs were then suspended at 1×10⁹ cells/mL in a solution containing the antigen (mouse E7 SLP; 100 M) and the adjuvant (poly I:C; 1 mg/mL) in either PBS (phosphate buffered saline) or RPMI (Roswell Park Memorial Institute (culture medium)), because these studies compared the use of PBS or RPMI in this process. The resulting cell suspension was transferred to the syringe of the small-scale SQZ equipment, and then subjected to SQZ processing. Following SQZ processing, suspensions of the resultant AACs were incubated at room temperature for 20-60 minutes. The M-AAC-HPV suspension was then washed with PBS using centrifugation, and ultimately resuspended to 2×10⁹/mL with PBS.

### Surface PS Levels on M-AAC-HPV

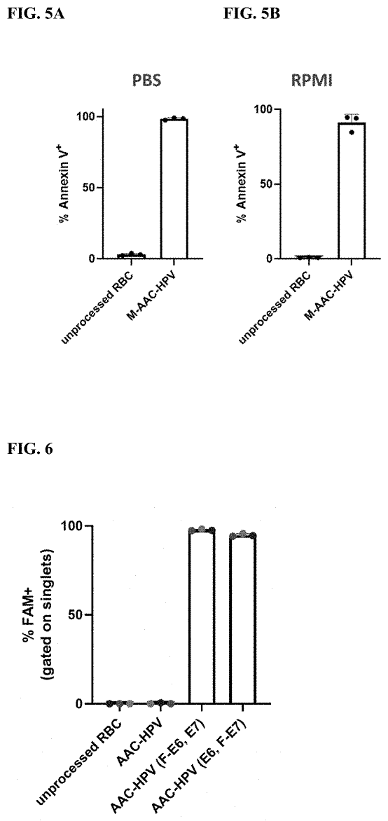

Surface PS levels were characterized via annexin V staining and analysis of data acquired by flow cytometry. Summary data displaying the percentage of M-AAC-HPV that is annexin V+ are shown in FIG. 5A and FIG. 5B).

The average percentage of annexin V+M-AAC-HPV from 6 independently prepared batches (3 batches SQZ processed in PBS and 3 batches SQZ processed in RPMI) was 94.8±5.3% (mean±standard deviation) and the average percent of annexin V+ unprocessed RBCs was 2.0±1.3%. The average ratio of annexin V MFI (geometric mean fluorescence intensity) in M-AAC-HPV to that in unprocessed RBCs from 6 independently prepared batches was 99±59 (mean±standard deviation). In addition, the ratio of annexin V MFI of M-AAC-HPV to the annexin V MFI in unprocessed RBCs was not significantly different when cells were processed in PBS or RPMI.

These studies demonstrate that surface levels of PS are elevated under the conditions used for SQZ processing of RBCs. The percent of annexin V+ AACs for M-AAC-HPV is comparable to that of the human product SQZ-AAC-HPV, which is at least 95.8%.

### Example 3. Cellular Characterization of AAC-HPV by Flow Cytometry

The objectives of the studies to quantify delivery of the FAM (5-carboxy-fluorescein) labeled SLPs (synthetic long peptides), FAM-E6 and FAM-E7 SLPs to AACs, and to characterize surface phosphatidylserine (PS) levels on AACs.

### Description of the Small-Scale Process Used to Generate Human AACs

Whole blood was collected the day before the studies, and the RBCs were isolated the day of the studies. The RBCs were then suspended at 2×10⁹ cells/mL in a solution containing antigens (SLPs) and the adjuvant (poly I:C). The resulting cell suspension was incubated on ice for 10 minutes, transferred to the syringe of the small-scale SQZ equipment, and then subjected to SQZ processing. Following SQZ processing, suspensions of the resultant AACs were incubated at 2-8° C. for 20 minutes and then at 37° C. for 60 minutes. The AAC suspensions were then washed with PBS using centrifugation, and ultimately resuspended to 2×10⁹/mL with PBS.

### Delivery of the FAM-E6 SLP and FAM-E7 SLP to AACs

RBCs isolated from three healthy donors in three separate experiments were each: A) used as is (not SQZ processed) as a control, B) SQZ processed with unlabeled E6 SLP, unlabeled E7 SLP, and poly I:C to generate AAC-HPV, C) SQZ processed with 5-carboxy-fluorescein (FAM)-labeled E6 SLP, unlabeled E7 SLP, and poly I:C to generate AAC-HPV (F-E6, E7), or D) SQZ processed with FAM-labeled E7 SLP, unlabeled E6 SLP, and poly I:C to generate AAC-HPV (F-E7, E6). Table 3 describes these experimental groups. Three batches of human RBCs, each batch from a different donor, were SQZ-processed to generate AAC-HPV, AAC-HPV (F-E6, E7) and AAC-HPV (E6, F-E7). The SQZ processed cells were stained with AF647-annexin V and analyzed by flow cytometry to quantify incorporation of fluorescently labeled SLPs and assess surface PS levels (based on annexin V; results described in 2.4.3). Unprocessed RBCs and AAC-HPV served as negative controls (no FAM label).

TABLE 5
Experimental Groups
SQZ Materials

| Groups | Condition | E6 | E7 | Poly I:C |
|---|---|---|---|---|
| A | unprocessed RBC | NA | NA | NA |
| B | AAC-HPV | 50 pME6 SLP | 200 pME7 SLP | 1 mg/mL |
| C | AAC-HPV (F-E6, E7) | 50 pM FAM-labeled E6 SLP | 200 pME7 SLP | 1 mg/mL |
| D | AAC-HPV (E6, F-E7) | 50 pM E6 SLP | 200 pM FAM-labeled E7 SLP | 1 mg/mL |

NA denotes ‘not applicable’.

Summary data displaying the percentages of FAM-E6 SLP+ and FAM-E7 SLP+ samples are shown in FIG. 6. FAM fluorescence was detected in 0.1% and 0.0% of AAC-HPV and unprocessed RBCs, respectively (the negative controls). The majority of AACs were positive for FAM-E6 SLP or FAM-E7 SLP; an average of 97.8% of AAC-HPV (F-E6, E7) and 95.0% of AAC-HPV (E6, F-E7) were positive for FAM-E6 SLP and FAM-E7 SLP, respectively.

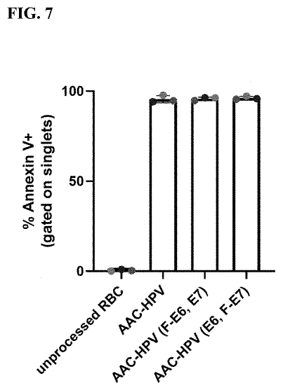

The unprocessed RBCs and the SQZ processed cells were stained with AF647-annexin V and analyzed by flow cytometry to quantify surface PS levels (based on annexin V). Summary data displaying the percentage of annexin V+ samples are shown in FIG. 7. While the average percentage of unprocessed RBCs that were annexin V+ was 1.0%, at least 95.8% of AAC-HPV, AAC-HPV (F-E6, E7) or AAC-HPV (E6, F-E7) were positive for annexin V, demonstrating that the SQZ process increases PS on the plasma membrane.

### Example 4. Imaging of Human RBCs SQZ Processed with Fluorescently-Labeled SLPs

This study demonstrates the intracellular delivery of fluorescently labeled HPV E6 and E7 SLPs into AACs following SQZ processing.

Table 6 describes the experimental groups used in these studies. In each of three independent experiments, RBCs from a healthy donor were isolated from whole blood and SQZ processed with A) 5-carboxy-fluorescein (FAM)-labeled E6 SLP, unlabeled E7 SLP, and poly I:C to generate AAC-HPV (F-E6, E7), B) with FAM-labeled E7 SLP, unlabeled E6 SLP, and poly I:C to generate AAC-HPV (E6, F-E7) or C) with unlabeled E6 SLP, unlabeled E7 SLP, and poly I:C to generate AAC-HPV. The SQZ processed samples were stained with Pacific blue (PB)-conjugated anti-CD235a antibody and imaged with epi fluorescence microscopy. Images for each sample were subjected to line scan analysis to determine whether FAM-labeled SLPs localization was luminal (in the interior of the AAC).

TABLE 6
Experimental Groups
SQZ Materials

| Groups | Condition | E6 | E7 | Poly I:C |
|---|---|---|---|---|
| A | AAC-HPV (F-E6, E7) | 50 pM FAM-labeled E6 SLP | 200 pME7 SLP | 1 mg/mL |
| B | AAC-HPV (E6, F-E7) | 50 pM E6 SLP | 200 pM FAM-labeled E7 SLP | 1 mg/mL |
| C | AAC-HPV | 50 pM E6 SLP | 200 pME7 SLP | 1 mg/mL |

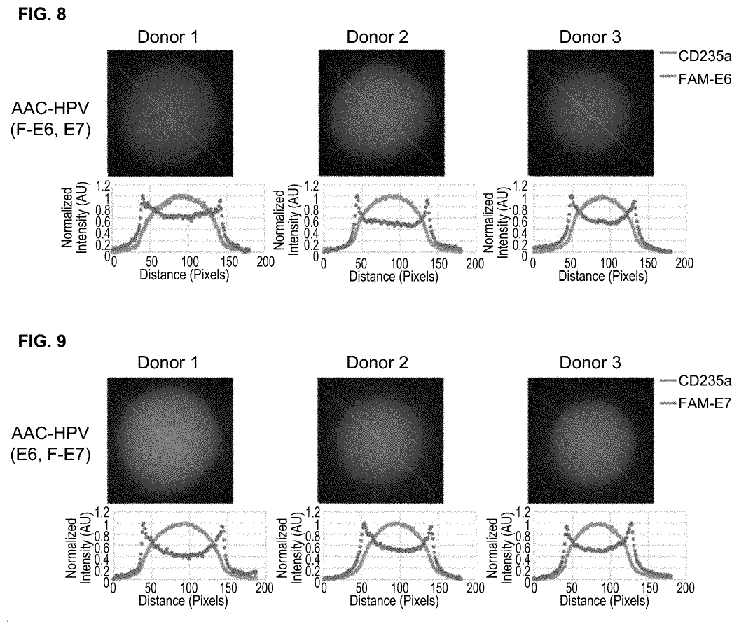

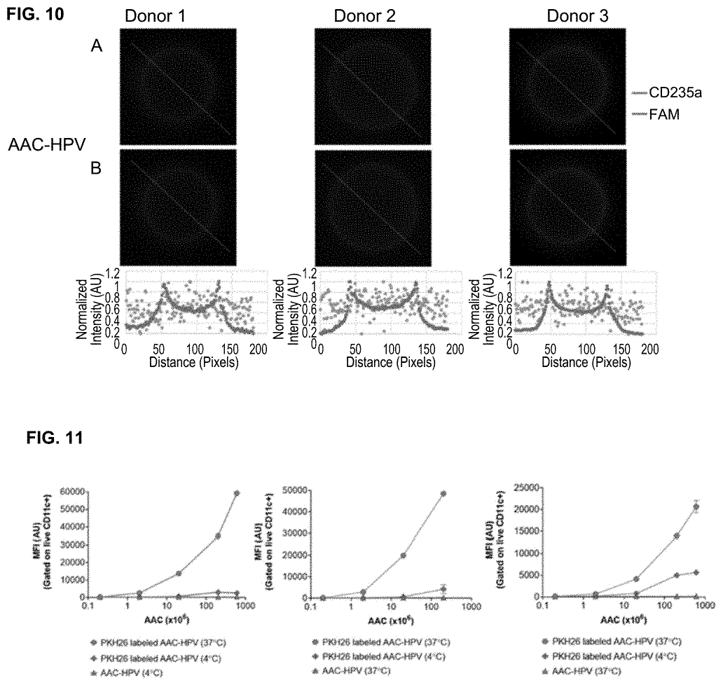

Representative epi-fluorescence images and their corresponding line scan traces for AAC-HPV (F-E6, E7), AAC-HPV (E6, F-E7), and AAC-HPV, generated by SQZ processing of three individual donor RBCs (1 donor per experiment), are shown in FIG. 8, FIG. 9 and FIG. 10, respectively. Means and ranges of the percentage of FAM+ AAC-HPV (F-E6, E7), AAC-HPV (E6, F-E7), and AAC-HPV are shown in Table 7.

TABLE 7
Mean Percentage of FAM⁺ AACs

| Group | Mean % FAM+ | Range of % FAM+ |
|---|---|---|
| AAC-HPV (F-E6, E7) | 100.0 | 100.0 |
| AAC-HPV (E6, F-E7) | 95.0 | 92.2-97.8 |
| AAC-HPV | 0.0 | 0.0 |

Table note: N = 3 donors/group.

Intracellular delivery of fluorescently labeled E6 and E7 SLPs (FAM-E6 and FAM-E7) into human AACs by the SQZ process was visualized via fluorescence microscopy. AAC-HPV (F-E6, E7), AAC-HPV (E6, F-E7), and AAC-HPV were stained with PB-conjugated anti-CD235a antibody to define the plasma membrane. Localization of FAM-E6 or FAM-E7 SLP was then visualized by fluorescence microscopy. AAC-HPV SQZ processed with unlabeled SLPs served as the negative control.

Line scans performed on fluorescent images confirmed the intra-AAC localization of FAM-E6 and FAM-E7 following SQZ processing. Specifically, intra-AAC FAM was observed in all (100.0%) AAC-HPV (F-E6, E7) analyzed and the majority (average of 95.0%) of AAC-HPV (E6, F-E7) analyzed.

This imaging study confirms the delivery of fluorescently labeled E6 or E7 SLPs into the majority of human AAC-HPV (F-E6, E7) and AAC-HPV (E6, F-E7), respectively, as the result of SQZ processing.

### Example 5. In Vitro Uptake of AAC-HPV by Human Antigen Presenting Cells (APCs) Measured by Flow Cytometry

The objective of study was to assess in vitro uptake of AAC-HPV by human antigen presenting cells (APCs).

Monocyte-derived dendritic cells (MODCs) generated from HLA-A*02+ donors by a five-day GM-CSF/IL-4 differentiation of CD14+ monocytes were used as an in vitro model of human APCs.

Red blood cells (RBCs) from 3 healthy human donors were labeled with PKH26, a lipophilic fluorescent membrane dye. Unlabeled and PKH26-labeled RBCs were SQZ processed with E6 SLP, E7 SLPs and poly I:C using the process described in Report No. SQZ-AAC-0124, generating unlabeled AAC-HPV and PKH26-labelled AAC-HPV, respectively.

In vitro uptake of AAC-HPV by MODCs was characterized as an increase in MODC (CD11c+ cells) PKH26 fluorescence after an overnight co-culture with PKH26-labeled AAC-HPV at 37° C., as measured by flow cytometry. Co-cultures of MODCs with PKH26-labeled AAC-HPV at 4° C. or MODCs with unlabeled AAC-HPV were used as negative controls. Summary graphs from three independent experiments are shown in FIG. 11. Summary data showing the fold increase in PKH26 fluorescence of MODCs cultured at 37° C. to MODCs co-cultured at 4° C. is shown in Table 8.

TABLE 8
Mean PKH26 Geometric Mean
Fluorescence Intensity (MFI) of MODCs Measured for
Different Doses of PKH26-labeled AAC-HPV for 3
Different MODC/RBC Donors for Co-cultures at 37° C. and 4° C.

| | AAC-HPV number | Mean MFI (4° C.) | Mean MFI (37° C.) |
|---|---|---|---|
| 966 | 0 | 239.00 | 274.00 |
| | 2 × 10⁶ | 295.50 | 2587.00 |
| | 20 × 10⁶ | 683.50 | 13698.00 |
| | 200 × 10⁶ | 3015.50 | 34860.50 |
| | 600 × 10⁶ | 2515.50 | 59232.50 |
| 1084 | 0 | 159.50 | 196.00 |
| | 2 × 10⁶ | 145.50 | 2874.50 |
| | 20 × 10⁶ | 638.50 | 19926.00 |
| | 200 × 10⁶ | 4218.50 | 48533.50 |
| | 600 × 10⁶ | N/A | N/A |
| 1574 | 0 | 148.50 | 142.50 |
| | 2 × 10⁶ | 192.00 | 672.67 |
| | 20 × 10⁶ | 802.67 | 4129.00 |
| | 200 × 10⁶ | 5005.33 | 13966.00 |
| | 600 × 10⁶ | 5631.33 | 20665.33 |

N/A denotes not available. In the ELN 1084, the highest tested dose of PKH26-labeled AAC-HPV or AAC-HPV was 200 x 10⁶ cells per well.

PKH26 MFI of MODCs co-cultured with PKH26-labeled AAC-HPV at 37° C. showed an increase (2.8-31.2-fold) over PKH26 MFI of MODCs co-cultured at 4° C., a temperature where uptake is depressed (Albert 1998). This was observed for AAC-HPV doses ranging from 2-600×10⁶ in all studies (3 of 3 experiments). Fluorescence was not observed in co-cultures including unlabeled AAC-HPV, demonstrating that the increase in PKH26 MFI of MODCs is dependent on PKH26-labeled AAC-HPV. Thus, CD11c+ MODCs internalize PKH26-labeled AAC-HPV in a dose- and temperature-dependent manner.

This study demonstrates that MODCs take up PKH26-labeled AAC-HPV in a dose- and temperature-dependent manner.

### Example 6. In Vitro Maturation of APCs Following AAC-HPV Uptake as Measured by Flow Cytometry

The objective of this study was to assess the in vitro upregulation of maturation markers on the human model APCs, MODCs, (monocyte-derived dendritic cells), following approximately two days of co-culture with AAC-HPV.

Monocytes from each of five HLA-A*02+ donors were incubated with GM-CSF/IL-4 for 4 days to generate five lots of MODCs. MODCs were phenotyped, frozen and stored at ≤140° C. until thawed for use.

Human RBCs were SQZ processed with E6 SLP, E7 SLP and poly I:C using the process described in Report No. SQZ-AAC-0124, generating AAC-HPV. Similarly, human RBCs were SQZ processed with media in the absence of the antigens (E6 and E7 SLPs) and adjuvant (poly I:C) to generate C-media.

MODCs from five different donors were co-cultured for approximately two days with AAC-HPV.

The upregulation of maturation markers was determined by measuring the geometric mean fluorescence intensity (MFI) of CD86, CD80, CD83, MHC-II and CD40 staining by flow cytometry, and comparing it to the maturation marker levels of MODCs cultured with C-media or control media alone.

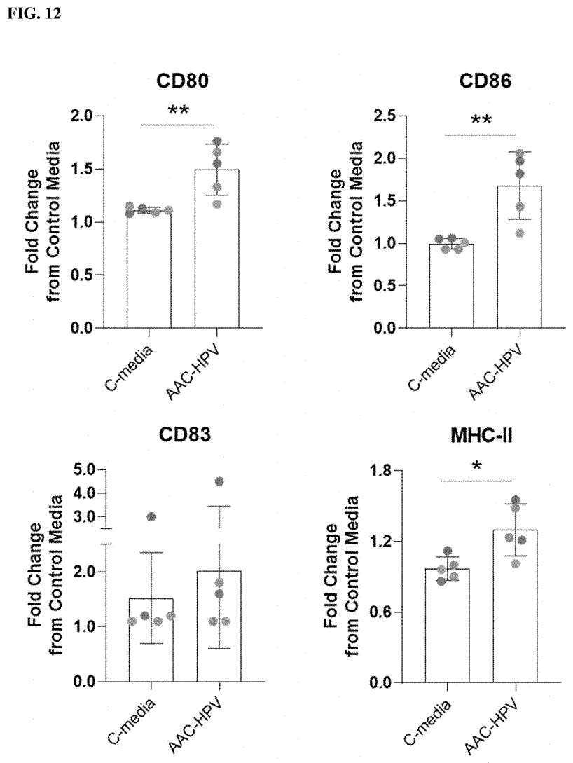

Summary graphs for CD86, CD80, CD83 and, MHC-II are shown in FIG. 12. AAC-HPV co-cultured with MODCs did not result in an increase in CD40 expression on the MODCs relative to the culture with the control media; therefore, CD40 is not presented in a graph.

A statistically significant increase in upregulation of maturation markers on the MODC surface was observed for CD80, CD86 and MHC-II. Although a statistically significant increase in upregulation of the maturation marker CD83 was not observed, three of five MODC donors exhibited an upregulation of CD83 following co-culture with AAC-HPV compared to C-media. In addition, statistical analysis performed on raw (non-normalized) data of control media, C-media and AAC-HPV showed no difference between control media and C-media confirming that RBCs SQZ processed without the adjuvant (and antigens) do not upregulate maturation markers on MODCs.

This study demonstrates that MODCs co-cultured in vitro with AAC-HPV significantly upregulates multiple maturation markers including CD80, CD86, and MHC-II on the surface of MODCs. While not significant, the upregulation of CD83 is observed in 3 of 5 donors used for MODC generation.

### Example 7. In Vitro Activity of SQZ-AAC-HPV Measured as IFN Secretion by E711-20 Reactive CD8⁺ T Cells Following Co-Culture with MODCs

The objective of study was to demonstrate a functional response to SQZ-AAC-HPV co-cultured with human model APCs, MODCs, (monocyte-derived dendritic cells), and E711-20 specific CD8⁺ T cells.

Seven different batches of SQZ-AAC-HPV were generated by SQZ processing healthy donor fresh blood with E6 and E7 SLPs and poly I:C and were formulated as the drug product. SQZ-AAC-HPV was co-cultured with MODCs derived from an HLA-A*02+ donor by a five-day stimulation of CD14+ monocytes with GM-CSF and IL-4. Media from the resultant co-cultures were analyzed by ELISA for IFN secretion from E711-20 specific CD8⁺ T cells.

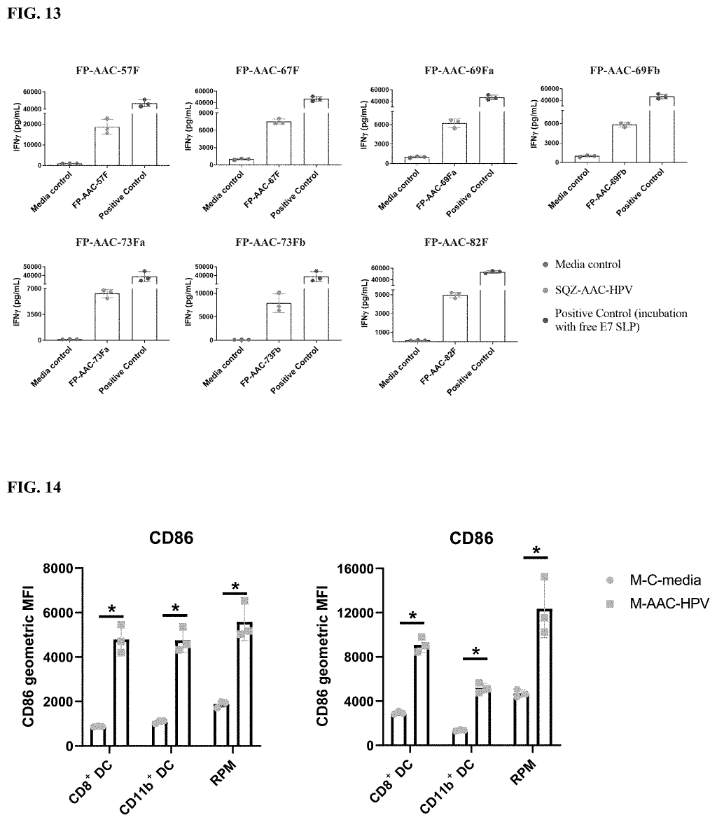

Summary data from seven different lots showing SQZ-AAC-HPV induced antigen specific IFN responses from E7-specific CD8⁺ T cells co-cultured with MODCs as measured by ELISA are shown in FIG. 13. Summary data showing the magnitude of IFN secretion presented as a fold increase between IFN measured in the SQZ-AAC-HPV-containing co-cultures and IFN measured in the media control co-culture is shown in Table 9.

Co-cultures of MODCs and CD8⁺ T cells with all 7 batches of SQZ-AAC-HPV resulted in at least a 6-fold increase in secreted IFNγ compared to co-cultures of MODCs and CD8⁺ T cells with media control.

This study demonstrates that the SQZ-AAC-HPV induces secretion of IFNγ by E7-specific CD8⁺ T cells that recognize the E711-20 minimal epitope following in vitro co-culture with HLA-A*02+ MODCs and E7-specific CD8⁺ T cells.

TABLE 9
Activity of SQZ-AAC-HPV is Measured in MODC and E7-specific CD8+ T Cell Co-
cultures for 7 Independent SQZ-AAC-HPV Batches

| | | Mean Media | Mean SQZ-AAC- | Fold Increase of IFNy |
|---|---|---|---|---|
| | | Control | HPV | of SQZ-AAC-HPV |
| ELN | SQZ-AAC-HPV batch | (IFNy pg/mL) | (IFNy pg/mL) | over Media Control⁸ |
| 1805 | FP-AAC-57F | 990.8 | 18654.3 | 18.8 |
| | FP-AAC-67F | 990.8 | 7513.7 | 7.6 |
| | FP-AAC-69Fa | 990.8 | 6194.5 | 6.3 |
| | FP-AAC-69Fb | 990.8 | 5843.7 | 5.9 |
| 1868 | FP-AAC-73Fa | 136.3 | 6329.2 | 46.4 |
| | FP-AAC-73Fb | 136.3 | 7910.5 | 58.1 |
| 1903 | FP-AAC-82F | 150.7 | 4955.6 | 32.9 |

Told increase is calculated for SQZ-AAC-HPV and media control condition

### Example 8. In Vivo Maturation of the Endogenous APCs in Mice Following Intravenous Administration of M-AAC-HPV as Measured by Flow Cytometry

The objective of the study SQZ-AAC-0127 was to assess the in vivo upregulation of maturation markers on various endogenous splenic APCs (antigen presenting cells) after immunization of mice with the mouse prototype, M-AAC-HPV.

Table 10 illustrates the design of the study to evaluate the activation of splenic APCs by M-AAC-HPV in vivo in female C57BL/6J mice. M-C-media (mouse RBCs SQZ processed with media (in the absence of antigen or adjuvant) was used as a control. The day of the animal sacrifice is the day immunophenotyping was performed.

The splenic APCs evaluated were CD11chiMHC-IIhiCD8+ cells (CD8+ dendritic cells or CD8+ DC), CD11chiMHC-IIhiCD11b+ cells (CD11b+ dendritic cells or CD11b+ DC), and F4/80+ CD11blo/− (RPM; red pulp macrophages).

The upregulation of APC maturation markers was demonstrated by measuring the geometric mean fluorescence intensity (MFI) of CD40, CD86, CD80, CD83 and MHC-II staining by flow cytometry. Flow cytometry analysis of the spleen was performed 14-16 hours after administration of M-AAC-HPV or M-C-media to allow the accumulation of maturation markers on the cell surface.

TABLE 10
In Vivo Antigen Presenting Cell Maturation by M-AAC-HPV

| | | | M-C-media/ | | | |
|---|---|---|---|---|---|---|
| | | M-C-media/ | M-AAC-HPV | | Study Schedule | |
| | | M-AAC-HPV | Final Concentration | Total Volume | Time = | Time = |
| Groups | Mice (#) | Dose (#/Mouse) | for Injection (#/mL) | Injected (μL) | 0 hour | 14-16 hours |
| A: M-C-media | 3 | 1 × 10⁹ | 5 × 10⁹ | 200 | RO | Harvest |
| | | | | | injection | spleen |
| B: M-AAC-HPV | 3 | 1 × 10⁹ | 5 × 10⁹ | 200 | RO | Harvest |
| | | | | | injection | spleen |

M-AAC-HPV = Mouse Prototype of AAC-HPV (Mouse RBCs SQZ Processed with Mouse E7 SLP and Poly I:C); M-C-media = Mouse RBCs SQZ processed with media (in the absence of antigen or adjuvant); RO: retro-orbitally (route of administration)

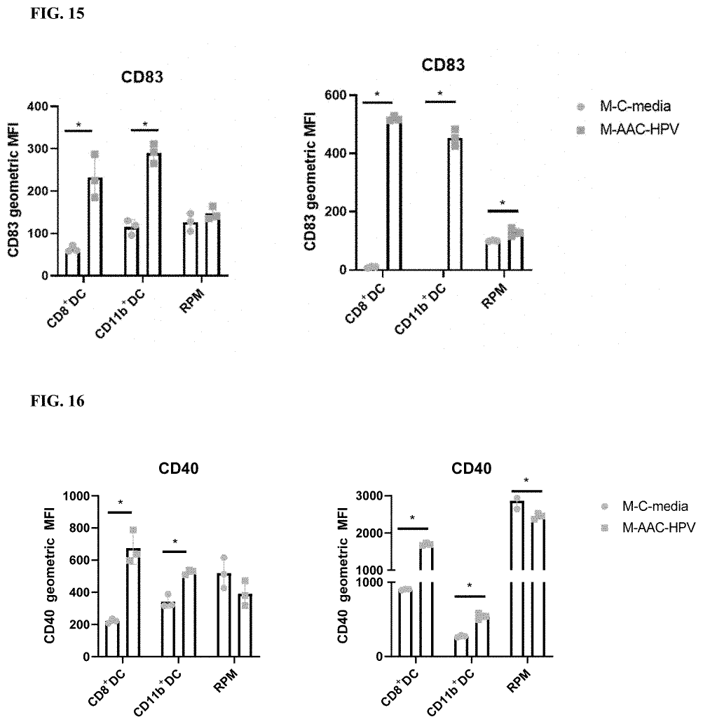

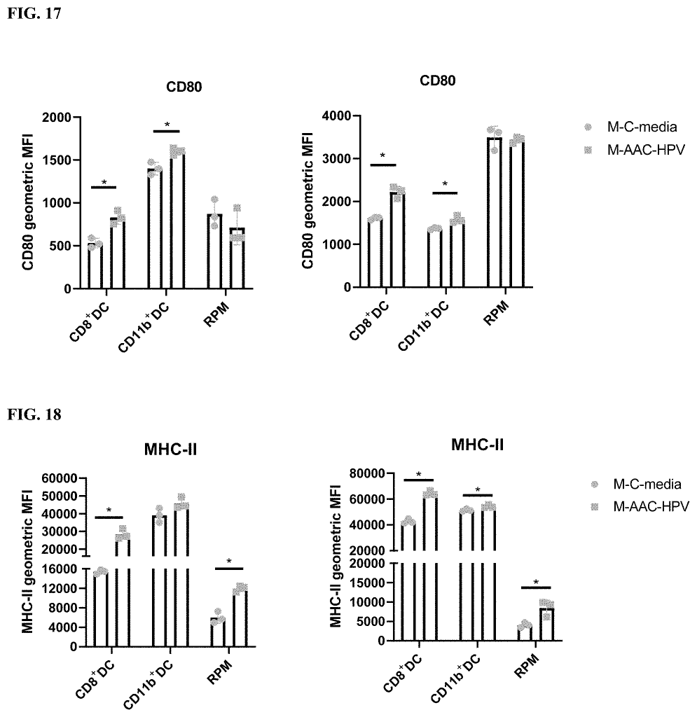

Summary graphs for markers on splenic APCs are shown in FIG. 14 for CD86 geometric MFI, in FIG. 15 for CD83 geometric MFI, in FIG. 16 for CD40 geometric MFI, in FIG. 17 for CD80 geometric MFI, and in FIG. 18 for MHC-II geometric MFI.

Results from two independent experiments demonstrated a statistically significant increase in CD86 geometric MFI on all three splenic APC populations (CD8+ DC, CD11b+ DC, RPM) in mice that received M-AAC-HPV compared to mice that received M-C-media. Results from 2 independent experiments demonstrated a statistically significant increase in CD83, CD40 and CD80 geometric MFI, selectively, on splenic dendritic cells, namely, CD8+ DC and CD11b+DC in mice that received M-AAC-HPV compared to mice that received M-C-media. Results from 2 independent experiments demonstrated a statistically significant increase in MHC-II geometric MFI, selectively, on splenic CD8⁺ DC and RPM in mice that received M-AAC-HPV compared to mice that received M-C-media.

The study demonstrated that immunization of mice with the mouse prototype M-AAC-HPV activates splenic APCs including CD8+ DCs, CD11b+ DCs and RPMs in vivo. Upregulation of co-stimulatory markers (CD86, CD83, CD40, CD80, and MHC-II), markers for maturation, was observed on the various APC populations 14-16 hours post intravenous (IV) administration of M-AAC-HPV, but not in mice that received mouse RBCs that were SQZ processed without any antigen or adjuvant (M-C-media).

### Example 9. In Vivo Immunization in Mice with M-AAC-HPV to Measure CD8⁺ T Cell Endogenous Responses as Assessed by Flow Cytometry ICS—Effect of Antigen and Adjuvant

The requirement for antigen and adjuvant in priming E7-specific CD8⁺ T cell response in mice was examined using intracellular cytokine staining (ICS) for IFNγ.

Harvest of mouse blood and isolation of RBCs was performed as described above. RBCs were resuspended and SQZ processed with a PBS solution containing either E7 SLP alone (to generate M-AC) or poly I:C alone (to generate M-C-poly I:C) or a solution containing both E7 SLP and poly I:C (to generate M-AAC-HPV), with SQZ processing conditions as described in report SQZ-AAC-0126.

Table 11 illustrates the design of the study to evaluate the requirement for antigen (E7 SLP) and adjuvant (poly I:C) in SQZ processed red blood cells (RBCs) to elicit an E7-specific CD8⁺ T cell response in vivo in female C57BL/6J mice. The day of the animal sacrifice is the day intracellular cytokine staining (ICS) was performed on splenocytes.

TABLE 11
Effect of Antigen and Adjuvant on Endogenous Responses

| | | Dose Per | Final concentration | Total Volume | Study Schedule | |
|---|---|---|---|---|---|---|
| Groups | Mice (#) | Mouse (#/Mouse) | for Injection (#/mL) | Injected (μL) | Day 0 | Day 7 |
| A: Vehicle (PBS) | 5 | NA | NA | 200 | RO | ICS |
| | | | | | inject | |
| B: M-C-poly I:C | 5 | 250 × 10⁶ | 1.25 × 10⁹ | 200 | RO | ICS |
| | | | | | inject | |
| C: M-AC | 5 | 250 × 10⁶ | 1.25 × 10⁹ | 200 | RO | ICS |
| | | | | | inject | |
| D: M-AAC-HPV | 5 | 250 × 10⁶ | 1.25 × 10⁹ | 200 | RO | ICS |
| | | | | | inject | |

ICS = intracellular cytokine staining; M-AAC-HPV = Mouse Prototype of AAC-HPV (Mouse RBCs SQZ Processed with Mouse E7 SLP and Poly I:C); M-AC = Mouse RBCs SQZ processed with antigen alone (in the absence of adjuvant); M-C-poly I:C = Mouse RBCs SQZ processed with adjuvant alone (in the absence of antigen); NA = not applicable; PBS = phosphate buffered saline; RO = retro-orbitally (method of IV administration)

On the day indicated in Table 11 above for ICS, the mice were sacrificed, the spleens collected, and cells were isolated for analysis. The E7-specific CD8⁺ T cell response is measured by evaluating the percentage of CD8⁺ T cells that produce IFNγ when restimulated with the E7 minimal epitope peptide.

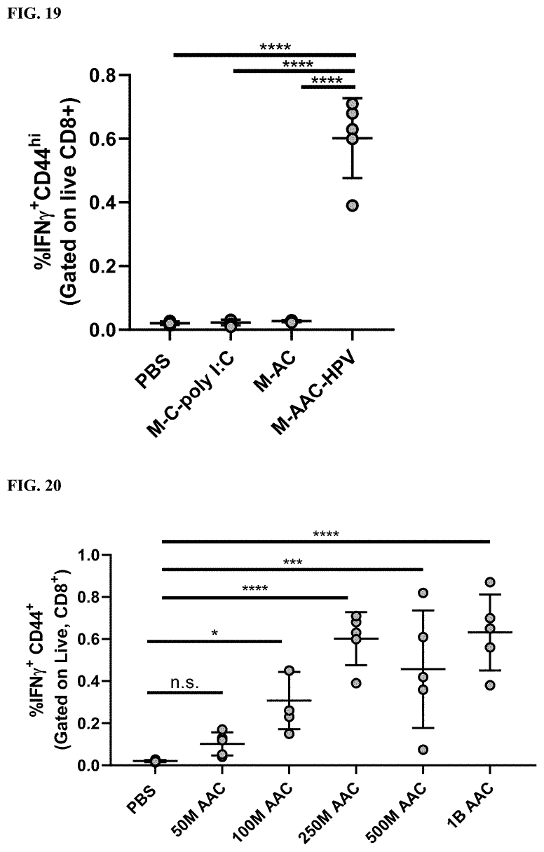

The magnitude of E7-specific CD8⁺ T cell responses is shown in FIG. 19. The percentage of IFNγ-producing CD8⁺ T cells is greatest when the SQZ processed cells used for immunization contain both adjuvant (poly I:C) and antigen (E7 SLP). The percentage of CD8⁺ T cells that produce IFNγ when restimulated with the E7 minimal epitope peptide in animals that received M-AAC-HPV was significantly greater (p<0.0001) than in animals that received SQZ processed RBCs prepared with adjuvant alone (M-C-poly I:C) or antigen alone (M-AC) (a mean of 0.60% for M-AAC-HPV compared to 0.02% for M-C-poly I:C and 0.03% for M-AC).

Mice treated with M-AAC-HPV elicited significant E7 specific CD8⁺ T cell responses, which are dependent on the presence of antigen (E7) and adjuvant (poly I:C).

### Example 10. Assessment of CD8⁺ T Cell Endogenous Responses in Mice Following M AAC-HPV Administration—Dose Response

The objective of this study was to examine the effect of increasing doses of M-AAC-HPV (5×10⁷, 1×10⁸, 2.5×10⁸, 5×10⁸, and 1×10⁹ M-AAC HPV/mouse) on the E7-specific CD8⁺ T cell response in mice using intracellular cytokine staining (ICS) for IFNγ.

The methods for isolating mouse RBCs and SQZ processing RBCs with E7 SLP and poly I:C to generate the mouse prototype of AAC-HPV (M-AAC-HPV) are described above.

Table 12 illustrates the design of the study to evaluate different doses of M-AAC-HPV in vivo. Female C57BL/6J mice were used for studies. The day of the animal sacrifice is the day intracellular cytokine staining (ICS) was performed on splenocytes.

TABLE 12
M-AAC-HPV Dose Titration

| | | M-AAC-HPV | M-AAC-HPV | | | |
|---|---|---|---|---|---|---|
| | | Dose Per | final concentration | Total Volume | Study Schedule | |
| Groups | Mice (#) | Mouse (#/Mouse) | for injection (#/mL) | Injected (μL) | Day 0 | Day 7 |
| A: Vehicle (PBS) | 5 | NA | NA | 200 | RO inject | ICS |
| B: 50 M M-AAC-HPV | 5 | 5 × 10⁷ | 2.5 × 10⁸ | 200 | RO inject | ICS |
| C: 100 M M-AAC-HPV | 5 | 1 × 10⁸ | 5 × 10⁸ | 200 | RO inject | ICS |
| D: 250 M M-AAC-HPV | 5 | 2.5 × 10⁸ | 1.25 × 10⁹ | 200 | RO inject | ICS |
| E: 500 M M-AAC-HPV | 5 | 5 × 10⁸ | 2.5 × 10⁹ | 200 | RO inject | ICS |
| F: 1 B M-AAC-HPV | 5 | 1 × 10⁹ | 5 × 10⁹ | 200 | RO inject | ICS |

M denotes million; B denotes billion; NA denotes “not applicable”; RO: retro-orbitally (method of IV administration); ICS = intracellular cytokine staining

On the day indicated in the table above for ICS, the mice were sacrificed, the spleens collected, and cells were isolated for analysis. The E7-specific CD8+ T cell response is measured by evaluating the percentage of CD8+ T cells that produce IFNγ when restimulated with the E7 minimal epitope peptide. The magnitude of the E7-specific CD8+ T cell response shown in FIG. 20 demonstrates the effect of a range of doses of M-AAC-HPV.

As seen in FIG. 20, the magnitude of the E7-specific CD8+ T cell IFN□ response is dependent on the dose of M-AAC-HPV. As the dose of M-AAC-HPV increases, the response increases accordingly, with the percentage of CD8+ T cells that produce IFNγ when re-stimulated with the E7 short peptide increasing from a mean value of 0.10% at a dose of 5×10⁷ M-AAC-HPV/mouse to a mean value of 0.63% at a dose of 1×10⁹ M-AAC-HPV/mouse, compared with the mean value for the PBS control of 0.02%. Over the dose range studied (5×10⁷ M-AAC-HPV/mouse to 1×10⁸ M-AAC-HPV/mouse), the response appeared to plateau at 2.5×10⁸ M-AAC-HPV/mouse.

Mice treated with M-AAC-HPV elicited significant CD8⁺ T cell IFNγ responses, the magnitude of which is dependent on the M-AAC-HPV dose.

### Example 11. In Vivo Immunization in Mice with M-AAC-HPV to Measure E7-Specific CD8⁺ Cells in Blood as Assessed by Tetramer Staining—Effect of Boosting Schedule

The objective of this was to determine the effect of booster administrations of M-AAC-HPV to mice on the magnitude of the E7-specific CD8+ T cell endogenous response as measured in blood using tetramer staining.

Table 13 illustrates the design of the study to evaluate the impact of administering additional booster doses of M-AAC-HPV to female C57BL/6J mice on the magnitude of the E7-specific CD8⁺ T cell response. The E7-specific CD8⁺ T cells were measured by staining cells in whole blood with MHC Class I tetramers that bind T cell receptors (TCRs) specific for the E7 immunodominant epitope in mice (E749-57-RAHYNIVTF), and evaluating the percentage of E7 tetramer+ CD8⁺ T cells in whole blood by flow cytometry.

TABLE 13
Effect of Boosting on E7-Specific CD8+ T Cell Responses

| | | | | | | | | Tetramer analysis |
|---|---|---|---|---|---|---|---|---|
| | | | | | | | | time points |
| | | | | | | | | (# of days |
| | | M-AAC-HPV | M-AAC-HPV | | | | | after the final |
| | | dose per mouse | concentration for | Total volume | Immunization study schedule | | | immunization) |
| Groups | Mice (#) | (#/mouse) | injection (#/mL) | injected (μL) | Day 0 | Day 2 | Day 6 | (study days) |
| PBS | 5 | NA | NA | 200 | RO | NA | NA | 8, 13, 21 |
| | | | | | inject | | | (8, 13, 21) |
| M-AAC-HPV- | 5 | 0.25 × 10⁹ | 1.25 × 10⁹ | 200 | RO | NA | NA | 8, 13, 21 |
| Prime alone | | | | | inject | | | (8, 13, 21) |
| M-AAC-HPV- | 5 | 0.25 × 10⁹ | 1.25 × 10⁹ | 200 | RO | RO | NA | 8, 13, 21 |
| Day 2 boost | | | | | inject | inject | | (10, 15, 23) |
| M-AAC-HPV- | 5 | 0.25 × 10⁹ | 1.25 × 10⁹ | 200 | RO | NA | RO | 7, 14 |
| Day 6 boost | | | | | inject | | inject | (13, 21) |

NA denotes “not applicable”; RO: retro-orbitally (method of IV administration)

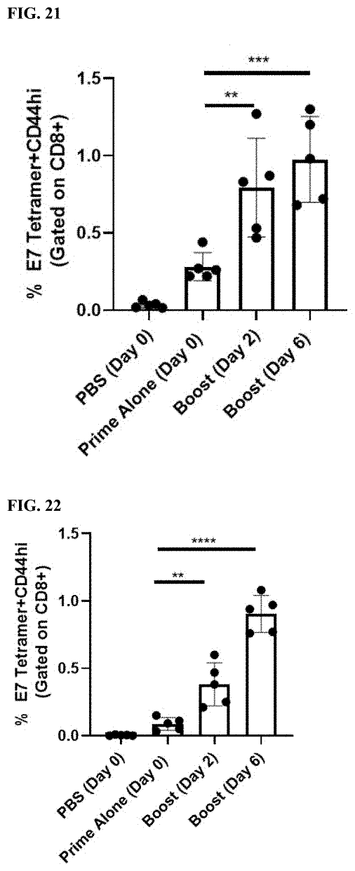

The magnitude of E7-specific CD8⁺ T cell responses at various time points after the last immunization are shown in FIGS. 21, 22 and 23.

In this study, CD8+ T cell responses to E7 over time were monitored by measuring the percentage of activated (CD44^{hi}) E7-tetramer positive CD8⁺ T cells relative to all CD8⁺ T cells in whole blood. As seen in FIGS. 21-23, the administration of a single dose of M-AAC-HPV primed an E7-specific CD8⁺ T cell response as evidenced by an increase in E7 tetramer+CD8⁺ T cells in whole blood in the prime only group (range 0.22%-0.44%, mean 0.28%) compared to the PBS controls (range 0.02%-0.07%, mean 0.03%), although this difference did not reach statistical significance. Furthermore, the percentage of E7 tetramer+CD8⁺ T cells could be significantly increased by additional immunizations administered either 2 days or 6 days after the prime relative to animals that received only the priming dose.

The maximal E7 tetramer⁺ CD8⁺ T cell response for all groups was observed approximately one week after the final immunization. The percentage of E7-specific CD8⁺ T cells ranged from 0.02%-0.07% for the PBS control group (mean 0.03%), 0.22%-0.44% (mean 0.28%) for the animals in the prime alone group, 0.47%-1.27% (mean 0.79%) for the animals boosted on day 2 and between 0.68%-1.30% (mean 0.98%) for the animals boosted on day 6. By approximately two weeks after the final immunization, the E7-specific CD8+ T cell response in animals that received a single priming dose (range 0.03%-0.11%) was not significantly different from PBS controls (range 0.00%-0.03%). In contrast, the E7-specific CD8⁺ T cell response in animals that were boosted on either day 2 (range: 0.21%-0.6%) or day 6 (range 0.76%-1.08%) remained elevated and significantly greater than the response in the prime alone animals. Furthermore, animals that were boosted on day 6 had significantly greater E7-specific CD8⁺ T cells even compared to animals that were boosted on day 2.

The study herein demonstrates that immunization with M-AAC-HPV primes E7-specific CD8⁺ T cell responses in vivo in blood. Booster doses of 250×10⁶ M-AAC-HPV administered intravenously to mice 2 days or 6 days after a priming dose of 250×10⁶ M-AAC-HPV resulted in a significant and sustained increase in E7-specific CD8⁺ T cell responses relative to both the PBS control group and the prime-only group.

### Example 12. In Vivo Determination of Efficacy in Mice Following Therapeutic Immunization with Intravenously Administered M-AAC-HPV in the TC 1 Tumor Model—Requirement for Antigen

The objective of this study was to assess the requirement for antigen to inhibit tumor growth and extend median survival in a therapeutic TC-1 tumor model following a single vaccination of intravenously administered M-AAC-HPV at 250×10⁶ or 1×10⁹ M-AAC-HPV per mouse compared to a single intravenous administration of M-C-poly I:C (mouse RBCs SQZ processed with poly I:C (no antigens)) at the same doses and to intravenous PBS administration.

The methods for isolating mouse RBCs and SQZ processing RBCs with E7 SLP and poly I:C to generate the mouse prototype of AAC-HPV (M-AAC-HPV) are described in Report No. SQZ-AAC-0126. M-C-poly I:C was generated equivalently using SQZ processing of mouse RBCs with poly I:C.

The following tables illustrate the design of the studies to evaluate the effect of antigen in the tumor studies (Table 14 and Table 15). The mice (female C57BL/6J) were injected subcutaneously (SC) with the TC-1 tumor cells (50,000 cells) on Day 0. The mice were treated with the test articles by retro-orbital administration on the day described in Table 14 and Table 15. Survival was monitored daily, and tumor growth was measured twice a week.

TABLE 14
Anti-Tumor Effect of Antigen in M-AAC-HPV
ELN103212

| | | | | M-AAC-HPV or | | | |
|---|---|---|---|---|---|---|---|
| | | | M-AAC-HPV | M-C-poly I:C | | | |
| | | | or M-C-poly | final concentration | Total | | |
| | | | I:C Dose Per | for injection | Volume | TC-1 | Administration |
| Groups | | Mice (#) | Mouse (Number) | (AAC or C/mL) | Injected | Implant | of Test Article |
| A | PBS | 10 | NA | NA | 200 μL | Day 0 | Day 10 |
| B | 250 M M-C-poly I:C | 10 | 250 × 10⁶ | 1.25 × 10⁹ | 200 μL | Day 0 | Day 10 |
| C | 250 M M-AAC-HPV | 10 | 250 × 10⁶ | 1.25 × 10⁹ | 200 μL | Day 0 | Day 10 |

NA denotes “not applicable”. ‘M’ denotes million.

TABLE 15
Anti-Tumor Effect of Antigen in M-AAC-HPV
ELN1416

| | | | | M-AAC-HPV or | | | |
|---|---|---|---|---|---|---|---|
| | | | M-AAC-HPV | M-C-poly I:C | | | |
| | | | or M-C-poly | final concentration | Total | | |
| | | | I:C Dose Per | for injection | Volume | TC-1 | Administration |
| Groups | | Mice (#) | Mouse (Number) | (AAC or C/mL) | Injected | Implant | of Test Article |
| A | PBS | 10 | NA | NA | 100 μL | Day 0 | Day 14 |
| B | 1 B M-C-poly I:C | 10 | 1 × 10⁹ | 5 × 10⁹ | 200 μL | Day 0 | Day 14 |
| C | 1 B M-AAC-HPV | 10 | 1 × 10⁹ | 5 × 10⁹ | 200 μL | Day 0 | Day 14 |

NA denotes “not applicable”. ‘B’ denotes billion.

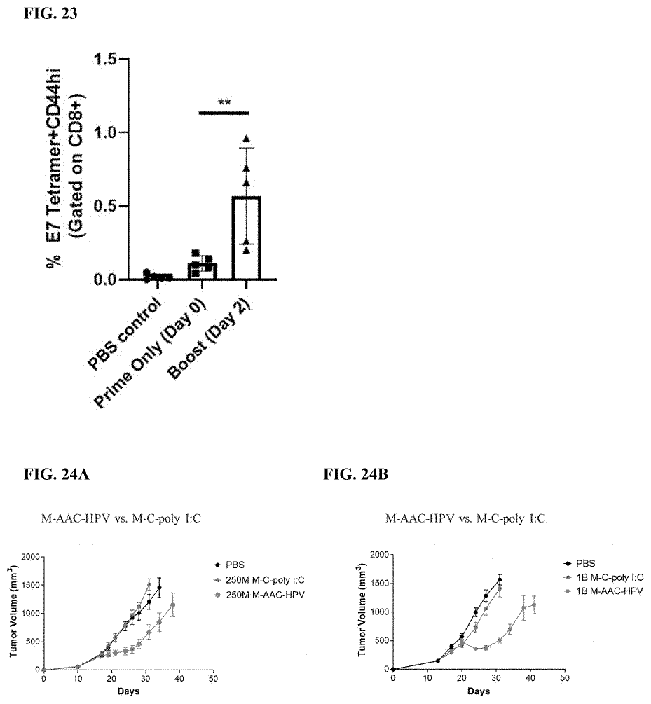

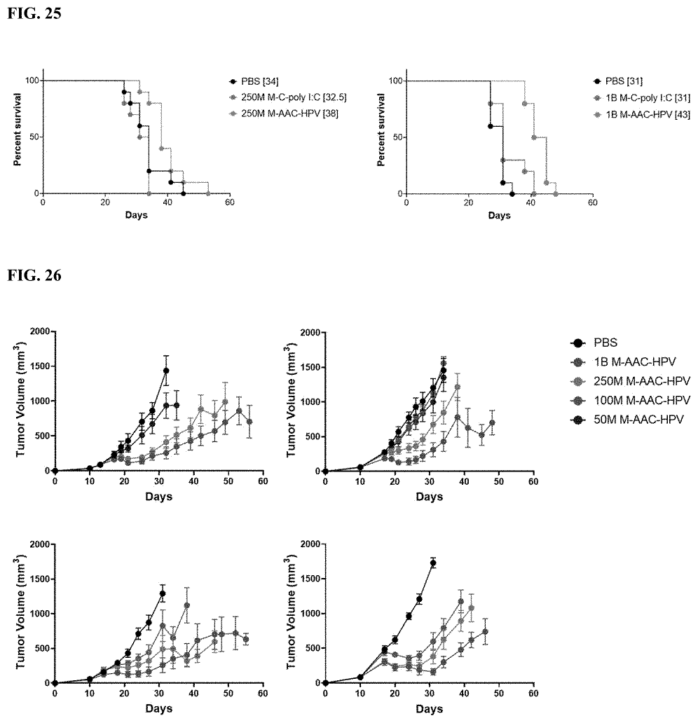

The summary tumor growth data from two independent experiments (ELN103212 and ELN1416) are shown in FIG. 23 and the summary survival data are shown in FIG. 25 and Table 16.

TABLE 16
Median Survival Times of Groups Treated
with M-AAC-HPV or M-C-poly I:C
Median Survival (Days)

| ELN | Group | Days |
|---|---|---|
| 103212 | PBS | 34.0 |
| | 250M M-C-poly I:C | 32.5 |
| | 250M M-AAC-HPV | 38.0 |
| 1416 | PBS | 31.0 |
| | IB M-C-poly I:C | 31.0 |
| | IB M-AAC-HPV | 43.0 |

As shown in FIGS. 24A and 24B, an intravenously administered M-AAC-HPV dose of 250×10⁶ or 1×10⁹ significantly delayed tumor growth rates in both studies. An administration of an equivalent dose of adjuvant carriers without antigen, M-C-poly I:C, failed to slow tumor growth relative to PBS controls.

Mice treated with either dose of M-AAC-HPV showed a statistically significant prolonged survival compared to control-treated mice as seen FIG. 25 and Table 16. Survival ranged from 38 to 53 days in mice treated with 250×10⁶ M-AAC-HPV (study ELN103212), and from 38 to 48 days in mice treated with 1×10⁹ M-AAC-HPV (study ELN1416). In mice treated with PBS and 250×10⁶ M-C-poly I:C survival ranged from 26 to 45 days, and 26 to 34 days, respectively (ELN103212). In the 2nd study (ELN1416), survival ranged from 27 to 34 days, and 27 to 41 days for PBS and 1×10⁹ M-C-poly I:C treated mice.

Treatment of mice with an intravenously administered dose of 250×10⁶ or 1×10⁹ M-AAC-HPV resulted in a significantly delayed tumor growth compared to control-treated mice. In addition, M-AAC-HPV treated mice showed statistically significant extended survival at both doses. In contrast, mice treated with M-C-poly I:C showed no improvement in delaying tumor growth or extending survival relative to controls, irrespective of M-C-poly I:C dose.

These data support the necessity of antigen presence for efficacy in the therapeutic TC-1 model.

### Example 13. In Vivo Determination of Therapeutic Efficacy in Mice Following Therapeutic Immunization with Intravenously Administered M-AAC-HPV in the TC 1 Tumor Model—Dose Response

The objective of this study was to assess the anti-tumor activity of increasing doses of intravenously administered M-AAC-HPV (50×10⁶, 100×10⁶, 250×10⁶, and 1×10⁹ M-AAC-HPV/mouse) in the TC-1 mouse tumor model.

The methods for isolating mouse RBCs and SQZ processing RBCs with E7 SLP and poly I:C to generate the mouse prototype of AAC-HPV (M-AAC-HPV) are described above.

The following tables illustrate the design of the studies to evaluate different doses of M-AAC-HPV (Table 17 through Table 20) in the tumor studies. The mice (female C57BL/6J) were injected subcutaneously (SC) with the TC-1 tumor cells (50,000 cells) on Day 0. On Day 10 of the study, the mice were treated with the test articles by retro-orbital administration. Survival was monitored daily, and tumor growth was measured twice a week.

TABLE 17
M-AAC-HPV Dose Titration for ELN 103 05 8
ELN103058

| | | | M-AAC-HPV Dose | M-AAC-HPV final | Total Volume |
|---|---|---|---|---|---|
| | | | Per Mouse | concentration for | Injected |
| Groups | | Mice (#) | (#/Mouse) | injection (#/mL) | (uL) |
| A | PBS | 10 | NA | NA | 200 |
| B | M-AAC-HPV | 10 | 1 × 10⁹ | 5 × 10⁹ | 200 |
| C | M-AAC-HPV | 10 | 250 × 10⁶ | 1.25 × 10⁹ | 200 |
| D | M-AAC-HPV | 10 | 50 × 10⁶ | 250 × 10⁶ | 200 |

NA denotes “not applicable”

TABLE 18
M-AAC-HPV Dose Titration for ELN103212
ELN103212

| | | | M-AAC-HPV Dose | M-AAC-HPV final | Total Volume |
|---|---|---|---|---|---|
| | | | Per Mouse | concentration for | Injected |
| Groups | | Mice (#) | (#/Mouse) | injection (#/mL) | (uL) |
| A | PBS | 10 | NA | NA | 200 |
| B | M-AAC-HPV | 10 | 1 × 10⁹ | 5 × 10⁹ | 200 |
| C | M-AAC-HPV | 10 | 250 × 10⁶ | 1.25 × 10⁹ | 200 |
| D | M-AAC-HPV | 10 | 100 × 10⁶ | 500 × 10⁶ | 200 |
| E | M-AAC-HPV | 10 | 50 × 10^{s} | 250 × 10⁶ | 200 |

NA denotes “not applicable”

TABLE 19
M-AAC-HPV Dose Titration for ELN133 (SQZ-AAC-0132)
ELN133

| | | | M-AAC-HPV Dose | M-AAC-HPV final | Total Volume |
|---|---|---|---|---|---|
| | | | Per Mouse | concentration for | Injected |
| Groups | | Mice (#) | (#/Mouse) | injection (#/mL) | (uL) |
| A | PBS | 10 | NA | NA | 200 |
| B | M-AAC-HPV | 10 | 1 × 109 | 5 × 10⁹ | 200 |
| C | M-AAC-HPV | 10 | 250 × 10⁶ | 1.25 × 10⁹ | 200 |
| D | M-AAC-HPV | 10 | 100 × 10⁶ | 500 × 10⁶ | 200 |

NA denotes “not applicable”

TABLE 20
M-AAC-HPV Dose Titration for ELN1069
ELN1069

| | | | M-AAC-HPV | M-AAC-HPV final | Total Volume |
|---|---|---|---|---|---|
| | | | Dose Per Mouse | concentration for | Injected |
| Groups | | Mice (#) | (#/Mouse) | injection (#/mL) | (uL) |
| A | PBS | 10 | NA | NA | 100 |
| B | M-AAC-HPV | 10 | 1 × 10⁹ | 5 × 10⁹ | 200 |
| C | M-AAC-HPV | 10 | 250 × 10⁶ | 2.5 × 10⁹ | 100 |
| D | M-AAC-HPV | 10 | 100 × 10⁶ | 1 × 10⁹ | 100 |

NA denotes “not applicable”

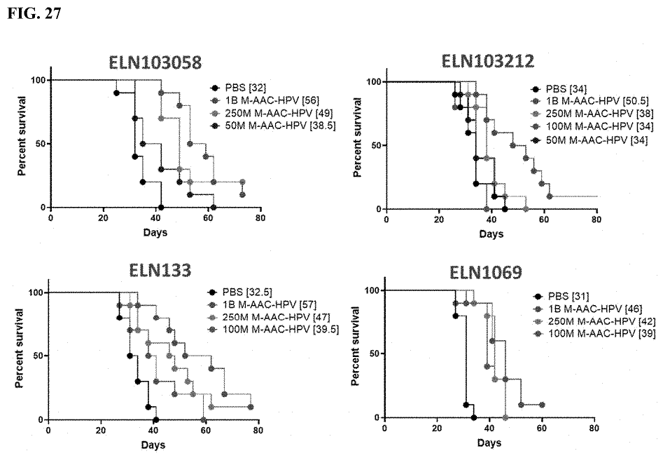

The summary tumor growth data from 4 independent experiments are shown in FIG. 26 and the summary survival data are shown in FIG. 27 and Table 21.

TABLE 21
Median Survival Times of Groups Treated with
Increasing Doses of M AAC HPV Median Survival (Days)

| Group | ELN103058 | ELN103212 | ELN133 | ELN1069 |
|---|---|---|---|---|
| PBS | 32.0 | 34.0 | 32.5 | 31.0 |
| IB | 56.0**** | 50.5**** | 57.0*** | 46.0**** |
| M-AAC-HPV | | | | |
| 250M | 49.0*** | 38.0 | 47.0* | 42.0**** |
| M-AAC-HPV | | | | |
| 100M | NA | 34.0 | 39.5 | 39.0*** |
| M-AAC-HPV | | | | |
| 50M | 38.5 | 34.0 | NA | NA |
| M-AAC-HPV | | | | |

NA denotes “not applicable”. Group was not included in study. Statistical significance of a M-AAC-HPV group at a given dose compared to the PBS group shown. *denotes p<0.05, ***denotes p<0.001, ****denotes p<0.0001.

These studies demonstrate that intravenous immunization of mice with M-AAC-HPV therapeutically inhibits tumor growth and prolongs survival in the HPV-16 E6 and E7-expressing TC-1 mouse tumor model. The ability of M-AAC-HPV to inhibit TC-1 tumor growth and prolong survival of tumor-bearing mice is dependent on the dose of M-AAC-HPV. Specifically, mice treated with an intravenously administered M-AAC-HPV dose of 1×10⁹ or 250×10⁶ per mouse exhibited slowed tumor growth compared to mice administered PBS in all (4 of 4) studies using the TC-1 model. Mice administered a dose of 100×10⁶ M-AAC-HPV exhibited slowed tumor growth in comparison to mice administered PBS in the majority (2 of 3) studies. Furthermore, a significant increase in median survival was observed in 4 of 4 studies with the 1×10⁹ dose, in 3 of 4 studies with the 250×10⁶ dose and 1 of 3 studies with the 100×10⁶ dose. Neither the inhibition of tumor growth nor the extension of median survival was observed in mice administered a 50×10⁶ dose (0 of 2 studies).

### Example 14. In Vivo Determination of Efficacy in Mice Following Therapeutic Immunization with Intravenously Administered M-AAC-HPV in the TC-1 Tumor Model—Effect of Boosting Regimen

The objective of this study was to assess the anti-tumor activity of two administrations (a prime and a boost) of intravenously administered M-AAC-HPV at 100×10⁶ or 250×10⁶ M-AAC-HPV per mouse compared to a single administration (prime only) at the same dose in the TC-1 tumor model.

The methods for isolating mouse RBCs and SQZ processing RBCs with E7 SLP and poly I:C to generate the mouse prototype of AAC-HPV (M-AAC-HPV) are described above.

The following tables illustrate the design of the studies to evaluate different doses of M-AAC-HPV (Table 22 and Table 23) in the tumor studies. The mice (female C57BL/6J) were injected subcutaneously (SC) with the TC-1 tumor cells (50,000 cells) on Day 0. Per the schedules described in Table 22 and Table 23, the mice were treated with the test articles by retro-orbital administration.

TABLE 21
Anti-Tumor Effect of M-AAC-HPV with Boosting (ELN133)
ELN133

| | | | M-AAC-HPV | M-AAC-HPV | | | |
|---|---|---|---|---|---|---|---|
| | | | Dose Per | Final Concentration | Total Volume | Study Schedule | |
| Groups | | Mice (#) | Mouse (#/Mouse) | for Injection (#/mL) | Injected (μL) | Day 10 | Day 12 |
| A | PBS | 10 | NA | NA | 200 | NA | NA |
| B | M-AAC-HPV | 10 | 250 × 10⁶ | 1.25 × 10⁹ | 200 | Prime | NA |
| C | M-AAC-HPV | 10 | 250 × 10⁶ | 1.25 × 10⁹ | 200 | Prime | Boost |
| D | M-AAC-HPV | 10 | 100 × 10⁶ | 500 × 10⁶ | 200 | Prime | NA |
| E | M-AAC-HPV | 10 | 100 × 10⁶ | 500 × 10⁶ | 200 | Prime | Boost |

NA denotes “not applicable”

TABLE 22
Anti-Tumor Effect of M-AAC-HPV with Boosting (Study ELN1069)
ELN1069

| | | | M-AAC-HPV | M-AAC-HPV | Total | | |
|---|---|---|---|---|---|---|---|
| | | | Dose Per | Final Concentration | Volume | Study Schedule | |
| Groups | | Mice (#) | Mouse (#/Mouse) | for Injection (#/mL) | Injected (μL) | Day 10 | Day 12 |
| A | PBS | 10 | NA | NA | 100 | NA | NA |
| B | M-AAC-HPV | 10 | 250 × 10⁶ | 2.5 × 10⁹ | 100 | Prime | NA |
| C | M-AAC-HPV | 10 | 250 × 10⁶ | 2.5 × 10⁹ | 100 | Prime | Boost |
| D | M-AAC-HPV | 10 | 100 × 10⁶ | 1 × 10⁹ | 100 | Prime | NA |
| E | M-AAC-HPV | 10 | 100 × 10⁶ | 1 × 10⁹ | 100 | Prime | Boost |

NA denotes “not applicable”

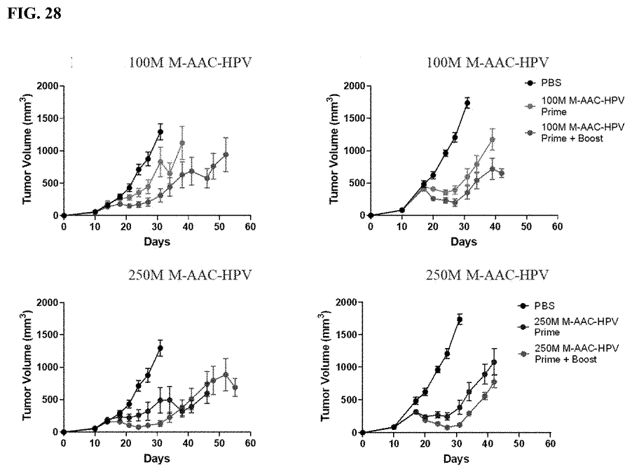

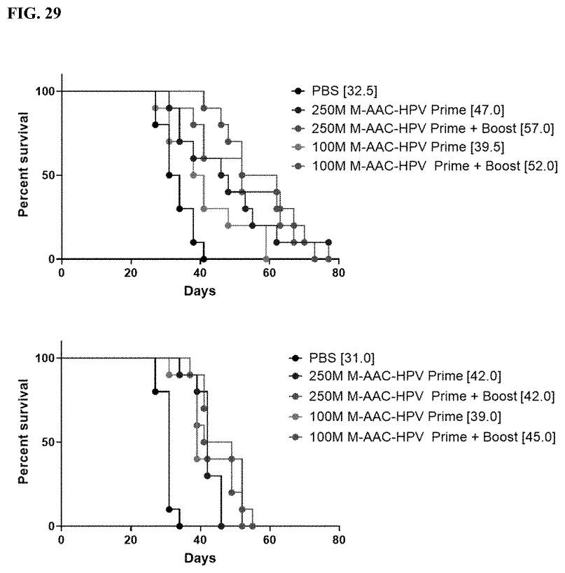

The summary tumor growth data from two independent experiments are shown in FIG. 28. The summary survival data are shown in FIG. 29 and Table 24.

TABLE 24
Median Survival Times of Groups Treated with
Increasing Doses of M-AAC-HPV
Median Survival (Days)

| Group | | ELN133 | ELN1069 |
|---|---|---|---|
| A | PBS | 32.5 | 31.0 |
| B | 250M M-AAC-HPV Prime | 47.0 | 42.0 |
| C | 250M M-AAC-HPV Prime Plus Boost | 57.0 | 42.0 |
| D | 100M M-AAC-HPV Prime | 39.5 | 39.0 |
| E | 100M M-AAC-HPV Prime Plus Boost | 52.0 | 45.0 |

These studies demonstrate that two intravenous administrations of M-AAC-HPV (a prime plus a boost) can lead to slower tumor growth, without an effect on median survival relative to administration of a prime alone in the TC-1 tumor model. Mice treated with a prime plus boost on day 2 when compared to a prime alone of M-AAC-HPV at a dose of 100×10⁶ AACs/mouse showed statistically significant slower tumor growth in 1 out of 2 studies, and a possible trend toward slower growth in the second study. There was no statistical difference at this dose level in the median survival observed in 2 out of 2 studies for prime plus boost as compared to prime alone. Mice treated with a prime plus boost on day 2 of M-AAC-HPV at a dose of 250×10⁶ AACs/mouse showed statistically significant slowed tumor growth when compared to a prime alone in 1 out of 2 studies, and a possible trend toward slower growth in the second study. There was no statistical difference in the median survival data at this dose level in 2 out of 2 studies for prime plus boost as compared to prime alone. Lastly, treatment of mice with a prime or prime plus boost of 100×10⁶ or 250×10⁶ M-AAC-HPV per mouse can delay tumor growth and extend median survival compared to control PBS-treated mice.

### Example 15. In Vivo Immunization of TC-1 Tumor Bearing Mice with M-AAC-HPV to Measure Recruitment of E7-Specific CD8⁺ TILs

The objective of this study was to quantify E7-specific CD8⁺ T cells in the tumor microenvironment of TC-1 tumors 12 days post intravenous immunization with M AAC-HPV.

The methods for isolating mouse RBCs and SQZ processing RBCs with E7 SLP and poly I:C to generate the mouse prototype of AAC-HPV (M-AAC-HPV) are described above.

Table 25 illustrates the design of the studies used to quantify E7-specific CD8⁺ T cells in TC-1 tumors following M-AAC-HPV administration. The mice (female C57BL/6J; 5 per group) were injected subcutaneously with the TC-1 tumor cells (50,000 cells) on day 0. On day 14 (study in ELN68) or 13 (study in ELN221) of the study, the mice were immunized with the test articles as described in Table 25. Tumor volume and survival were monitored until the day before sacrifice (day 24 or day 25). Mice were sacrificed 12 days after test article administration (day 26 in ELN68 and day 25 in ELN221) and tumors were removed for enzymatic processing into single cell suspensions. Tetramer staining was performed on the cell suspensions to determine the percentage of infiltrating CD8+ T cells specific for E7 by flow cytometry.

TABLE 24
Groups to Assess
E7-specific CD8⁺ TILs

| Group | Mice (#) | M-AAC- | Total Volume |
|---|---|---|---|
| | | HPV (#/Mouse) | Injected (uL) |
| A: PBS | 5 | NA | 200 |
| B: M-AAC-HPV | 5 | 250 × 10⁶ | 200 |

NA denotes “not applicable”

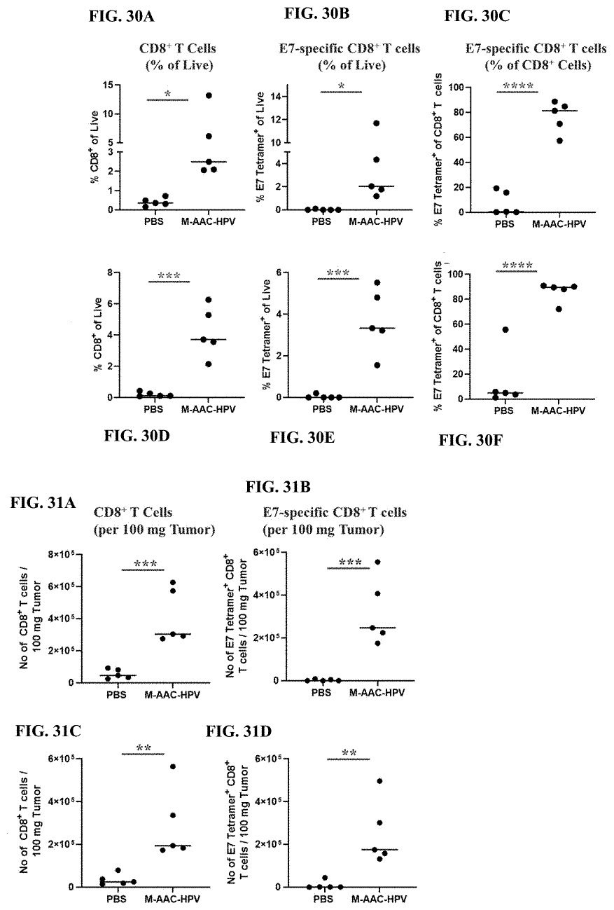

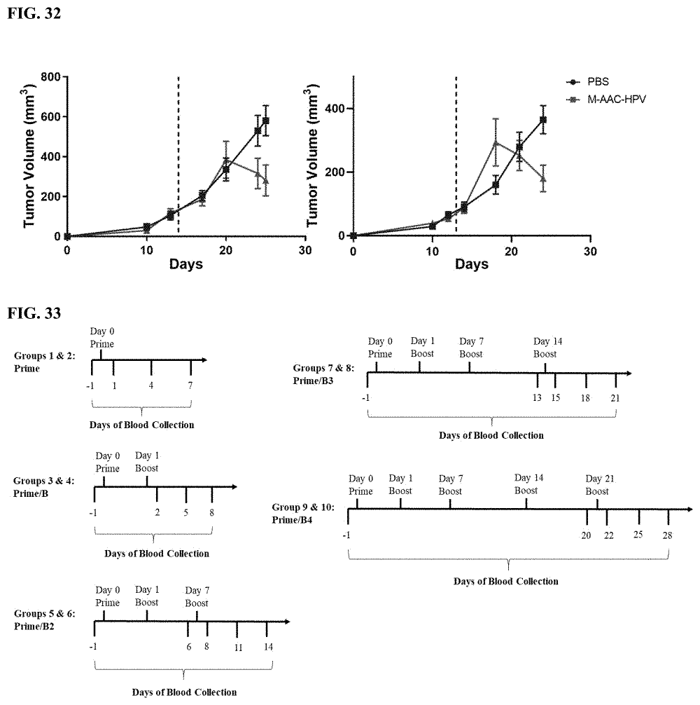

FIG. 30 depicts the intratumoral percentage of CD8⁺ T cells among total live cells, the percentage of E7 tetramer⁺ cells among total live cells, and the percentage of E7 tetramer⁺ cells of the CD8⁺ T cell population. Total CD8⁺ T cells and E7-specific CD8⁺ T cells normalized to tumor mass are shown in FIG. 31. Tumor growth summary data for the mice sacrificed for tumor collection are shown in FIG. 32.

As seen in FIG. 30, mice immunized with M-AAC-HPV had a 12.8-fold and 20.8-fold increase in the mean percentage of CD8⁺ T cells in the tumor compared to PBS-treated controls 12 days after immunization in ELN68 and ELN221, respectively. In M-AAC-HPV-treated animals, the majority of these CD8⁺ T cells were specific for the E7 antigen as determined by tetramer staining (76.6±12.6% in ELN68 and 86.2±7.9% in ELN221 of the CD8⁺ T cell population). These data demonstrate that immunization with M-AAC-HPV significantly increased the mean percentage of E7-specific CD8⁺ T cells in the tumor microenvironment 12 days post immunization in comparison to the PBS treatment (209.3-fold increase in ELN68 and 71.2-fold increase in ELN221). As shown in FIG. 31, this increase in E7 specific CD8⁺ T cells was also noted when converted to cell number normalized to tumor mass. The increase in E7-specific CD8⁺ T cells coupled with the decrease in tumor volume suggests that M-AAC-HPV reduces tumor burden by expanding E7-specific effector CD8⁺ T cells.

These studies demonstrated that intravenous immunization with M-AAC-HPV in the TC-1 mouse tumor model led to a significant increase in E7-specific CD8⁺ T cells infiltrating the tumor. This observation aligns with the proposed mechanism of action for M-AAC-HPV.

### Example 16. In Vivo Serum Cytokine/Chemokine Analysis in Mice Following Repeat Intravenous Administration of M-AAC-HPV, Measured by Luminex Analysis

The objective of this study was to measure serum cytokines/chemokines in mice at various timepoints after intravenous immunization with either 1, 2, 3, 4 or 5 doses of M-AAC-HPV compared to control PBS injected mice.

The methods for isolating mouse RBCs and SQZ processing RBCs with E7 SLP and poly I:C to generate the mouse prototype of AAC-HPV (M-AAC-HPV) are described above.

Table 27 illustrates the design of the study to evaluate serum cytokine/chemokine concentrations in C57BL/6J female mice immunized with up to 5 doses of M-AAC-HPV. Analysis of cytokine/chemokine concentrations using the Milliplex assay was performed following serum collection at all time points as indicated in FIG. 32.

TABLE 27
Study design

| | | | | | M-AAC-HPV | Total Volume | | Serum |
|---|---|---|---|---|---|---|---|---|
| | | | | Dose | Final Concentration | Injected | Immunization | Collection Days |
| Group | | Treatment | Mice (#) | (#/per Mouse) | for Injection (#/mL) | IV (μL) | Days | (Study Days) |
| 1 | Prime | PBS | 10 | 0 | 0 | 100 | 0 | −1^{a}, 1^{a}, 4^{a}, 7^{b} |
| 2 | Prime | M-AAC-HPV | 10 | 0.25 × 10⁹ | 2.5 × 10⁹ | 100 | 0 | −1^{a}, 1^{a}, 4^{a}, 7^{b} |
| 3 | Prime/B | PBS | 10 | 0 | 0 | 100 | 0, 1 | −1^{a}, 2^{a}, 5^{a}, 8^{b} |
| 4 | Prime/B | M-AAC-HPV | 10 | 0.25 × 10⁹ | 2.5 × 10⁹ | 100 | 0, 1 | −1^{a}, 2^{a}, 5^{a}, 8^{b} |
| 5 | Prime/B2 | PBS | 10 | 0 | 0 | 100 | 0, 1, 7 | −1^{a}, 6^{a}, 8^{a}, |
| | | | | | | | | 11^{a}, 14^{b} |
| 6 | Prime/B2 | M-AAC-HPV | 10 | 0.25 × 10⁹ | 2.5 × 10⁹ | 100 | 0, 1, 7 | −1^{a}, 6^{a}, 8^{a}, |
| | | | | | | | | 11^{a}, 14^{b} |
| 7 | Prime/B3 | PBS | 10 | 0 | 0 | 100 | 0, 1, 7, 14 | −1^{a}, 13^{a}, 15^{a}, |
| | | | | | | | | 18^{a}, 21^{b} |
| 8 | Prime/B3 | M-AAC-HPV | 10 | 0.25 × 10⁹ | 2.5 × 10⁹ | 100 | 0, 1, 7, 14 | −1^{a}, 13^{a}, 15^{a}, |
| | | | | | | | | 18^{a}, 21^{b} |
| 9 | Prime/B4 | PBS | 10 | 0 | 0 | 100 | 0, 1, 7, 14, 21 | −1^{a}, 20^{a}, 22^{a}, |
| | | | | | | | | 25^{a}, 28^{b} |
| 10 | Prime/B4 | M-AAC-HPV | 10 | 0.25 × 10⁹ | 2.5 × 10⁹ | 100 | 0, 1, 7, 14, 21 | −1^{a}, 20^{a}, 22^{a}, |
| | | | | | | | | 25^{a}, 28^{b} |

B denotes boost; All intravenous administrations were by the retro-orbital (RO) route.
^{a}Serum was collected via puncture of the submandibular vein.
^{b}Serum was collected by a terminal cardiac puncture.

Analytes included in the Milliplex assay are as follows: G-CSF, GM-CSF, IFN-γ, IL-1α, IL-1β, IL-2, IL-4, IL-5, IL-6, IL-7, IL-9, IL-10, IL-12p40, IL-12p70, IL-13, IL-15, IL-17, IP-10, KC, MCP-1, MIP-1α, MIP-1β, MIP-2, RANTES and TNF-α. Table 28 is a summary table describing key findings for any cytokines/chemokines that exhibited statistical difference in at least one timepoint relative to the corresponding PBS time points.

Four chemokines, namely IP-10, MIP-1β, MCP-1 and RANTES demonstrated a significant, consistent, but transient increase in serum concentrations 1 day after the last immunization in all groups with one exception. The fold change in MIP-1β over pre-immunization values in one of the groups (P/B3) did not significantly differ (p=0.46) between M-AAC-HPV and PBS controls at any time point throughout the course of the study. IP-10 concentrations remained elevated in all groups until 4 days after the immunization. By day 7, the fold change (relative to pre-immunization values) of all cytokines/chemokines in the M-AAC-HPV group were no longer significantly elevated compared to the change observed in PBS controls, with the exception of IL-12p70. The fold change in IL-12p70 remained significantly higher than that observed in the corresponding PBS group on day 7 in 1 group (prime alone group) out of 5 groups that received M-AAC-HPV. However, the changes over the pre-immunization values of IL-12p70 in mice that received M-AAC-HPV in the prime alone group (range 0.43-2.05) were within the range observed in the corresponding PBS group throughout the study (range 0.43-2.37). The concentrations of the other cytokines/chemokines evaluated were not consistently significantly elevated, although, for some other cytokines/chemokines, sporadic statistically significant elevations were observed. These included GM-CSF, IL-7, IL-12p40, IL-12p70, IL-13, KC and MIP-1α.

Adjuvants such as poly I:C have been previously shown to be an activator of the innate immune system leading to the secretion of chemokines including IP-10, MIP-1β, MCP-1 and RANTES by a variety of cell types (Longhi 2015; De Waele 2018). These specific chemokines have also been shown to be important for the migration of CD4+ and CD8⁺ T cells to antigen presenting cell (APC)-rich regions of secondary lymphoid organs, such as the spleen (reviewed in Sokol 2015). Furthermore, they have also been shown to promote clustering and formation of stable contacts between T cells and APCs thereby promoting productive activation and differentiation of naïve T cells into effector T cells. Therefore, the early and transient increase in serum levels of IP-10, MIP-1β, MCP-1 and RANTES possibly indicates early activation of innate immune cells by M-AAC-HPV.

Other analytes that were measured in this study including G-CSF, IFN-γ, IL-1β, IL-1α, IL-2, IL-4, IL-5, IL-6, IL-9, IL-10, IL-15, IL-17, MIP-2 and TNF-α showed no significant change in all M-AAC-HPV groups compared to PBS treated controls at all timepoints.

This study demonstrates that intravenous administration of M-AAC-HPV, generally resulted in a significant but transient elevation in the serum concentrations of IP-10, MIP-1β, MCP-1 and RANTES compared to the respective PBS controls starting at 1 day after the final immunization. Changes in the concentrations of all analytes over pre-immunization values were comparable between mice that received M-AAC-HPV and mice that received PBS by day 7 after the last vaccination.

TABLE 27
Summary TABLE of Cytokines/Chemokines that Show Significant Increase After M-
AAC-HPV Compared to PBS Controls

| | | Time | |
|---|---|---|---|
| | | Point(s) | |
| | | Exhibiting | |
| Cytokine | Group | Differences | Comments |
| GM- | P/B | 1 day after | By day 4 after immunization, the fold change in GM-CSF over pre- |
| CSF | | immunization | immunization values in all mice that received M-AAC-HPV was no longer |
| | | | significantly different than the group that received PBS (p = 0.1). |
| IL-7 | Prime | 4 days | By day 7 after immunization, the fold change in mice that received M-AAC- |
| | | | HPV was no longer significantly different compared to mice that received PBS |
| | | | (p = 0.3). No significant increase in IL-7 was observed in any of the other groups |
| | | | that received M-AAC-HPV. |
| | | after | |
| | alone | immunization | |
| IL- | Prime | 1 and 4 | By day 7 after immunization, the fold change in mice that received M-AAC- |
| 12p40 | alone | days after | HPV was no longer significantly different compared to mice that received PBS. |
| | | immunization | No significant increase in IL-12p40 was observed in any of the other groups |
| | | | that received M-AAC-HPV. |
| IL- | Prime | 4 days and | 2 mice in the M-AAC-HPV group had an increase in IL-12p70 over pre- |
| 12p70 | alone | 7 days | immunization values on day 4 (fold change of 3.0 and 4.8) that was above the |
| | | after | range observed in the PBS group (0.4-2.4). By day 7, all fold change over pre- |
| | | immunization | immunizationvalues in mice that received M-AAC-HPV (range 0.43-2.1) were |
| | | | within the range observed in the corresponding PBS group throughout the study |
| | | | (range 0.4-2.4). No significant increase in IL-12p70 was observed in any of the |
| | | | other groups that received M-AAC-HPV. |
| IL-13 | Prime | 1 and 4 | The range of values in the M-AAC-HPV group for fold change over pre- |
| | alone | days after | immunization values on day 1 (range 1.2-3.5) and day 4 (range 1.0-3.5) were |
| | | immunization | within the range of values for fold change over pre-immunization values |
| | | | observed in the PBS group throughout the course of the study (range 0.4-4.3). |
| | | | Furthermore, by day 7 after immunization, the fold change in IL-13 over pre- |
| | | | immunization values in mice that received M-AAC-HPV was no longer |
| | | | significantly different compared to mice that received PBS (p=0.2). No |
| | | | significant increase in IL-13 relative to PBS controls was observed in any of the |
| | | | other groups that received M-AAC-HPV. |
| IP-10 | Prime | 1 day | Significant differences in fold change in IP-10 were observed consistently on |
| | alone, | before, 1 | days 1 and 4 after immunization in all groups that received M-AAC-HPV |
| | P/B, | and 4 days | compared to PBS controls. In the P/B2 and P/B3 groups, the fold change in IP- |
| | P/B2, | after the | 10 over pre-immunization values was elevated 1 day prior to the last |
| | P/B3, | last | immunization as well. However, by day 7 post immunization, the fold change |
| | P/B4 | immunization | in IP-10 levels over pre-immunization values in all groups that received M- |
| | | | AAC-HPV, was not significantly different than the fold change in levels in the |
| | | | respective PBS groups. |
| KC | Prime | 1 day after | The range of values for fold change in KC over pre-immunization values in |
| | alone | immunization | mice that received M-AAC-HPV on day 1 after immunization (range 1.0-1.9) |
| | | | was within the range of values observed in the corresponding PBS controls over |
| | | | the course of the study (range 0.6-2.4). Furthermore, by day 4 after |
| | | | immunization, the fold change in the M-AAC-HPV group was no longer |
| | | | significantly different than the corresponding PBS group (p = 0.5). |
| Cytokine | Group | Time | Comments |
| | | Point(s) | |
| | | Exhibiting | |
| | | Differences | |
| | P/B3 | 1 day after | The range of values for fold change in KC over pre-immunization values in |
| | | immunization | mice that received 4 doses of M-AAC-HPV 1 day after the last immunization |
| | | | (range 1.1-2.5) was within the range of values observed in the corresponding |
| | | | PBS controls over the course of the study (range 0.6-2.9). Furthermore, by day |
| | | | 4 after the final immunization, the fold change in values over pre-immunization |
| | | | values in the M-AAC-HPV group was no longer significantly different than the |
| | | | fold change observed in the corresponding PBS controls. |
| MCP-1 | Prime | 1 day after | A significant increase in MCP-1 was observed in all groups that received M- |
| | alone, | immunization | AAC-HPV compared to the corresponding PBS groups, 1 day after the last |
| | P/B, | | immunization. However, by day 4 after the last immunization, fold change over |
| | P/B2, | | pre-immunization values in all groups that received M-AAC-HPV were no |
| | P/B3, | | longer significant compared to the fold change values in the corresponding PBS |
| | P/B4 | | groups. |
| MIP-lα | P/B | 1 day after | The fold change in MIP-1α over pre-immunization values in mice that received |
| | | immunization | M-AAC-HPV (range 0.3-2.8) was within the range of fold change observed in |
| | | | the PBS group throughout the course of the study (range 0.3-3.3). Furthermore, |
| | | | by day 4 after the last immunization, the fold change in the M-AAC-HPV group |
| | | | was no longer significantly different from the fold change in the corresponding |
| | | | PBS group. |
| MIP-1β | Prime | 1 day after | A significant increase in MIP-1β was observed 1 day after the last |
| | alone, | immunization | immunization in 4 out of the 5 groups that received M-AAC-HPV compared to |
| | P/B, | | the corresponding PBS groups. However, by day 4 after the last immunization, |
| | P/B2, | | fold change over pre-immunization values in all groups that received M-AAC- |
| | P/B4 | | HPV was no longer significant compared to the fold change in the |
| | | | corresponding PBS groups. |
| RANTE | Prime | 1 day after | A significant increase in RANTES was observed 1 day after the last |
| S | alone, | immunization | immunization in all groups that received M-AAC-HPV compared to the |
| | P/B, | | corresponding PBS groups. However, by day 4 after the last immunization, fold |
| | P/B2, | | change over pre-immunization values in all groups that received M-AAC-HPV |
| | P/B3, | | was no longer significantly different compared to the fold change in the |
| | P/B4 | | corresponding PBS groups. |

### Example 17. In Vivo Determination of RBC Clearance in Mice Following Repeat IV Administration of M-AAC-HPV

The objective of this study was to determine if five repeated administrations of M-AAC-HPV result in an immune response against components of the RBCs thereby resulting in accelerated clearance of a subsequent administration of unprocessed RBCs. This study was conducted because previous studies in mice have shown that adjuvants such as polyinosinic-polycytidylic acid (poly I:C) intravenously administered at the time of intravenous blood administration can induce or enhance immune responses against surface RBC antigens (Gibb 2017). Such responses have been shown to result in the accelerated clearance of RBCs in a subsequent intravenous administration (Stowell SR 2014).

The methods for isolating mouse RBCs and SQZ processing RBCs with E7 SLP and poly I:C to generate the mouse prototype of AAC-HPV (M-AAC-HPV) are described above.

Table 29 illustrates the design of the study to evaluate the circulation kinetics of intravenously administered unprocessed (not SQZ processed) syngeneic RBCs (labeled with PKH26) in female C57BL/6J mice immunized with 5 doses of M-AAC-HPV. Analysis of circulating RBCs was performed immediately following blood collection at the time points indicated in Table 29 by flow cytometry for PKH26 labeled RBCs.

TABLE 29
Analysis of circulating RBCs

| | | | | | | Intravenous Administration Study Schedule | | | | |
|---|---|---|---|---|---|---|---|---|---|---|
| | | Immunization Study Schedule | | | | | PKH26 | | | |
| | | | M-AAC-HPV | | | | Labeled | | | Bleed Time |
| | | | Final | Total | | PKH26 | RBC Final | Total | | Points |
| | | | Concentration | Volume | Intravenous | Labeled | Concentration | Volume | Intravenous | (Hours After |
| | Mice | Dose (# Per | for Injection | Injected | Immunization | RBC Dose | for Injection | Injected | Administration | Intravenous |
| Groups | (#) | Mouse) | (#/mL) | (μL) | Days | (#/Mouse) | (#/mL) | (μL) | Day | Administration) |
| A: PBS | 10 | 0 | 0 | 100 | 0, 1, 7, 14, 21 | 1 × 10⁹ | 5 × 10⁹ | 200 | 28 | 0, 2, 4, 24, 48 |
| B: M-AAC- | 10 | 0.25 × 10⁹ | 2.5 × 10⁹ | 100 | 0, 1, 7, 14, 21 | 1 × 10⁹ | 5 × 10⁹ | 200 | 28 | 0, 2, 4, 24, 48 |
| HPV | | | | | | | | | | |

All intravenous administrations were by the retro-orbital (RO) route.
M-AAC-HPV = Mouse Prototype of AAC-HPV (Mouse RBCs SQZ Processed with Mouse E7 SLP and Poly I:C);
PBS = phosphate buffered saline

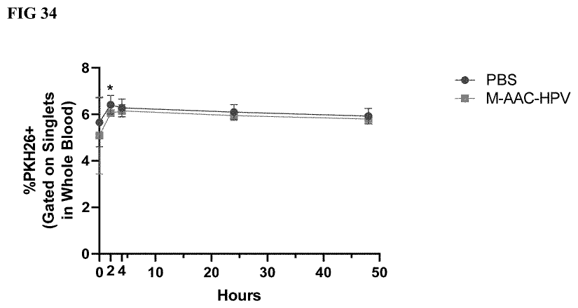

The percentage of labeled RBCs in whole blood at various time points post intravenous administration is shown in FIG. 34.

This study evaluated the impact of repeat dosing of M-AAC-HPV on the induction of immune responses against syngeneic RBCs by measuring the clearance of intravenously administered labeled RBCs. Labeled RBCs were administered one week after the fifth and final immunization of M-AAC-HPV to allow sufficient time for the development of an immune response. Following intravenous administration, labeled RBCs were monitored for a period of 48 hours. There was no statistical difference between the two groups at all timepoints except for the two-hour time point. At the two-hour time point, the value for the percentage of PKH26 labeled RBCs in whole blood for M-AAC-HPV was 6.05% (range: 5.86-6.21%) compared to 6.41% (range: 5.87-7.11%) for PBS, a difference which is within the expected animal to animal variability and is not physiologically significant.

This study demonstrates that intravenous administration of 5 doses of M-AAC-HPV, when compared to 5 doses of PBS control, does not result in immune responses that can cause accelerated clearance of syngeneic RBCs as evidenced by equivalent circulation of intravenously administered syngeneic, labeled RBCs for up to 48 hours after administration.

SEQUENCES

| SEQ ID | | |
|---|---|---|
| NO | Sequence | Description |
| 1 | TIHDIILECV | HPV16-E6(29-38), human |
| | | epitope |
| 2 | EVYDFAFRDL | HPV16-E6(48-57), murine |
| | | epitope |
| 3 | YMLDLQPETT | HPV16-E7(11-20), human |
| | | epitope |
| 4 | RAHYNIVTF | HPV16-E7(49-57), murine |
| | | epitope |
| 5 | LPQLSTELQT | HPV16-E6(19-28) N-terminal |
| | | polypeptide, human |
| 6 | QLCTELQT | HPV16-E6(21-28) N-terminal |
| | | polypeptide, human |
| 7 | KQQLLRR | HPV16-E6(41-47) N-terminal |
| | | polypeptide, native murine |
| 8 | VYSKQQLLRR | HPV16-E6(38-47) N-terminal |
| | | polypeptide, classic murine |
| 9 | MHGDTPTLHE | HPV16-E7(1-10) N-terminal |
| | | polypeptide, human |
| 10 | GQAEPD | HPV16-E7(43-48) N-terminal |
| | | polypeptide, murine |
| 11 | YSKQQLLRREVYDFAF | HPV16-E6(39-54) C-terminal |
| | | polypeptide, human |
| 12 | YCKQQLL | HPV16-E6(39-45) C-terminal |
| | | polypeptide, human |
| 13 | CIVYRDGN | HPV16-E6(58-65) C-terminal |
| | | polypeptide, native murine |
| 14 | SIVYRDGNPYAVSDK | HPV16-E6(58-72) C-terminal |
| | | polypeptide, classic murine |
| 15 | DLYCYEQLNDSSEEE | HPV16-E7(21-35) C-terminal |
| | | polypeptide, human |
| 16 | CCKCDSTLRLCVQSTHVDIR | HPV16-E7(58-77 C-terminal |
| | | polypeptide, native murine |
| 17 | SSKSDSTLRLSVQSTHVDIR | HPV16-E7(58-77) C-terminal |
| | | polypeptide, classic murine |
| 18 | LPQLSTELQTTIHDIILECVYSKQQLLRREVYDFAF | HPV16-E6(19-54) SLP, |
| | | human |
| 19 | QLCTELQTTIHDIILECVYCKQQLL | HPV16-E6(21-45) SLP, |
| | | human |
| 20 | KQQLLRREVYDFAFRDLCIVYRDGN | HPV16-E6(41-65) SLP, native |
| | | murine |
| 21 | VYSKQQLLRREVYDFAFRDLSIVYRDGNPYAVSDK | HPV16-E6(38-72) SLP, classic |
| | | murine |
| 22 | MHGDTPTLHEYMLDLQPETTDLYCYEQLNDSSEEE | HPV16-E7(1-35) SLP, human |
| 23 | QLCTELQTYMLDLQPETTYCKQQLL | HPV16-E7.6 SLP, human |
| 24 | GQAEPDRAHYNIVTFCCKCDSTLRLCVQSTHVDIR | HPV16-E7(43-77) SLP, native |
| | | murine |
| 25 | GQAEPDRAHYNIVTFSSKSDSTLRLSVQSTHVDIR | HPV16-E7(43-77) SLP, classic |
| | | murine |
| 26 | ggGGTCAACGTTGAgggggg | ODN 1585 (Class A, mouse- |
| | | Bases shown in capital letters are specific) |
| | | phosphodiester, and those in lower case are |
| | | phosphorothioate |
| 27 | ggGGGACGA:TCGTCgggggg | ODN 2216 (Class A, human- |
| | | Bases shown in capital letters are selective) |
| | | phosphodiester, and those in lower case are |
| | | phosphorothioate |
| 28 | gggGACGAC:GTCGTGgggggg | ODN 2336 (Class A, human |
| | | Bases shown in capital letters are preferred) |
| | | phosphodiester, and those in lower case are |
| | | phosphorothioate |
| 29 | tccatgacgttcctgatgct | ODN 1668 (Class B, mouse |
| | | Bases shown in capital letters are specific) |
| | | phosphodiester, and those in lower case are |
| | | phosphorothioate |
| 30 | tccatgacgttcctgacgtt | ODN 1826 (Class B, mouse |
| | | Bases are phosphorothioate specific) |
| 31 | tcgtcgttttgtcgttttgtcgtt | ODN 2006 (Class B, human |
| | | Bases are phosphorothioate selective) |
| 32 | tcg tcg ttg tcg ttt tgt cgt t | ODN 2007 (Class B, |
| | | Bases are phosphorothioate bovine/porcine) |
| 33 | tcg acg ttc gtc gtt cgt cgt tc | ODN BW006 (Class B, |
| | | Bases are phosphorothioate human & mouse) |
| 34 | tcg cga cgt tcg ccc gac gtt cgg ta | ODN D-SL01 (Class B, |
| | | Bases are phosphorothioate multispecies) |
| 35 | tcgtcgttttcggcgc:gcgccg | ODN 2395 (Class C, |
| | | Bases are phosphorothioate human/mouse) |
| 36 | tcgtcgtcgttc:gaacgacgttgat | ODN M362 (Class C, |
| | | Bases are phosphorothioate human/mouse) |
| 37 | tcg cga acg ttc gcc gcg ttc gaa cgc gg | ODN D-SL03 (Class C, |
| | | Bases are phosphorothioate multispecies) |
| 38 | MHGDTPTLHEYMLDLQPETTDLYCYEQLNDSSEEE | E7 |
| 39 | LYCYEQLNDSSEEEDEIDGPAGQAEPDRAHYNIVT | E7 |
| 40 | GQAEPDRAHYNIVTFCCKCDSTLRLCVQSTHVDIR | E7 |
| 41 | TLRLCVQSTHVDIRTLEDLLMGTLGIVCPICSQKP | E7 |
| 42 | MHQKRTAMFQDPQERPRKLPQLCTELQTTIHD | E6 |
| 43 | LPQLCTELQTTIHDIILECVYCKQQLLRREVY | E6 |
| 44 | KQQLLRREVYDFAFRDLCIVYRDGN | E6 |
| 45 | RDLCIVYRDGNPYAVCDKCLKFYSKI | E6 |
| 46 | DKCLKFYSKISEYRHYCYSLYGTTL | E6 |
| 47 | HYCYSLYGTTLEQQYNKPLCDLLIR | E6 |
| 48 | YGTTLEQQYNKPLCDLLIRCINCQKPLCPEEK | E6 |
| 49 | RCINCQKPLCPEEKQRHLDKKQRFHNIRGRWT | E6 |
| 50 | DKKQRFHNIRGRWTGRCMSCCRSSRTRRETQL | E6 |

1. A method for treating a human papilloma virus (HPV)-associated cancer in an individual in need thereof, the method comprising administering a composition comprising activating antigen carriers (AACs) to the individual at a dose of about 0.1×10⁸ AACs/kg to about 1×10⁹ AACs/kg, wherein the AACs comprise at least one HPV antigen and an adjuvant delivered intracellularly.

2. A method for treating a human papilloma virus (HPV)-associated cancer in an individual in need thereof, the method comprising administering to the individual:
(i) a composition comprising activating antigen carriers (AACs) and (ii) an immune checkpoint inhibitor which is selected from an antagonist of CTLA-4 (CTLA-4 antagonist), antagonist of PD-1 (PD-1 antagonist), antagonist of PD-L1 (PD-L1 antagonist), or a combination thereof, wherein the AACs comprise at least one HPV antigen and an adjuvant delivered intracellularly.

3-10. (canceled)

11. The method of claim 1, wherein the at least one HPV antigen comprises a HPV-16 antigen, a HPV-18 antigen, or both.

12. The method of claim 11, wherein the at least one HPV antigen comprises a peptide derived from HPV E6, E7, or both.

13-14. (canceled)

15. The method of claim 1, wherein the at least one HPV antigen comprises the amino acid sequence set forth in any one of SEQ ID NOs:1-4 and 18-25.

16. The method of claim 1, wherein the AACs comprise an antigen comprising the amino acid sequence of SEQ ID NO: 19 and an antigen comprising the amino acid sequence of SEQ ID NO: 23.

17. The method of claim 1, wherein the adjuvant comprises a CpG oligodeoxynucleotide (ODN), a LPS, an IFN-α, a STING agonist, a RIG-I agonist, a poly I:C, R837, R848, a TLR3 agonist, a TLR4 agonist, a TLR 9 agonist, or a combination thereof.

18-22. (canceled)

23. The method of claim 1, wherein the HPV-associated cancer comprises a head and neck cancer a cervical cancer, an anal cancer, an esophageal cancer, or a combination thereof.

24-26. (canceled)

27. The method of claim 1, wherein the composition is administered to the individual at a dose of about 0.5×10⁸ AACs/kg to about 1×10⁹ AACs/kg.

28. The method of claim 1, wherein the composition is administered to the individual at a dose of about 0.5×10⁸ AACs/kg to about 7.5×10⁸ AACs/kg.

29. (canceled)

30. The method of claim 1, wherein the CTLA-4 antagonist is administered to the individual at a dose of about 1 mg/kg to about 3 mg/kg, the PD-1 antagonist is administered to the individual at a dose of about 360 mg, the PD-L1 antagonist is administered to the individual at a dose of about 1200 mg, or a combination thereof.

31-32. (canceled)

33. The method of claim 1, wherein the composition is administered to the individual on day 1 of a three-week cycle.

34. The method of claim 1, wherein the composition is further administered to the individual on day 2 of a first three-week cycle.

35-38. (canceled)

39. The method of claim 1, wherein the immune checkpoint inhibitor is administered to the individual once per three-week cycle or once per two three-week cycles.

40. (canceled)

41. The method of claim 39, wherein the CTLA-4 antagonist is administered on day 1 of each three-week cycle or day 1 of two three-week cycles.

42. (canceled)

43. The method of claim 39, wherein the PD-1 antagonist is administered on day 8 of the first three-week cycle and day 1 of each subsequent cycle.

44-45. (canceled)

46. The method of claim 39, wherein the PD-L1 antagonist is administered on day 8 of the first three-week cycle and day 1 of each subsequent cycle.

47-51. (canceled)

52. The method of claim 1, wherein the at least one HPV antigen and the adjuvant are delivered intracellularly to the AACs by
passing a cell suspension comprising a population of input anucleate cells through a cell-deforming constriction, thereby causing perturbations of the input anucleate cells, and wherein the at least one HPV antigen and the adjuvant to enter the input anucleate cells through the perturbations when contacted with the input anucleate cells to generate the AACs comprising the at least one HPV antigen and the adjuvant.

53. The method of claim 52, wherein the diameter of the constriction is about 1.6 μm to about 2.4 μm or about 1.8 μm to about 2.2 μm.

54. The method of claim 52, wherein the input anucleate cell comprises a red blood cell.

55-60. (canceled)
